(19) United States
(12) Patent Application Publication
(10) Pub. No.: US 2010/0056508 A1
(43) Pub. Date: Mar. 4, 2010

(54) AMINE DERIVATIVES AND THEIR USE IN BETA-2-ADRENORECEPTOR MEDIATED DISEASES

(75) Inventors: Lilian Alcaraz, Leicestershire (GB); Roger Victor Bonnert, Leicestershire (GB); Stephen Connolly, Leicestershire (GB); Anthony Ronald Cook, Leicestershire (GB); Adrian Fisher, Leicestershire (GB); Alexander Humphries, Leicestershire (GB); Piotr Raubo, Leicestershire (GB)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/519,591

(22) PCT Filed: Dec. 19, 2007
(86) PCT No.: PCT/GB07/04859

§ 371 Date: Jun. 17, 2009

Related U.S. Application Data
(60) Provisional application No. 60/870,956, filed on Dec. 20, 2006.

(51) Int. Cl. A61K 31/536; C07D 265/36; A61K 31/16; C07C 237/00

(52) U.S. Cl. 514/230.5; 544/105; 514/626; 564/193

(57) ABSTRACT

The present invention provides compounds of formula (I), wherein k, Ar, R², R³, R⁴, R⁵, R^{4′}, R^{5′}, R⁶, R⁷, A, D, m and E are as defined in the specification, processes for their preparation, pharmaceutical compositions containing them and their use in therapy.

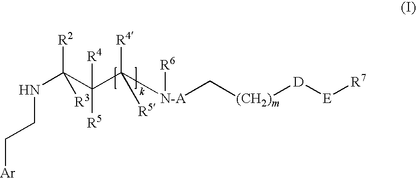

The present invention relates to amine derivatives, processes for their preparation, pharmaceutical compositions containing them and their use in therapy (for example their use in β2 adrenoreceptor mediated disease states).

Adreneoceptors are a group of G-protein coupled receptors divided into two major sub-families, α and β. These sub-families are further divided into sub-types of which the β sub-family has at least 3 members: β1, β2 and β3. β2 adrenoceptors (henceforth referred to as β2 receptors) are mainly expressed on smooth muscle cells.

Agonism of the β2 receptor on airway smooth muscle produces relaxation and therefore bronchodilatation. Through this mechanism, β2 agonists act as functional antagonists to all bronchoconstrictor substances such as the naturally-occurring histamine and acetylcholine as well as the experimental substances methacholine and carbachol. β2 agonists are widely used to treat airways diseases including asthma and chronic obstructive pulmonary disease (COPD), and this has been extensively reviewed in the literature and incorporated into national guidelines for the treatment of these diseases (British Guideline on the Management of Asthma, NICE guideline No. 12 on the Management of COPD).

β2 agonists are classed either as short-acting or long-acting. Short-acting β2 agonists (SABAs) such as salbutamol have a duration of action of 2-4 hours. They are suitable for rescue medication during a period of acute bronchoconstriction but are not suitable for continuous medication because the beneficial effect of these drugs wears off during the night. Long-acting β2 agonists (LABAs) currently have a duration of action of about 12 hours and are administered twice daily to provide continuous bronchodilatation. They are particularly effective when administered in combination with inhaled corticosteroids. This benefit is not seen when inhaled corticosteroids are combined with SABAs (Kips and Pauwels, Am. J. Respir. Crit. Care Med., 2001, 164, 923-932). LABAs are recommended as add-on therapy to patients already receiving inhaled corticosteroids for asthma to reduce nocturnal awakening and reduce the incidence of exacerbations of the disease. Corticosteroids and LABAs are conveniently co-administered in a single inhaler to improve patient compliance.

There are shortcomings to existing LABAs and there is a need for a new drug in this class. Salmeterol, a commonly used LABA, has a narrow safety margin and side effects related to systemic agonism of β2 receptors (such as tremor, hypokalaemia, tachycardia and hypertension) are common. Salmeterol also has a long onset of action which precludes its use as both a rescue and a maintenance therapy. All current LABAs are administered twice daily and there is a medical need for once daily treatments to improve treatment and patient compliance. Such once daily compounds, co-administered with corticosteroids, will become the mainstay of asthma treatment (Barnes, Nature Reviews, 2004, 3, 831-844). The advantages of once-daily bronchodilator treatment in COPD has been demonstrated with tiotropium, a non-selective muscarinic antagonist (Koumis and Samuel, Clin. Ther. 2005, 27(4), 377-92). There is, however, a need for a once-daily LABA for the treatment of COPD to avoid the side effects of anti-muscarinics such as tiotropium.

Benzothiazolone derivatives having dual β2 receptor and dopamine (D2) receptor agonist properties are known from WO 92/08708, WO 93/23385 and WO 97/10227.

The present invention provides a compound of formula (I):

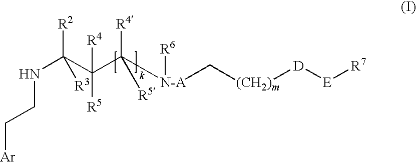

wherein:

### Ar is

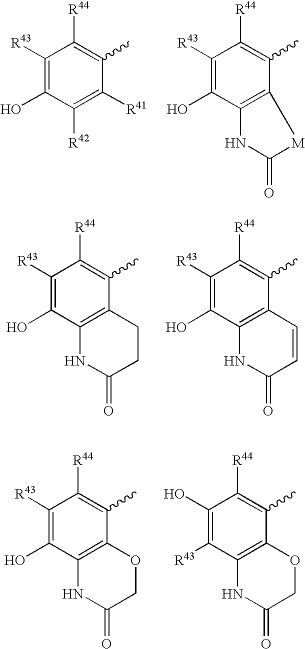 [0009] each of R², R³, R⁴, R⁵, R^{4′} and R^{5′} is, independently, hydrogen or C₁₋₆ alkyl;
A is CH₂, C(O) or S(O)₂;
D is oxygen, sulphur or NR⁸
E is a bond, CR⁶³R⁶⁴, CR⁶³R⁶⁴CR⁶⁵R⁶⁶, CR⁶³R⁶⁴CR⁶⁵R⁶⁶CR⁶⁷R⁶⁸ or CR⁶³R⁶⁴CR⁶⁵R⁶⁶CR⁶⁷R⁶⁸CR⁶⁹R⁷⁰;
R⁶³ and R⁶⁴ being, independently, hydrogen, C₁₋₄ alkyl; and when D is oxygen R⁶³ and
R⁶⁴ can also be fluoro;
R⁶⁵, R⁶⁶, R⁶⁷, R⁶⁸, R⁶⁹ and R⁷⁰ are, independently, hydrogen, fluoro or C₁₋₄ alkyl;
k is 0 or 1;
m is an integer from 0 to 3;
R⁶ is a group —(X)ₚ—Y—(Z)_{q}—R¹⁰, or R⁶ is an α- or β-branched C₃₋₁₂ alkyl (optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁵⁸R⁵⁹, OC(O)(C₁₋₆ alkyl), C₃₋₁₂ cycloalkyl or R⁶⁰);
X and Z are, independently, a C₁₋₆ allylene group optionally substituted with halogen, trifluoromethyl, amino (NH₂), (di)-C₁₋₆ alkylamino, (di)-C₁₋₆ alkylaminocarbonyl, C₁₋₆ alkylcarbonylamino, sulphonamido (—SO₂NH₂) or (di)-C₁₋₆ alkylaminosulphonyl;
p and q are, independently, 0 or 1;
Y is a bond, oxygen, sulphur, CH₂, C(O) or NR⁹; provided that when p is 0 Y is not sulphur;
M is C(O), NR⁵¹ or CR⁵²R⁵³; but when R⁶ is C₁₋₆ alkyl then M is not CH₂ or NR⁵¹;
R⁸ is hydrogen or C₁₋₆ alkyl;
R⁹ is hydrogen or C₁₋₆ alkyl;
R¹⁰ is hydrogen, or a saturated or unsaturated 3- to 12-membered ring system optionally comprising a ring heteroatom selected from nitrogen, oxygen and sulphur, the ring system being optionally substituted by halogen, trifluoromethyl, cyano, carboxyl, hydroxy, —S(O)ᵣR¹⁵, —NR¹⁶S(O)ₛR¹⁷, —C(O)NR¹⁸R¹⁹, —NHC(O)R²⁰, C₁₋₆ alkyl, C₁₋₆ alkoxy, C₁₋₆ alkylcarbonyl, C₁₋₆ alkoxycarbonyl or a saturated or unsaturated 4- to 7-membered monocyclic ring system optionally comprising at least one ring heteroatom selected from nitrogen, oxygen and sulphur, the monocyclic ring system itself being optionally substituted by halogen, trifluoromethyl, hydroxy, —NR²¹S(O)ₜR²², —NHC(O)R²³ or C₁₋₆ alkoxy;
R⁷ is a 5- to 14-membered aromatic or heteroaromatic ring system optionally substituted by halogen, trifluoromethyl, hydroxy, carboxyl, C₁₋₆ alkyl (optionally substituted by halogen or —NR²⁴R²⁵), C₁₋₆ alkoxy (optionally substituted by halogen or —NR²⁶R²⁷), C₃₋₆ cycloalkyl, C₂₋₄ alkenyl, C₂₋₄ alkynyl, cyano, C₁₋₆ alkoxycarbonyl, —NR²⁸R²⁹, C₁₋₆ alkylcarbonylamino, OC(O)(C₁₋₆ alkyl), C₁₋₆ alkylthio, C₁₋₆ alkylS(O), C₁₋₆ alkylS(O)₂, C₁₋₆ alkylsulphonylamino, phenylsulphonylamino, —C(O)NHR³⁰, —SO₂NHR³³, C₀₋₆ alkyl-R³⁴, or a phenyl or 5- to 6-membered heteroaromatic ring (each of which is optionally substituted by halogen, trifluoromethyl, hydroxy, C₁₋₆ alkyl, C₁₋₆ alkoxy or —NR³⁵R³⁶);
R²⁴, R²⁵, R²⁶, R²⁷, R²⁸, R²⁹, R⁵⁸ and R⁵⁹ are, independently, hydrogen or C₁₋₆ alkyl;
R³⁰ is hydrogen, C₁₋₆ alkyl, phenyl-C₀₋₆ alkyl or C₂₋₆ alkylene-NR³¹R³²;
R³¹ and R³² are, independently, hydrogen or C₁-C₆ alkyl, or R³¹ and R³² together with the nitrogen atom to which they are attached form a 4- to 6-membered saturated heterocyclic ring optionally comprising a further ring heteroatom selected from nitrogen and oxygen;
R³³ is hydrogen, C₁₋₆ alkyl, phenyl-C₀₋₆ alkyl or C₂₋₆ allylene-NR³⁷R³⁸;
R³⁴ is a saturated, 5- or 6-membered nitrogen-containing ring;
R³⁵ and R³⁶ are, independently, hydrogen or C₁₋₆ alkyl;
R³⁷ and R³⁸ are, independently, hydrogen or C₁₋₆ alkyl, or R³⁷ and R³⁸ together with the nitrogen atom to which they are attached form a 4- to 6-membered saturated heterocyclic ring optionally comprising a further ring heteroatom selected from nitrogen and oxygen;
   R⁶⁰ is a 4- to 7-membered heterocyclyl comprising a ring nitrogen (optionally substituted by C(O)(C₁₋₆ alkyl)), oxygen or sulphur; the ring R⁶⁰ being optionally substituted by C₁₋₆ alkyl, and ring carbon atoms which are not adjacent to a ring heteroatom are optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁶¹R⁶², OC(O)(C₁₋₆ alkyl or C₃₋₁₂ cycloalkyl;
   R⁴¹, R⁴², R⁴³ and R⁴⁴ are, independently, hydrogen, halogen, trifluoromethyl, cyano, carboxyl, hydroxy, —S(O)_{V}R⁴⁵, —NR⁴⁶S(O)_{W}R⁴⁷, —C(O)NR⁴⁸R⁴⁹, —NR⁵⁵C(O)R⁵⁰, C₁₋₆ alkyl, C₁₋₆ alkoxy, C₁₋₆ alkylcarbonyl or C₁₋₆ alkoxycarbonyl;
   R⁴² can also be CH₂OH, NHC(O)H, NHS(O)₂R⁵⁴ or NHS(O)₂NR⁵⁶R⁵⁷;
   R¹⁵, R¹⁶, R¹⁷, R¹⁸, R¹⁹, R²⁰, R²¹, R²², R²³, R⁴⁵, R⁴⁶, R⁴⁷, R⁴⁸, R⁴⁹, R⁵⁰, R⁵¹, R⁵², R⁵³, R⁵⁴R⁵⁵, R⁵⁶, R⁵⁷, R⁶¹ and R⁶² are, independently C₁₋₆ alkyl; and R¹⁶, R¹⁸, R¹⁹, R²⁰, R²¹, R²³, R⁴⁶, R⁴⁸, R⁴⁹, R⁵⁰, R⁵¹, R⁵², R⁵³, R⁵⁴, R⁵⁵, R⁵⁶, R⁵⁷, R⁶¹ and R⁶² can also be hydrogen; and,
   r and v can be, independently, 0, 1 or 2;
   s, t and w are all 2;
   or a pharmaceutically acceptable salt thereof.

In the context of the present specification, unless otherwise stated, an alkyl substituent group or an alkyl moiety in a substituent group may be linear or branched. Examples of C₁₋₆ alkyl groups/moieties include methyl, ethyl, n-propyl, isopropyl, n-butyl, iso-butyl, tert-butyl, n-pentyl and n-hexyl. Similarly, an alkylene group may be linear or branched. Examples of C₁₋₆ alkylene groups include methylene, ethylene, n-propylene, n-butylene, n-pentylene, n-hexylene, 1-methylethylene, 2-methylethylene, 1,2-dimethylethylene, 1-ethylethylene, 2-ethylethylene, 1-, 2- or 3-methylpropylene and 1-, 2- or 3-ethylpropylene. The allyl moieties in a di-C₁₋₆ alkylamino, di-C₁₋₆ alkylaminocarbonyl or di-C₁₋₆ alkylaminosulphonyl substituent group may be the same or different. In the definition of R¹⁰, the saturated or unsaturated 3- to 12-membered ring system and the saturated or unsaturated 4- to 7-membered monocyclic ring system may each have alicyclic or aromatic properties. An unsaturated ring system will be partially or fully unsaturated. When R³¹ and R³² (or R³⁷ and R³⁸) together represent a 4- to 6-membered saturated heterocyclic ring, it should be understood that the ring will contain no more than two ring heteroatoms: the nitrogen ring atom to which R³¹ and R³² (or R³⁷ and R³⁸) are attached and optionally a nitrogen or oxygen ring atom.

Cycloalkyl is a non-aromatic ring that can comprise one, two or three non-aromatic rings, and is, optionally, fused to a benzene ring (for example to form an indanyl, or 1,2,3,4-tetrahydronaphthyl ring). Examples of cycloalkyl include cyclopropyl, cyclobutyl, cyclopentyl, cyclohexyl, cycloheptyl, bicyclo[2.2.1]heptyl, cyclopentenyl, cyclohexenyl or adamantyl.

A 4- to 7-membered heterocyclyl of R⁶⁰ comprises a ring nitrogen, oxygen or sulphur is, for example, piperidinyl, piperazinyl, pyrrolidinyl, tetrahydropyranyl, tetrahydrothiopyranyl, oxetanyl, tetrahydrofuryl, thietanyl or tetrahydrothienyl.

The compounds of the invention are β2 receptor agonists and possess properties that make them more suitable for once-a-day administration (for example as evidenced by the compounds' long half-life in a memmalian system). In particular, certain compounds of the invention are at least 10-fold more potent at the β2 receptor compared to the α1, β1 or dopamine (D2) receptors. The compounds are also notable for having a fast onset of action that is the time interval between administration of a compound of the invention to a patient and the compound providing symptomatic relief. Onset can be predicted in vitro using isolated trachea from guinea pig.

Incorporation of an α- or β-branched alkyl group as R⁶ advantageously provides increased chemical stability relative to compounds having a straight chain alkyl at this position.

In one particular aspect the present invention provides a compound of formula (I) wherein each of R², R³, R⁴, R⁵ and, if present, R^{4′} and R^{5′} independently represents hydrogen or C₁₋₆, or C₁₋₄, or C₁₋₂ alkyl.

In another embodiment, each of R², R³, R⁴, R⁵ and, if present, R^{4′} and R^{5′} represents hydrogen.

In a further embodiment the present invention provides a compound of formula (I) wherein Ar is:

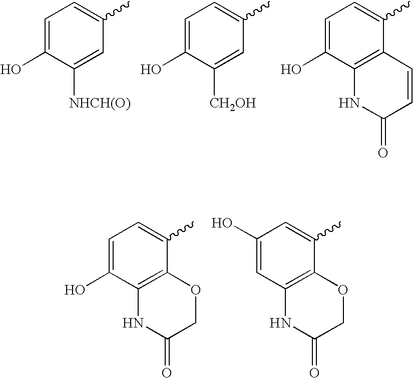

In a still further embodiment the present invention provides a compound of formula (I) wherein Ar is:

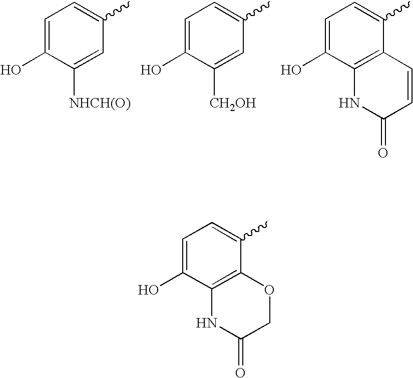

In another embodiment Ar can be:

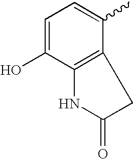

In a still further embodiment the present invention provides a compound of formula (I) wherein Ar is:

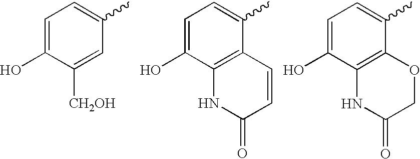

In another embodiment the present invention provides a compound of formula (I) wherein Ar is:

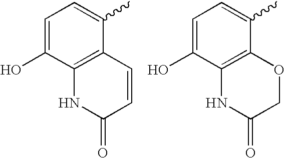

In yet another embodiment Ar is:

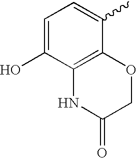

In a further embodiment the present invention provides a compound of formula (I) wherein Ar is:

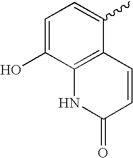

In an embodiment of the invention, A represents C(O).

In another embodiment of the invention, A represents CH₂.

In an embodiment of the invention, D represents oxygen.

In another embodiment of the invention D is NH or N(C₁₋₄ alkyl) (for example NCH₃). For example D is NH.

In a further embodiment of the invention, m is an integer 0, 1, 2 or 3, for example, 1.

In another embodiment of the invention R⁶ is an α- or β-branched C₃₋₁₂ alkyl (optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁵⁸R⁵⁹, OC(O)(C₁₋₆ alkyl), C₃₋₁₂ cycloalkyl or R⁶⁰), wherein R⁵⁸, R⁵⁹ and R⁶⁰ are as defined above.

In yet another embodiment R⁶ is an α- or β-branched C₃₋₁₂ allyl (such as neo-pentyl) or C₃₋₈ cycloalkyl (such as cyclohexyl). R⁶ can also be CH(CH₃)-cyclohexyl.

In another embodiment R⁶ is a C₃₋₁₂ cycloalkyl is unsubstituted or optionally substituted by one or more (eg one, two or three) substituents selected from halogen, C₁₋₆ alkyl, C₁₋₆ alkoxy, C₁₋₆ allylthio, C₁₋₆ alkylS(O), C₁₋₆ alkylS(O)₂, C₁₋₆ haloalkoxy, hydroxy, OC(O)(C₁₋₆ alkyl)).

In a further embodiment R⁶ is neo-pentyl, cyclopentyl, cyclohexyl, cycloheptyl, tetrahydropyranyl, tetrahydrothiopyranyl or adamantyl; the cyclic groups being optionally substituted by halogen, C₁₋₄ alkyl, C₁₋₄ alkoxy or hydroxy.

In yet another embodiment R⁶ is a C₃₋₁₂ cycloalkyl is unsubstituted or optionally substituted by one or more (eg one, two) halogen, C₁₋₃ alkyl (such as methyl)

In another embodiment R⁶ is cyclopentyl, cyclohexyl, 4,4-difluorocyclohexyl or cycloheptyl.

In a yet another embodiment R⁶ is CH(CH₃)₂, CH₂C(CH₃)₃, CH(CH₃)CH₂CH₃, CH(CH₃)CH(CH₃)₂ or CH(CH₃)(CH₂)₄CH₃.

In a further embodiment of the invention X and Z each independently represent a C₁₋₆, or C₁₋₄, or C₁₋₂ allylene group optionally substituted (e.g. none, one, two or three substituents) independently selected from halogen (e.g. fluorine, chlorine, bromine or iodine), trifluoromethyl, amino, (di)-C₁₋₆, or C₁₋₄, or C₁₋₂ alkylamino (e.g. methylamino, ethylamino, dimethylamino or diethylamino), (di)-C₁₋₆, or C₁₋₄, or C₁₋₂ alkylaminocarbonyl (e.g. methylaminocarbonyl, ethylaminocarbonyl, dimethylaminocarbonyl or diethylaminocarbonyl), C₁₋₆, or C₁₋₄, or C₁₋₂ alkylcarbonylamino (e.g. methylcarbonylamino or ethylcarbonylamino), sulphonamido or (di)-C₁₋₆, or C₁₋₄, or C₁₋₂ alkylaminosulphonyl (e.g. methylaminosulphonyl, ethylaminosulphonyl, dimethylaminsulphonyl or diethylaminsulphonyl).

In one embodiment, X represents a C₁₋₅ alkylene group.

In another embodiment E is CH₂CH₂, (CH₂)₃ or (CH₂)₄. In a further embodiment E is CH₂CH₂

In a still further embodiment k is 0.

In another embodiment of the invention k is 0; m is 1; and A is CH₂ or C(O).

In a further embodiment of the invention p is 1; k is 0; m is 1; and A is CH₂ or C(O).

In another embodiment, Z represents a C₁₋₂ alkylene group.

In an embodiment of the invention, p is 0 and q is 1.

In another embodiment, p is 1 and q is 0.

In still another embodiment, p and q are either both 0 or 1.

In a further embodiment, p and q are both 1.

In an embodiment of the invention, Y represents a bond, oxygen, CH₂ or NR⁹.

In a further embodiment of the invention R⁸ represents hydrogen or C₁₋₆, or C₁₋₄, or C₁₋₂ alkyl.

In an embodiment of the invention R⁹ represents hydrogen or C₁₋₆, or C₀₋₄, or C₁₋₂ alkyl.

In a further embodiment of the invention R¹⁰ represents hydrogen, or a saturated or unsaturated 3- to 12-membered (e.g. 3-, 4-, 5-, 6-, 7-, 8-, 9-, 10-, 11- or 12-membered) ring system optionally comprising at least one ring heteroatom (e.g. one, two, three or four ring heteroatoms independently) selected from nitrogen, oxygen and sulphur, the ring system being optionally substituted by halogen (e.g. fluorine, chlorine, bromine or iodine), trifluoromethyl, cyano, carboxy, hydroxy, nitro, —S(O)ᵣR¹⁵, —NR¹⁶S(O)_{S}R¹⁷, —C(O)NR¹⁸R¹⁹, —NHC(O)R²⁰, C₁₋₆, or C₁₋₄, or C₁₋₂ alkyl, C₁₋₆, or C₁₋₄, or C₁₋₂ alkoxy, C₁₋₆, or C₁₋₄, or C₁₋₂ alkylcarbonyl, C₁₋₆, or C₁₋₄, or C₁₋₂ alkoxycarbonyl and a saturated or unsaturated 4-, 5-, 6- or 7-membered monocyclic ring system optionally comprising at least one ring heteroatom (e.g. one, two, three or four ring heteroatoms independently) selected from nitrogen, oxygen and sulphur, the monocyclic ring system itself being optionally substituted by halogen (e.g. fluorine, chlorine, bromine or iodine), trifluoromethyl, hydroxy, —NR²¹S(O)ₜR²², —NHC(O)R²³ or C₁₋₆, or C₁₋₄, or C₁₋₂ alkoxy.

Examples of saturated or unsaturated 3- to 12-membered ring systems that may be used, which may be monocyclic or polycyclic (e.g. bicyclic or tricyclic) in which the two or more rings are fused, include one or more (in any combination) of cyclopropyl, cyclobutyl, cyclopentyl, cyclohexyl, cycloheptyl, adamantyl, bicyclo[2.2.1]heptyl, cyclopentenyl, cyclohexenyl, phenyl, pyrrolidinyl, piperidinyl, piperazinyl, morpholinyl, thiomorpholinyl, diazabicyclo[2.2.1]hept-2-yl, naphthyl, benzofuranyl, benzothienyl, benzodioxolyl, quinolinyl, oxazolyl, 2,3-dihydrobenzofuranyl, tetrahydropyranyl, pyrazolyl, pyrazinyl, thiazolidinyl, indanyl, thienyl, isoxazolyl, pyridazinyl, thiadiazolyl, pyrrolyl, furanyl, thiazolyl, indolyl, imidazolyl, pyrimidinyl, benzimidazolyl, triazolyl, tetrazolyl and pyridinyl.

Examples of saturated or unsaturated 4- to 7-membered monocyclic ring systems that may be used include cyclobutyl, cyclopentyl, cyclohexyl, pyrrolidinyl, piperazinyl, morpholinyl, furanyl, thienyl, pyrrolyl, phenyl, oxazolyl, 1,2,4-oxadiazolyl, 1,3,4-oxadiazolyl, isoxazolyl, imidazolyl, pyrazolyl, thiazolyl, triazolyl, thiadiazolyl, pyridinyl, pyrimidinyl, pyrazinyl and tetrazolyl.

In an embodiment of the invention, R¹⁰ represents hydrogen, or a saturated or unsaturated 5- or 6-membered ring system optionally comprising at least one ring heteroatom (e.g. one or two ring heteroatoms independently) selected from nitrogen and oxygen, the ring system being optionally substituted by halogen (e.g. fluorine, chlorine, bromine or iodine), trifluoromethyl, cyano, carboxyl, hydroxyl, nitro, —S(O)ᵣR¹⁵, —NR¹⁶S(O)SR¹⁷, —C(O)NR¹⁸R¹⁹, —NHC(O)R²⁰, C₁₋₆, or C₁₋₄, or C₁₋₂ alkyl, C₁₋₆, or C₁₋₄, or C₁₋₂ alkoxy, C₁₋₆, or C₁₋₄, or C₁₋₂ alkylcarbonyl, C₁₋₆, or C₁₋₄, or C₁₋₂ alkoxycarbonyl or a saturated or unsaturated 4-, 5-, 6- or 7-membered monocyclic ring system optionally comprising at least one ring heteroatom (e.g. one, two, three or four ring heteroatoms) independently selected from nitrogen, oxygen and sulphur, the monocyclic ring system itself being optionally substituted by halogen (e.g. fluorine, chlorine, bromine or iodine), trifluoromethyl, hydroxyl, —NR²¹S(O)ₜR²², —NHC(O)R²³ or C₁₋₆, or C₁₋₄, or C₁₋₂ alkoxy.

In another embodiment, R¹⁰ represents hydrogen, or a saturated or unsaturated 5- or 6-membered ring system optionally comprising at least one ring heteroatom (e.g. one or two ring heteroatoms independently) selected from nitrogen and oxygen, the ring system being optionally substituted by halogen (e.g. fluorine, chlorine, bromine or iodine), trifluoromethyl, cyano, carboxy, hydroxy, nitro, —S(O)ᵣR¹⁵, —NR¹⁶S(O)_{S}R¹⁷, —C(O)NR¹⁸R¹⁹, —NHC(O)R²⁰, C₁₋₄ or C₁₋₂ alkyl, C₁₋₄ or C₁₋₂ alkoxy, C₁₋₄ or C₁₋₂ alkylcarbonyl, C₁₋₄ or C₁₋₂ alkoxycarbonyl or a saturated or unsaturated 5- or 6-membered monocyclic ring system optionally comprising at least one ring heteroatom (e.g. one or two ring heteroatoms independently) selected from nitrogen, oxygen and sulphur, the monocyclic ring system itself being optionally substituted by halogen (e.g. fluorine, chlorine, bromine or iodine), trifluoromethyl, hydroxy, —NR²¹S(O)ₜR²², —NHC(O)R²³ or C₁₋₄ or C₁₋₂ alkoxy.

In a further embodiment, R¹⁰ represents hydrogen, or a saturated or unsaturated 5- or 6-membered ring system optionally comprising one or two ring heteroatoms independently selected from nitrogen and oxygen, the ring system being optionally substituted by halogen (e.g. fluorine, chlorine, bromine or iodine), trifluoromethyl, carboxyl, hydroxyl, —S(O)ᵣR¹⁵, —NR¹⁶S(O)_{S}R¹⁷, —C(O)NR¹⁸R¹⁹, —NHC(O)R²⁰, C₁₋₄ or C₁₋₂ alkyl, C₁₋₄ or C₁₋₂ alkoxy, C₁₋₄ or C₁₋₂ alkylcarbonyl or C₁₋₄ or C₁₋₂ alkoxycarbonyl.

In a still further embodiment, R¹⁰ represents hydrogen, or a saturated or unsaturated 5- or 6-membered ring system optionally comprising one or two ring heteroatoms independently selected from nitrogen and oxygen, the ring system being optionally substituted by C₁₋₄ or C₁₋₂ alkoxycarbonyl.

In a further embodiment of the invention R¹⁵, R¹⁶, R¹⁷, R¹⁸, R¹⁹, R²⁰, R²¹, R²², and R²³ each independently represent hydrogen or C₁₋₆, or C₁₋₄, or C₁₋₂ alkyl.

In another embodiment of the invention r, s and t are all 2.

In a further embodiment R⁶ is a group —(X)ₚ—Y—(Z)_{q}—R¹⁰ wherein: p and q are both 1; X and Z are, independently, C₁₋₆ alkylene; R¹⁰ is as defined above (for example it is hydrogen); Y is NR⁹; and R⁹ is C₁₋₆ alkyl.

In a still further embodiment R⁶ is a group —(X)ₚ—Y—(Z)_{q}—R¹⁰ wherein: p and q are both 0; Y is a bond and R¹⁰ is a saturated or unsaturated 3- to 12-membered ring system optionally comprising a ring heteroatom selected from nitrogen, oxygen and sulphur, the ring system being optionally substituted by halogen, trifluoromethyl, cyano, carboxyl, hydroxy, —S(O)ᵣR¹⁵, —NR¹⁶S(O)ₛR¹⁷—C(O)NR¹⁸R¹⁹, —NHC(O)R²⁰, C₁₋₆ alkyl, C₁₋₆ alkoxy, C₁₋₆ alkylcarbonyl, C₁₋₆ alkoxycarbonyl or a saturated or unsaturated 4- to 7-membered monocyclic ring system optionally comprising at least one ring heteroatom selected from nitrogen, oxygen and sulphur, the monocyclic ring system itself being optionally substituted by halogen, trifluoromethyl, hydroxy, —NR²¹S(O)ₜR²², —NHC(O)R²³ or C₁₋₆ alkoxy; R¹⁵, R¹⁶, R¹⁷, R¹⁸, R¹⁹, R²⁰, R²¹, R²² and R²³ each independently represent hydrogen or C₁₋₆ alkyl; and r, s and t are all 2.

In a further embodiment R⁶ is a group —(X)ₚ—Y—(Z)_{q}—R¹⁰ wherein: p and q are both 0; Y is a bond and R¹⁰ is a saturated or unsaturated 3- to 12-membered ring system (such as cyclopentyl, cyclohexyl, cycloheptyl, adamantyl, bicyclo[2.2.1]heptyl, pyrrolidinyl, piperidinyl, piperazinyl, morpholinyl, thiomorpholinyl or diazabicyclo[2.2.1]hept-2-yl) optionally comprising a ring heteroatom selected from nitrogen, oxygen and sulphur, the ring system being optionally substituted by halogen, trifluoromethyl, cyano, carboxyl, hydroxy, —S(O)ᵣR¹⁵, —NR¹⁶S(O)_{S}R¹⁷, —C(O)NR¹⁸R¹⁹, —NHC(O)R²⁰, C₁₋₆ alkyl, C₁₋₆ alkoxy, C₁₋₆ alkylcarbonyl or C₁₋₆ alkoxycarbonyl; R¹⁵, R¹⁶, R¹⁷, R¹⁸, R¹⁹ and R²⁰ each independently represent hydrogen or C₁₋₆ alkyl; and r and s are both 2.

In an embodiment of the invention R⁷ represents a 5- to 14-membered (5-, 6-, 7-, 8-, 9-, 10-, 11-, 12-, 13- or 14-membered) aromatic or heteroaromatic ring system optionally substituted by halogen (e.g. fluorine, chlorine, bromine or iodine), trifluoromethyl, hydroxyl, carboxyl, C₁₋₆, or C₁₋₄, or C₁₋₂ alkyl (optionally substituted by —NR²⁴R²⁵), C₁₋₆, or C₁₋₄, or C₁₋₂ alkoxy (optionally substituted by —NR²⁶R²⁷), C₁₋₆, or C₁₋₄, or C₁₋₂ alkoxycarbonyl, —NR²⁸R²⁹, C₁₋₆, or C₁₋₄, or C₁₋₂ alkylcarbonylamino, C₁₋₆, or C₁₋₄, or C₁₋₂ alkylsulphonylamino, phenylsulphonylamino, —C(O)NHR³⁰, —SO₂NHR³³, C₀₋₆, or C₀₋₄, or C₀₋₂ alkyl-R³⁴, or phenyl or 5- to 6-membered heteroaromatic ring (each of which is optionally substituted by halogen such as fluorine, chlorine, bromine or iodine, trifluoromethyl, hydroxyl, C₁₋₆, or C₁₋₄, or C₁₋₂ alkyl, C₁₋₆, or C₁₋₄, or C₁₋₂ alkoxy or —NR³⁵R³⁶).

When R⁷ represents an optionally substituted 5- to 14-membered heteroaromatic ring system, the ring system comprises, for example, from 1 to 4 ring heteroatoms independently selected from nitrogen, oxygen and sulphur. Similarly, if a substituent in R⁷ represents an optionally substituted 5- to 6-membered heteroaromatic ring, the ring comprises from 1 to 4 ring heteroatoms independently selected from nitrogen, oxygen and sulphur.

Examples of 6- to 14-membered aromatic or heteroaromatic ring systems that may be used, which may be monocyclic or polycyclic (e.g. bicyclic or tricyclic) in which the two or more rings are fused, include one or more (in any combination) of phenyl, naphthyl, pyridinyl, pyridazinyl, pyrimidinyl, pyrazinyl, 1,3,5-triazinyl, 1,2,4-triazinyl, azepinyl, is oxepinyl, thiepinyl, indenyl, benzofuranyl, isobenzofuranyl, benzothiophenyl, indolyl, isoindolyl, benzimidazolyl, indazolyl, benzisoxazolyl, benzoxazolyl, benzothiazolyl, quinolinyl, isoquinolinyl, quinazolinyl, quinoxalinyl and dibenzofuranyl. Preferred ring systems include phenyl and naphthyl.

Examples of 5- to 6-membered heteroaromatic rings include pyridinyl, triazolyl and tetrazolyl.

In an embodiment of the invention, R⁷ represents a 5- to 10-membered aromatic or heteroaromatic ring system optionally substituted by halogen (e.g. fluorine, chlorine, bromine or iodine), trifluoromethyl, hydroxyl, carboxyl, C₁₋₄, or C₁₋₂ alkyl (optionally substituted by —NR²⁴R²⁵), C₁₋₄, or C₁₋₂ alkoxy (optionally substituted by —NR²⁶R²⁷), C₁₋₄, or C₁₋₂ alkoxycarbonyl, —NR²⁸R²⁹, C₁₋₄, or C₁₋₂ alkylcarbonylamino, C₁₋₄, or C₁₋₂ alkylsulphonylamino, phenylsulphonylamino, —C(O)NHR³⁰, —SO₂NHR³³, C₀₋₄ or C₀₋₂ alkyl-R³⁴, phenyl or a 5- to 6-membered heteroaromatic ring.

In another embodiment, R⁷ represents a 6- to 10-membered aromatic ring system unsubstituted or optionally substituted by one or more (eg one, two, three or four) substituents independently selected from halogen (e.g. fluorine, chlorine, bromine or iodine), trifluoromethyl, hydroxyl, carboxyl, C₁₋₄, or C₁₋₂ alkyl (optionally substituted by at least one, e.g. one or two, —NR²⁴R²⁵), C₁₋₄, or C₁₋₂ alkoxy (optionally substituted by at least one, e.g. one or two, —NR²⁶R²⁷), C₁₋₄, or C₁₋₂ alkloxycarbonyl, —NR²⁸R²⁹C₁₋₄, or C₁₋₂ alkylcarbonylamino, C₁₋₄, or C₁₋₂ alkylsulphonylamino, phenylsulphonylamino, —C(O)NHR³⁰, —SO₂NHR³³, C₀₋₄ or C₀₋₂ alkyl-R³⁴, phenyl and a 5- to 6-membered heteroaromatic ring.

In yet another embodiment, R⁷ is a 6- to 10-membered aromatic or heteroaromatic ring system (such as phenyl, thienyl, pyridinyl or pyrimidinyl) unsubstituted or optionally substituted by one or more (eg one, two, three or four) substituents independently selected from halogen (e.g. fluorine, chlorine, bromine or iodine), trifluoromethyl, hydroxyl, carboxyl, C₁₋₄ alkyl (optionally substituted by —NR²⁴R²⁵), C₁₋₄ alkoxy (optionally substituted by —NR²⁶R²⁷), C₁₋₄ alkoxycarbonyl, —NR²⁸R²⁹, C₁₋₄ alkylcarbonylamino, C₁₋₄ alkylsulphonylamino, —C(O)NHR³⁰ or —SO₂NHR³³; and R²⁴, R²⁵, R²⁶, R²⁷, R²⁸, R²⁹, R³⁰ and R³³ are, independently, hydrogen or C₁₋₆ alkyl.

In a further embodiment the present invention provides a compound of formula (I) wherein R⁷ is thienyl optionally substituted by halogen (such as chloro); or R⁷ is phenyl optionally substituted by halogen (such as chloro or fluoro), C₁₋₄ alkyl (such as methyl), C₁₋₄ alkoxy (such as methoxy or ethoxy), hydroxy, cyano, CO₂H or phenyl.

In a still further embodiment R⁷ is phenyl optionally substituted by (for example unsubstituted or substituted by one, two of three of the same or different) halogen (such as fluoro or chloro), C₁₋₄ alkyl (such as methyl), hydroxy, cyano or C₁₋₄ alkoxy.

In an embodiment of the invention R²⁴, R²⁵, R²⁶, R²⁷, R²⁸ and R²⁹ each independently represent hydrogen or C₁₋₆, or C₁₋₄, or C₁₋₂ alkyl. It should be understood that if there is more than one group —NR²⁴R²⁵, the groups may be the same as, or different from, one another. Similar comments apply if there is more than one group —NR²⁶R²⁷.

In a further embodiment of the invention R³⁰ represents hydrogen; C₁₋₆, or C₁₋₄, or C₁₋₂ alkyl; phenyl-C₀₋₆, or C₀₋₄, or C₀₋₂ alkyl (e.g. phenyl or benzyl); or C₂₋₆ or C₂₋₄ allylene —NR³¹R³² and either R³¹ and R³² each independently represent hydrogen or C₁₋₆, or C₁₋₄, or C₁₋₂ alkyl, or R³¹ and R³² together with the nitrogen atom to which they are attached form a 4- to 6-membered saturated heterocyclic ring optionally comprising a further ring heteroatom selected from nitrogen and oxygen such as azetidinyl, pyrrolidinyl, piperidinyl, piperazinyl or morpholinyl.

In an embodiment of the invention R³³ represents hydrogen; C₁₋₆, or C₁₋₄, or C₁₋₂ alkyl; phenyl-C₀₋₆, or C₀₋₄, or C₀₋₂ alkyl (e.g. phenyl or benzyl); or C₂₋₆ or C₂₋₄ alkylene-NR³⁷R³⁸ and either R³⁷ and R³⁸ each independently represent hydrogen or C₁₋₆, or C₁₋₄, or C₁₋₂ alkyl, or R³⁷ and R³⁸ together with the nitrogen atom to which they are attached form a 4- to 6-membered saturated heterocyclic ring optionally comprising a further ring heteroatom selected from nitrogen and oxygen such as azetidinyl, pyrrolidinyl, piperidinyl, piperazinyl or morpholinyl.

In another embodiment of the invention R³⁴ represents a saturated, 5- or 6-membered nitrogen-containing ring, e.g. a ring containing one or two ring nitrogen atoms such as hydantoin.

In a further embodiment of the invention R³⁵ and R³⁶ each independently represent hydrogen or C₁₋₆, or C₁₋₄, or C₁₋₂ alkyl.

In an embodiment of the invention (subject to the proviso hereinbefore defined),

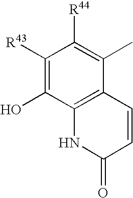

Ar is
k is 0 or 1 (for example 0);
each of R², R³, R⁴, R⁵, R^{4′} and R^{5′} represents hydrogen;
A represents C(O) or CH₂ (for example A is C(O));
D represents oxygen;
m is 1;
R⁶ represents C₁₋₆ alkyl;
R⁷ represents a 6- to 10-membered aromatic ring system optionally substituted by one or more halogen atoms (the ring system is, for example, phenyl; and R⁷ is, for example, unsubstituted phenyl).

In a further embodiment the present invention provides a compound of formula (I) wherein:

### Ar is:

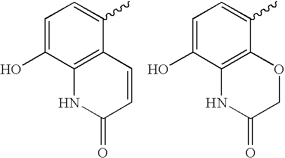

k is 0;
m is 1;

### D is O or NH;

### A is C(O);

R², R³, R⁴ and R⁵ are all hydrogen;

### E is CH₂CH₂;

R⁶ is cyclopentyl, cyclohexyl, 4,4-difluorocyclohexyl, cycloheptyl, or an α- or β-branched C₃₋₁₂ alkyl (for example CH(CH₃)₂, CH₂C(CH₃)₃, CH(CH₃)CH₂CH₃, CH(CH₃)CH(CH₃)₂ or CH(CH₃)(CH₂)₄CH₃);
R⁷ is thienyl optionally substituted by halogen (such as chloro); or R⁷ is phenyl optionally substituted by halogen (such as chloro or fluoro), C₁₋₄ alkyl (such as methyl), C₁₋₄ alkoxy (such as methoxy or ethoxy), hydroxy or cyano;
or a pharmaceutically acceptable salt thereof.

The ring system of R⁷ is optionally substituted and in a further aspect of the invention the ring system is unsubstituted or mono- or di-substituted.

A suitable pharmaceutically acceptable salt is, for example, an acid addition salt such as a hydrochloride (for example a monohydrochloride or a dihydrochloride), hydrobromide (for is example a monohydrobromide or a dihydrobromide), trifluoroacetate (for example a mono-trifluoroacetate or a di-trifluoroacetate), sulphate, phosphate, acetate, fumarate, maleate, tartrate, lactate, citrate, pyruvate, succinate, oxalate, methanesulplhonate, p-toluenesulphonate, bisulphate, benzenesulphonate, ethanesulphonate, malonate, xinafoate, ascorbate, oleate, nicotinate, saccharinate, adipate, formate, glycolate, L-lactate, D-lactate, aspartate, malate, L-tartrate, D-tartrate, stearate, 2-furoate, 3-furoate, napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isethionate (2-hydroxyethylsulfonate), 2-mesitylenesulphonate, 2-naphthalenesulphonate, D-mandelate, L-mandelate, 2,5-dichlorobenzenesulphonate, cinnamate or benzoate. In another aspect of the invention the stoichiometry of the salt is, for example, a hemi-salt, or a mono- or di-salt.

Examples of compounds of formula (I) include:
N-Butyl-N-(2-(2-(8-hydroxy-2-oxo-1,2-dihydroquinolin-5-yl)ethylamino)ethyl)-3-phenethoxypropanamide;
N-cyclohexyl-N-(2-(3-formamido-4-hydroxyphenethylamino)ethyl)-3-(phenethylamino)propanamide;
N-cyclohexyl-N-[2-({2-[4-hydroxy-3-(hydroxymethyl)phenyl]ethyl}amino)ethyl]-N³-(2-phenylethyl)-β-alaninamide;
N-cyclohexyl-N-(2-{[2-(8-hydroxy-2-oxo-1,2-dihydroquinolin-5-yl)ethyl]amino}ethyl)-N³-(2-phenylethyl)-β-alaninamide;
N³-[2-(3-chlorophenyl)ethyl]-N-cyclohexyl-N-[2-({2-[3-(formylamino)-4-hydroxyphenyl]ethyl}amino)ethyl]-β-alaninamide;
N-cyclohexyl-N³-[2-(3-fluorophenyl)ethyl]-N-[2-({2-[3-(formylamino)-4-hydroxyphenyl]ethyl}amino)ethyl]-β-alaninamide;
N-cyclohexyl-N-[2-({2-[3-(formylamino)-4-hydroxyphenyl]ethyl}amino)ethyl]-N³-[2-(3-methylphenyl)ethyl]-β-alaninamide;
N³-[2-(3-chlorophenyl)ethyl]-N-cyclohexyl-N-[2-({2-[4-hydroxy-3-(hydroxymethyl)phenyl]ethyl}amino)ethyl]-β-alaninamide;
N-cyclohexyl-N³-[2-(3-fluorophenyl)ethyl]-N-[2-({2-[4-hydroxy-3-(hydroxymethyl)phenyl]ethyl}amino)ethyl]-β-alaninamide;
N-cyclohexyl-N-[2-({2-[4-hydroxy-3-(hydroxymethyl)phenyl]ethyl}amino)ethyl]-N³-[2-(3-methylphenyl)ethyl]-β-alaninamide;
3-(3-Chlorophenethylamino)-N-cyclohexyl-N-(2-(2-(8-hydroxy-2-oxo-1,2-dihydroquinolin-5-yl)ethylamino)ethyl)propanamide;
N-Cyclohexyl-3-(3-fluorophenethylamino)-N-(2-(2-(8-hydroxy-2-oxo-1,2-dihydroquinolin-5-yl)ethylamino)ethyl)propanamide;
N-cyclohexyl-N-(2-{[2-(8-hydroxy-2-oxo-1,2-dihydroquinolin-5-yl)ethyl]amino}ethyl)-N³-[2-(3-methylphenyl)ethyl]-β-alaninamide;
N-(2,2-dimethylpropyl)-N-[2-({2-[3-(formylamino)-4-hydroxyphenyl]ethyl}amino)ethyl]-N³-(2-phenylethyl)-β-alaninamide;
N-(2,2-dimethylpropyl)-N-[2-({2-[4-hydroxy-3-(hydroxymethyl)phenyl]ethyl}amino)ethyl]-N³-(2-phenylethyl)-β-alaninamide;
N-(2,2-dimethylpropyl)-N-(2-{[2-(8-hydroxy-2-oxo-1,2-dihydroquinolin-5-yl)ethyl]amino}ethyl)-N³-(2-phenylethyl)-β-alaninamide;
N³-[2-(3-chlorophenyl)ethyl]-N-(2,2-dimethylpropyl)-N-[2-({2-[3-(formylamino)-4-hydroxyphenyl]ethyl}amino)ethyl]-β-alaninamide;
N-(2,2-dimethylpropyl)-N³-[2-(3-fluorophenyl)ethyl]-N-[2-({2-[3-(formylamino)-4-hydroxyphenyl]ethyl}amino)ethyl]-β-alaninamide;
N-(2,2-dimethylpropyl)-N-[2-({2-[3-(formylamino)-4-hydroxyphenyl]ethyl}amino)ethyl]-N³-[2-(3-methylphenyl)ethyl]-β-alaninamide;
N³-[2-(3-chlorophenyl)ethyl]-N-(2,2-dimethylpropyl)-N-[2-({2-[4-hydroxy-3-(hydroxymethyl)phenyl]ethyl}amino)ethyl]-β-alaninamide;
N-(2,2-dimethylpropyl)-N³-[2-(3-fluorophenyl)ethyl]-N-[2-({2-[4-hydroxy-3-(hydroxymethyl)phenyl]ethyl}amino)ethyl]-β-alaninamide;
N-(2,2-dimethylpropyl)-N-[2-({2-[4-hydroxy-3-(hydroxymethyl)phenyl]ethyl}amino)ethyl]-N³-[2-(3-methylphenyl)ethyl]-β-alaninamide;
N³-[2-(3-chlorophenyl)ethyl]-N-(2,2-dimethylpropyl)-N-(2-{[2-(8-hydroxy-2-oxo-1,2-dihydroquinolin-5-yl)ethyl]amino}ethyl)-β-alaninamide;
N-(2,2-dimethylpropyl)-N³-[2-(3-fluorophenyl)ethyl]-N-(2-{[2-(8-hydroxy-2-oxo-1,2-dihydroquinolin-5-yl)ethyl]amino}ethyl)-β-alaninamide;
N-(2,2-dimethylpropyl)-N-(2-{[2-(8-hydroxy-2-oxo-1,2-dihydroquinolin-5-yl)ethyl]amino}ethyl)-N³-[2-(3-methylphenyl)ethyl]-β-alaninamide;
N-cyclohexyl-N-(2-(2-(5-hydroxy-3-oxo-3,4-dihydro-2H-benzo[b][1,4]oxazin-8-yl)ethylamino)ethyl)-3-(phenethylamino)propanamide;
N-cyclohexyl-N-(2-{[2-(6-hydroxy-3-oxo-3,4-dihydro-2H-1,4-benzoxazin-8-yl)ethyl]amino}ethyl)-N-3-(2-phenylethyl)-β-alaninamide;
N-(2,2-dimethylpropyl)-N-(2-{[2-(5-hydroxy-3-oxo-3,4-dihydro-2H-1,4-benzoxazin-8-yl)ethyl]amino}ethyl)-N³-(2-phenylethyl)-β-alaninamide; or,
N-(2,2-dimethylpropyl)-N-(2-{[2-(6-hydroxy-3-oxo-3,4-dihydro-2H-1,4-benzoxazin-8-yl)ethyl]amino}ethyl)-N³-(2-phenylethyl)-β-alaninamide;
   or a pharmaceutically acceptable salt thereof.

Each individual compound of the Examples, which can be in the form of a free base (in the case where the Example is a salt).

In a further aspect the present invention provides a compound of formula (I) which is:
N-Butyl-N-(2-(2-(8-hydroxy-2-oxo-1,2-dihydroquinolin-5-yl)ethylamino)ethyl)-3-phenethoxypropanamide;
N-Cyclohexyl-N-(2-(2-(8-hydroxy-2-oxo-1,2-dihydroquinolin-5-yl)ethylamino)ethyl)-3-phenethoxypropanamide;
3-(3-Chlorophenethylamino)-N-cycloheptyl-N-(2-(2-(8-hydroxy-2-oxo-1,2-dihydroquinolin-5-yl)ethylamino)ethyl)propanamide;
3-(4-Chlorophenethylamino)-N-cycloheptyl-N-(2-(2-(8-hydroxy-2-oxo-1,2-dihydroquinolin-5-yl)ethylamino)ethyl)propanamide;
N-Cycloheptyl-3-(3-fluorophenethylamino)-N-(2-(2-(8-hydroxy-2-oxo-1,2-dihydroquinolin-5-yl)ethylamino)ethyl)propanamide;
N-Cycloheptyl-3-(4-fluorophenethylamino)-N-(2-(2-(8-hydroxy-2-oxo-1,2-dihydroquinolin-5-yl)ethylamino)ethyl)propanamide;
N-Cycloheptyl-N-(2-(2-(8-hydroxy-2-oxo-1,2-dihydroquinolin-5-yl)ethylamino)ethyl)-3-(phenethylamino)propanamide;
N-Cycloheptyl-N-(2-(2-(8-hydroxy-2-oxo-1,2-dihydroquinolin-5-yl)ethylamino)ethyl)-3-(3-methylphenethylamino)propanamide;
(S)—N-Cycloheptyl-N-(2-(2-(8-hydroxy-2-oxo-1,2-dihydroquinolin-5-yl)ethylamino)ethyl)-3-(2-phenylpropylamino)propanamide;
N-Cycloheptyl-3-(3,5-difluorophenethylamino)-N-(2-(2-(8-hydroxy-2-oxo-1,2-dihydroquinolin-5-yl)ethylamino)ethyl)propanamide;
N-Cycloheptyl-3-(3-ethoxyphenethylamino)-N-(2-(2-(8-hydroxy-2-oxo-1,2-dihydroquinolin-5-yl)ethylamino)ethyl)propanamide;
3-(2-(Biphenyl-3-yl)ethylamino)-N-cycloheptyl-N-(2-(2-(8-hydroxy-2-oxo-1,2-dihydroquinolin-5-yl)ethylamino)ethyl)propanamide;
3-(3-Chlorophenethylamino)-N-cyclohexyl-N-(2-(2-(8-hydroxy-2-oxo-1,2-dihydroquinolin-5-yl)ethylamino)ethyl)propanamide;
3-(4-Chlorophenethylamino)-N-cyclohexyl-N-(2-(2-(8-hydroxy-2-oxo-1,2-dihydroquinolin-5-yl)ethylamino)ethyl)propanamide;
N-Cyclohexyl-3-(3,5-difluorophenethylamino)-N-(2-(2-(8-hydroxy-2-oxo-1,2-dihydroquinolin-5-yl)ethylamino)ethyl)propanamide;
N-Cyclohexyl-3-(3,4-difluorophenethylamino)-N-(2-(2-(8-hydroxy-2-oxo-1,2-dihydroquinolin-5-yl)ethylamino)ethyl)propanamide;
N-Cyclohexyl-3-(2,5-difluorophenethylamino)-N-(2-(2-(8-hydroxy-2-oxo-1,2-dihydroquinolin-5-yl)ethylamino)ethyl)propanamide;
N-Cyclohexyl-3-(2,3-difluorophenethylamino)-N-(2-(2-(8-hydroxy-2-oxo-1,2-dihydroquinolin-5-yl)ethylamino)ethyl)propanamide;
N-Cyclohexyl-3-(3-fluorophenethylamino)-N-(2-(2-(8-hydroxy-2-oxo-1,2-dihydroquinolin-5-yl)ethylamino)ethyl)propanamide;
N-Cyclohexyl-3-(3-fluorophenethylamino)-N-(2-(2-(7-hydroxy-2-oxoindolin-4-yl)ethylamino)ethyl)propanamide;
N-Cyclohexyl-N-(2-(3-formamido-4-hydroxyphenethylamino)ethyl)-3-(phenethylamino)propanamide;
N-Cyclohexyl-N-(2-(3,4-dihydroxyphenethylamino)ethyl)-3-(phenethylamino)propanamide;
N-Cyclohexyl-N-(2-(2-(5-hydroxy-3-oxo-3,4-dihydro-2H-benzo[b][1,4]oxazin-8-yl)ethylamino)ethyl)-3-(phenethylamino)propanamide;
N-Cycloheptyl-N-(2-(2-(5-hydroxy-3-oxo-3,4-dihydro-2H-benzo[b][1,4]oxazin-8-yl)ethylamino)ethyl)-3-(phenethylamino)propanamide;
(R)—N-(Hexan-2-yl)-N-(2-(2-(5-hydroxy-3-oxo-3,4-dihydro-2H-benzo[b][1,4]oxazin-8-yl)ethylamino)ethyl)-3-(phenethylamino)propanamide;
N-Cycloheptyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide;
3-(3-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
3-(2-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(2,3-dichlorophenethylamino)-N-(2-(2-(5-hydroxy-3-oxo-3,4-dihydro-2H-benzo[b][1,4]oxazin-8-yl)ethylamino)ethyl)propanamide;
N-Cyclohexyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide;
N-Cyclohexyl-3-(2,4-dichlorophenethylamino)-N-(2-(2-(5-hydroxy-3-oxo-3,4-dihydro-2H-benzo[b][1,4]oxazin-8-yl)ethylamino)ethyl)propanamide;
3-(4-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(2-fluoro-5-methylphenethylamino)-N-(2-(2-(5-hydroxy-3-oxo-3,4-dihydro-2H-benzo[b][1,4]oxazin-8-yl)ethylamino)ethyl)propanamide;
3-(5-Chloro-2-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(3,5-difluorophenethylamino)-N-(2-(2-(5-hydroxy-3-oxo-3,4-dihydro-2H-benzo[b][1,4]oxazin-8-yl)ethylamino)ethyl)propanamide;
N-Cyclohexyl-3-(2,3-difluorophenethylamino)-N-(2-(2-(5-hydroxy-3-oxo-3,4-dihydro-2H-benzo[b][1,4]oxazin-8-yl)ethylamino)ethyl)propanamide;
3-(3-Chloro-4-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(2,5-difluorophenethylamino)-N-(2-(2-(5-hydroxy-3-oxo-3,4-dihydro-2H-benzo[b][1,4]oxazin-8-yl)ethylamino)ethyl)propanamide;
3-(3-Chloro-2-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(4-fluoro-3-methylphenethylamino)-N-(2-(2-(5-hydroxy-3-oxo-3,4-dihydro-2H-benzo[b][1,4]oxazin-8-yl)ethylamino)ethyl)propanamide;
3-(3-Chloro-5-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(3,4-difluorophenethylamino)-N-(2-(2-(5-hydroxy-3-oxo-3,4-dihydro-2H-benzo[b][1,4]oxazin-8-yl)ethylamino)ethyl)propanamide;
4-(2-(3-(Cyclohexyl(2-(2-(5-hydroxy-3-oxo-3,4-dihydro-2H-benzo[b][1,4]oxazin-8-yl)ethylamino)ethyl)amino)-3-oxopropylamino)ethyl)benzoic acid;
(R)-3-(3-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanalnide;
(R)-3-(4-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(4-Fluoro-3-methylphenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3-Chloro-4-fluorophenethylamino)-N-(hexaxo-2-yl)-N-(2-(2-(5-hydroxy-3-oxo-3,4-dihydro-2H-benzo[b][1,4]oxazin-8-yl)ethylamino)ethyl)propanamide;
(R)-3-(2-(5-Chlorothiophen-2-yl)ethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3-Chloro-5-fluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3,5-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3,4-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2,3-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2,5-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3,4-Dichlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2-Fluoro-5-methylphenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(5-Fluoro-2-methylplenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)—N-sec-Butyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide;
(R)-3-(3,4-Dichlorophenethylamino)-N-(2-(2-(5-hydroxy-3-oxo-3,4-dihydro-2H-benzo[b][1,4]oxazin-8-yl)ethylamino)ethyl)-N-(3-methylbutan-2-yl)propanamide;
(R)—N-(Heptan-2-yl)-N-(2-(2-(5-hydroxy-3-oxo-3,4-dihydro-2H-benzo[b][1,4]oxazin-8-yl)ethylamino)ethyl)-3-(phenethylamino)propanamide;
(R)—N-(1-Cyclohexylethyl)-N-(2-(2-(5-hydroxy-3-oxo-3,4-dihydro-2H-benzo[b][1,4]oxazin-8-yl)ethylamino)ethyl)-3-(phenethylamino)propanamide;
N-Cycloheptyl-3-(4-fluoro-3-methylphenethylamino)-N-(2-(2-(5-hydroxy-3-oxo-3,4-dihydro-2H-benzo[b][1,4]oxazin-8-yl)ethylamino)ethyl)propanamide;
3-(4-Chlorophenethylamino)-N-cycloheptyl-N-(2-(2-(5-hydroxy-3-oxo-3,4-dihydro-2H-benzo[b][1,4]oxazin-8-yl)ethylamino)ethyl)propanamide;
N-Cycloheptyl-3-(2,5-difluorophenethylamino)-N-(2-(2-(5-hydroxy-3-oxo-3,4-dihydro-2H-benzo[b][1,4]oxazin-8-yl)ethylamino)ethyl)propanamide;
N-Cycloheptyl-3-(3,5-difluorophenethylamino)-N-(2-(2-(5-hydroxy-3-oxo-3,4-dihydro-2H-benzo[b][1,4]oxazin-8-yl)ethylamino)ethyl)propanamide;
3-(3-Chloro-5-fluorophenethylamino)-N-cycloheptyl-N-(2-(2-(5-hydroxy-3-oxo-3,4-dihydro-2H-benzo[b][1,4]oxazin-8-yl)ethylamino)ethyl)propanamide;
N-Cycloheptyl-3-(3,4-difluorophenethylamino)-N-(2-(2-(5-hydroxy-3-oxo-3,4-dihydro-2H-benzo[b][1,4]oxazin-8-yl)ethylamino)ethyl)propanamide;
3-(3-Chlorophenethylamino)-N-Cycloheptyl-N-(2-(2-(5-hydroxy-3-oxo-3,4-dihydro-2H-benzo[b][1,4]oxazin-8-yl)ethylamino)ethyl)propanamide;
N-Cycloheptyl-3-(3-fluorophenethylamino)-N-(2-(2-(5-hydroxy-3-oxo-3,4-dihydro-2H-benzo[b][1,4]oxazin-8-yl)ethylamino)ethyl)propanamide;
N-Cyclohexyl-N-(2-(2-(5-hydroxy-3-oxo-3,4-dihydro-2H-benzo[b][1,4]oxazin-8-yl)ethylamino)ethyl)-3-phenethoxypropanamide;
N-Cyclohexyl-3-(4-fluorophenethoxy)-N-(2-(2-(8-hydroxy-2-oxo-1,2-dihydroquinolin-5-yl)ethylamino)ethyl)propanamide;
N-Cyclohexyl-3-(3-fluorophenethoxy)-N-(2-(2-(8-hydroxy-2-oxo-1,2-dihydroquinolin-5-yl)ethylamino)ethyl)propanamide;
N-Cyclohexyl-3-(2-fluorophenethoxy)-N-(2-(2-(8-hydroxy-2-oxo-1,2-dihydroquinolin-5-yl)ethylamino)ethyl)propanamide;
N-Cyclohexyl-3-(3-fluorophenethoxy)-N-(2-(2-(5-hydroxy-3-oxo-3,4-dihydro-2H-benzo[b][1,4]oxazin-8-yl)ethylamino)ethyl)propanamide;
N-Cyclohexyl-3-(4-fluorophenethoxy)-N-(2-(2-(5-hydroxy-3-oxo-3,4-dihydro-2H-benzo[b][1,4]oxazin-8-yl)ethylamino)ethyl)propanamide;
N-Cyclohexyl-3-(2-fluorophenethoxy)-N-(2-(2-(5-hydroxy-3-oxo-3,4-dihydro-2H-benzo[b][1,4]oxazin-8-yl)ethylamino)ethyl)propanamide;
3-(3-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(3,5-difluorophenethoxy)-N-(2-(2-(8-hydroxy-2-oxo-1,2-dihydroquinolin-5-yl)ethylamino)ethyl)propanamide;
(R)—N-sec-Butyl-N-(2-(2-(8-hydroxy-2-oxo-1,2-dihydroquinolin-5-yl)ethylamino)ethyl)-3-phenethoxypropanamide;
(R)—N-(2-(2-(8-Hydroxy-2-oxo-1,2-dihydroquinolin-5-yl)ethylamino)ethyl)-N-(3-methylbutan-2-yl)-3-phenethoxypropanamide;
N-Cycloheptyl-N-(2-(2-(8-hydroxy-2-oxo-1,2-dihydroquinolin-5-yl)ethylamino)ethyl)-3-phenethoxypropanamide;
N-(2-(2-(8-Hydroxy-2-oxo-1,2-dihydroquinolin-5-yl)ethylamino)ethyl)-N-isopropyl-3-phenethoxypropanamide;
(R)—N-(Hexan-2-yl)-N-(2-(2-(8-hydroxy-2-oxo-1,2-dihydroquinolin-5-yl)ethylamino)ethyl)-3-phenethoxypropanamide;
N-(2-(2-(8-Hydroxy-2-oxo-1,2-dihydroquinolin-5-yl)ethylamino)ethyl)-N-neopentyl-3-phenethoxypropanamide;
N-Cyclohexyl-3-(2,4-difluorophenethoxy)-N-(2-(2-(8-hydroxy-2-oxo-1,2-dihydroquinolin-5-yl)ethylamino)ethyl)propanamide;
N-Cyclohexyl-3-(2,3-difluorophenethoxy)-N-(2-(2-(8-hydroxy-2-oxo-1,2-dihydroquinolin-5-yl)ethylamino)ethyl)propanamide;
N-Cyclohexyl-3-(3,4-difluorophenethoxy)-N-(2-(2-(8-hydroxy-2-oxo-1,2-dihydroquinolin-5-yl)ethylamino)ethyl)propanamide;
N-Cyclohexyl-3-(2,6-difluorophenethoxy)-N-(2-(2-(8-hydroxy-2-oxo-1,2-dihydroquinolin-5-yl)ethylamino)ethyl)propanamide;
N-Cyclohexyl-3-(2,5-difluorophenethoxy)-N-(2-(2-(8-hydroxy-2-oxo-1,2-dihydroquinolin-5-yl)ethylamino)ethyl)propanamide;
N-cyclohexyl-3-(2,4-difluorophenethoxy)-N-(2-(2-(5-hydroxy-3-oxo-3,4-dihydro-2H-benzo[b][1,4]oxazin-8-yl)ethylamino)ethyl)propanamide;
N-Cyclohexyl-3-(3,4-difluorophenethoxy)-N-(2-(2-(5-hydroxy-3-oxo-3,4-dihydro-2H-benzo[b][1,4]oxazin-8-yl)ethylamino)ethyl)propanamide;
N-Cyclohexyl-3-(2,3-difluorophenethoxy)-N-(2-(2-(5-hydroxy-3-oxo-3,4-dihydro-2H-benzo[b][1,4]oxazin-8-yl)ethylamino)ethyl)propanamide;
3-(2-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide;
3-(4-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide;
N-Cycloheptyl-N-(2-(2-(5-hydroxy-3-oxo-3,4-dihydro-2H-benzo[b][1,4]oxazin-8-yl)ethylamino)ethyl)-3-phenethoxypropanamide;
N-Cycloheptyl-N-(2-(2-(5-hydroxy-3-oxo-3,4-dihydro-2H-benzo[b][1,4]oxazin-8-yl)ethylamino)ethyl)-3-(2-(pyridin-2-yl)ethoxy)propanamide;
N-(2-(2-(5-Hydroxy-3-oxo-3,4-dihydro-2H-benzo[b][1,4]oxazin-8-yl)ethylamino)ethyl)-N-isopropyl-3-phenethoxypropanamide;
(R)—N-(2-(2-(5-Hydroxy-3-oxo-3,4-dihydro-2H-benzo[b][1,4]oxazin-8-yl)ethylamino)ethyl)-N-(3-methylbutan-2-yl)-3-phenethoxypropanamide;
N-Cyclopentyl-N-(2-(2-(5-hydroxy-3-oxo-3,4-dihydro-2H-benzo[b][1,4]oxazin-8-yl)ethylamino)ethyl)-3-phenethoxypropanamide;
N-(2-(2-(5-Hydroxy-3-oxo-3,4-dihydro-2H-benzo[b][1,4]oxazin-8-yl)ethylamino)ethyl)-N-neopentyl-3-phenethoxypropanamide;
3-(3-Chlorophenethoxy)-N-(2-(diethylamino)ethyl)-N-(2-(2-(5-hydroxy-3-oxo-3,4-dihydro-2H-benzo[b][1,4]oxazin-8-yl)ethylamino)ethyl)propanamide; or,
3-(3-chlorophenethoxy)-N-(2-(diethylamino)ethyl)-N-(2-(2-(8-hydroxy-2-oxo-1,2-dihydroquinolin-5-yl)ethylamino)ethyl)propanamide;
   or a pharmaceutically acceptable salt thereof.

The present invention further provides a process for the preparation of a compound of formula (I) or a pharmaceutically acceptable salt thereof as defined above which comprises,

(a) reacting a compound of formula (II)

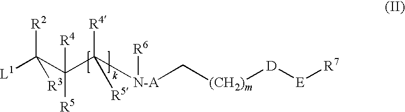

wherein L¹ represents a leaving group (e.g. chlorine, bromine, iodine, methanesulfonate or para-toluenesulfonate) and k, R², R³, R⁴, R⁵, R^{4′}, R^{5′}, R⁶, R⁷, A, D, E and m are as defined in formula (I), with a compound of formula (III) or a suitable salt thereof (e.g. hydrobromide or hydrochloride salt)

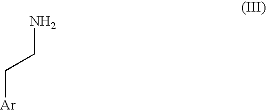

wherein Ar is as defined in formula (I), in the presence of a base (e.g. potassium carbonate, triethylamine or diisopropylethylamine); or
(b) when R² and R³ each represent hydrogen, reacting a compound of formula (IV)

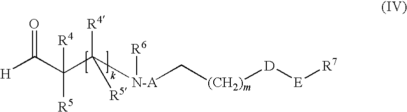

wherein k, R⁴, R⁵, R^{4′}, R^{5′}, R⁶, R⁷, A, D, m and E are as defined in formula (I), with a compound of formula (III) or a suitable salt thereof as defined in (a) above in the presence of a suitable reducing agent (e.g. sodium cyanoborohydride, sodium triacetoxyborohydride, or hydrogen in the presence of a palladium on carbon or palladium oxide catalyst); or
(c) when R² and R³ each represent hydrogen, contacting a compound of formula (V)

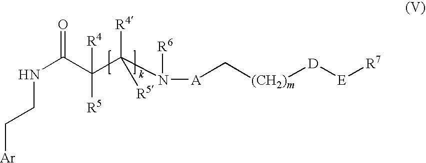

wherein k, Ar, R⁴, R⁵, R^{4′}, R^{5′}, R⁶, R⁷, A, D, m and E are as defined in formula (I) with a suitable reducing agent (e.g. lithium aluminium hydride or borane tetrahydrofuran complex);
and optionally after (a), (b) or (c) carrying out one or more of the following:
a) converting the compound obtained to a further compound of the invention
b) forming a pharmaceutically acceptable salt of the compound.

In process (a), the reaction may conveniently be carried out in an organic solvent such as N,N-dimethylformamide, ethanol, n-butanol or dimethyl sulfoxide, at a temperature, for example, in the range from 50 to 140° C.

In process (b), the reaction may conveniently be carried out in an organic solvent such as methanol, ethanol, dichloromethane, acetic acid, N-Methyl-2-pyrrolidinone or N,N-dimethylformamide containing up to 10% w of water and acetic acid.

In process (c), the reaction may conveniently be carried out in an organic solvent such as tetrahydrofuran, at a temperature, for example, in the range from 0 to 60° C.

Compounds of formula (II) in which A represents carbonyl may be prepared by reacting a compound of formula (X)

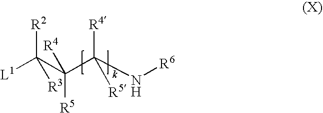

wherein L¹, k, R², R³, R⁴, R⁵, R^{4′}, and R^{5′} R⁶ are as defined in formula (II), with a compound of formula (XI)

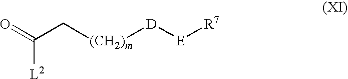

wherein L² represents a leaving group (such as hydroxyl or halogen, e.g. chlorine) and m, E, D and R⁷ are as defined in formula (II).

When L² represents hydroxyl, the reaction is conveniently carried out in the presence of an activating reagent, for example, carbonyldiimidazole, 2,4,6-tripropyl-1,3,5-trioxa-2,4,6-triphosorinan-2,4,6-trioxide or O-(7-azabenzotriazol-1-yl)-N,N,N′,N′-tetramethyluroniumhexafluorophosphate (HATU), in an organic solvent, for example, N,N-dimethylformamide or dichloromethane, at a temperature, for example in the range from 0 to 60° C., with a suitable base, if required, for example triethylamine or diisopropylethylanmine.

When L² represents chlorine, the reaction is conveniently carried out in the presence of a base, for example, triethylamine or diisopropylethylamine in an organic solvent, for example, dichloromethane or tetrahydrofuran at a temperature, for example, in the range from 0 to 25° C. Alternatively, when L² represents chlorine, the reaction may be carried out under bi-phasic conditions, using an organic solvent which is immiscible with water, such as dichloromethane, in the presence of an aqueous solution of a base, for example sodium hydrogen carbonate.

Compounds of formula (II) in which A represents methylene may be prepared by contacting a corresponding compound of formula (II) in which A represents carbonyl with a reducing agent, for example, lithium aluminium hydride or borane tetrahydrofuran complex in an organic solvent, for example, tetrahydrofuran at a temperature, for example in the range from 0 to 60° C.

Compounds of formula (II) in which A represents sulphonyl may be prepared by reacting a compound of formula (X) as defined above with a compound of formula (XII)

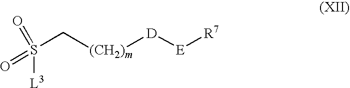

wherein L³ represents a leaving group (e.g. halogen) and m, E, D and R⁷ are as defined in is formula (II). The reaction may be carried out in the presence of a base, for example, triethylamine or diisopropylethylamine in an organic solvent, for example, dichloromethane or tetrahydrofuran at a temperature, for example, in the range from 0 to 25° C.

Compounds of formula (III) may be prepared by processes which are described in the literature and which will be familiar to those skilled in the art, for example J. Med. Chem. 1985, 28, 1803. Alternatively, compounds of formula (III) can be prepared by using or adapting the methods described in the Examples below.

Compounds of formula (IV) may be prepared by treating a compound of formula (XIII)

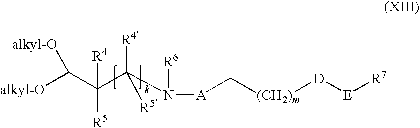

in which k, R⁴, R⁵, R^{4′}, R^{5′}, R⁶, R⁷, A, D, m and E are as defined in formula (IV), with a strong acid such as concentrated hydrochloric acid or para-toluenesulphonic acid in an organic solvent such as 1,4-dioxane, acetone or dichloromethane at a temperature, for example, of 25° C.

Compounds of formula (IV) may alternatively be prepared by oxidising a compound of formula (XIV)

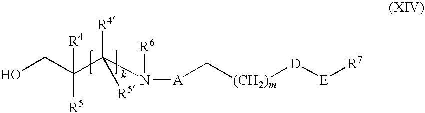

wherein k, R⁴, R⁵, R^{4′}, R^{5′}, R⁶, R⁷, A, D, m and E are as defined in formula (IV), with an oxidising agent, for example pyridinium chloro chromate or Dess-Martin periodinane in an organic solvent, for example, dichloromethane at a temperature, for example, of 25° C. Other oxidative procedures may also be employed as known to persons skilled in the art, for example, the Swern oxidation which is outlined in Synthesis, 1981, 3, 165.

Compounds of formula (V) may be prepared by reacting a compound of formula (XV)

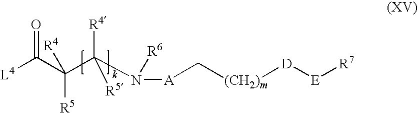

wherein L⁴ represents a leaving group (e.g. chlorine or hydroxyl) and k, R⁴, R⁵, R^{4′}, R^{5′}, R⁶, R⁷, A, D, m and E are as defined in formula (V), with a compound of formula (III) or a suitable salt thereof as defined above.

When L⁴ represents chlorine, the reaction is conveniently carried out in the presence of a base, for example, triethylamine or diisopropylethylamine in an organic solvent, for example, dichloromethane at a temperature, for example, in the range from 0 to 25° C. Alternatively, when L⁴ represents chlorine, the reaction may be carried out under bi-phasic conditions, using an organic solvent which is immiscible with water, such as dichloromethane, in the presence of an aqueous solution of a base, for example sodium hydrogen carbonate.

When L⁴ represents hydroxyl, the reaction is conveniently carried out in the presence of an activating reagent, for example, carbonyldiimidazole, 2,4,6-tripropyl-1,3,5-trioxa-2,4,6-triphosorinan-2,4,6-trioxide or O-(7-azabenzotriazol-1-yl)-N,N,N′,N′-tetramethyluroniumhexafluorophosphate (HATU), in an organic solvent, for example, N,N-dimethylformamide or dichloromethane, at a temperature, for example in the range from 0 to 60° C., with a suitable base, if required, for example triethylamine or diisopropylethylamine.

Compounds of formula (XIII) in which A represents carbonyl may be prepared by reacting a compound of formula (XVI)

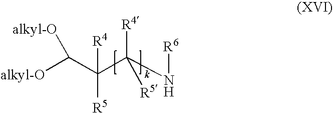

wherein k, R⁴, R⁵, R^{4′}, R^{5′} and R⁶ are as defined in formula (XIII), with a compound of formula (XI) as defined above.

Compounds of formula (XIII) in which A represents sulphonyl may be prepared by reacting a compound of formula (XVI) as defined above with a compound of formula (XII) as defined above, e.g. in the presence of a base such as triethylamine or diisopropylethylamine in an organic solvent such as dichloromethane or tetrahydrofuran at a temperature, for example, in the range from 0 to 25° C.

Compounds of formula (XIII) in which A represents methylene may be prepared by reacting a compound of formula (XVI) as defined above with a compound of formula (XVII)

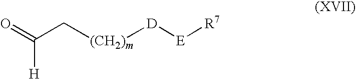

wherein m, E, D and R⁷ are as defined in formula (XIII), in the presence of a reducing agent, for example, sodium cyanoborohydride or sodium triacetoxyborohydride in an organic solvent, for example, methanol, ethanol, dichloromethane or N,N-dimethylformamide containing, for example, 0-10% w water. The reaction could also be performed in an organic solvent, for example, ethanol, acetic acid or methanol (or a combination of either) under an atmosphere of hydrogen gas with a suitable catalyst, for example, 5-10% w palladium on carbon or platinum oxide.

Compounds of formulae (XIV) and (XV) may be prepared by processes similar to those described for the preparation of compounds of formula (XIII).

Compounds of formula (XVI) may be prepared by reacting a compound of formula (XVIII)

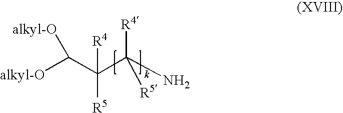

wherein k, R⁴, R⁵, R^{4′} and R^{5′} are as defined in formula (XVI), with a compound of formula (XIX), R⁶—CHO, wherein R⁶ is as defined in formula (XVI), in the presence of a reducing agent, for example, sodium cyanoborohydride or sodium triacetoxyborohydride in an organic solvent, for example, methanol, ethanol, dichloromethane or N,N-dimethylformamide containing, for example, 0-10% w water. The reaction could also be performed in an organic solvent, for example, ethanol, acetic acid or methanol (or a combination of either) under an atmosphere of hydrogen gas with a suitable catalyst, for example, 5-10% w palladium on carbon or platinum oxide.

Compounds of formula (I) can be converted into further compounds of formula (I) using standard procedures.

For example, compounds of formula (I) in which R¹⁰ represents a 3- to 12-membered ring system (e.g. piperidinyl) substituted by a C₁₋₆ alkoxycarbonyl substituent group may be converted to the corresponding compounds in which the ring system is unsubstituted by treating the former with, for example, trifluoroacetic acid or anhydrous hydrogen chloride, in an organic solvent such as dichloromethane or 1,4-dioxane at a temperature, for example, in the range from 15 to 30° C.

The invention further provides a process for the preparation of a compound of formula (XX) (as a salt):

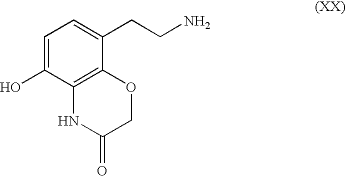

the process comprising reducing a compound of formula (XXI):

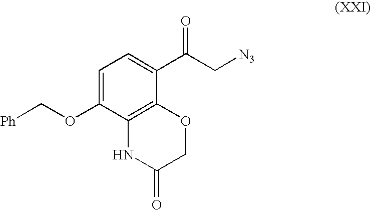

with hydrogen in the presence of a suitable palladium catalyst (such as palladium on carbon) in a aqueous medium and in the presence of a suitable strong acid (such as hydrochloric acid. The process is advantageous because it allows the compound of formula (XX) to be prepared by reduction of three groups (azide, ketone and deprotection) of the compound of formula (XXI) in one-pot. The preparation of a compound of formula (XXI) is presented in Route A.

Routes B, C, D and E all show the preparation of compounds of formula (I) wherein R², R³, R⁴ and R⁵ are all hydrogen, k is 0, m is 1, A is C(O), and E is CH₂CH₂. The routes can be adapted using literature methods to obtain compounds of formula (I) wherein these variables are other than the values just recited. In Routes B and C the variable D is NH, in Route D the variable D is NH or O, while in Route E the variable D is O. In these Routes NMP is N-methyl-2-pyrrolidinone, PG is a Protecting Group, and LG is a Leaving Group.

Route D begins with the preparation of an amide compound:

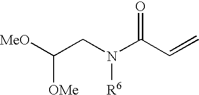

and this compound utilises an alkenyl group both as a protecting group and as a synthon later in the process. There is no literature precedent for such an intermediate and so in another aspect the present invention provides an intermediate of formula (XXII):

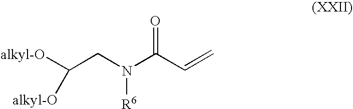

wherein R⁶ is as defined above; and alkyl is, for example, C₁₋₁₀ alkyl.

It can be seen that the process at the beginning of Route D can be adapted in the following way:

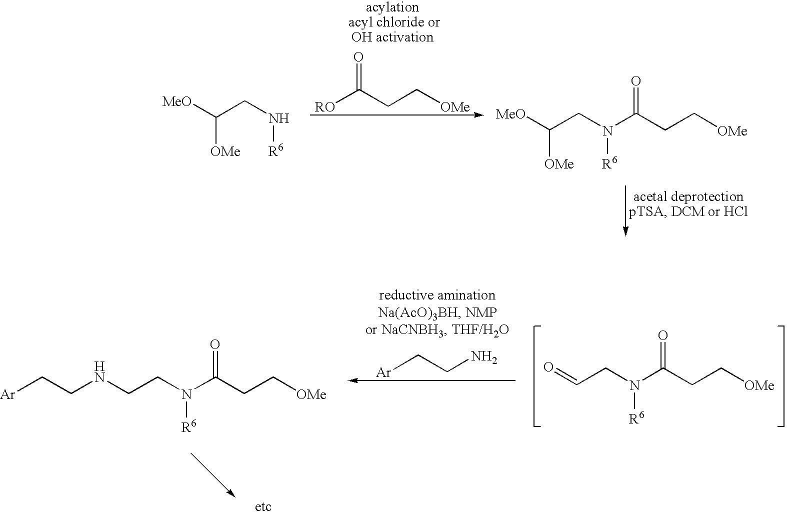

and so in a further aspect the present invention provides a compound of formula (XXIII):

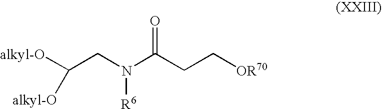

wherein R⁶ is as defined above; R⁷⁰ is C₁₋₆ alkyl; and alkyl is, for example, C₁₋₁₀ alkyl.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

The compounds of formula (I) above may be converted to a pharmaceutically acceptable salt thereof, for example as described above, such as an acid addition salt which is a hydrochloride, hydrobromide, trifluoroacetate, sulphate, phosphate, acetate, fumarate, maleate, tartrate, lactate, citrate, pyruvate, succinate, oxalate, methanesulphonate or p-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired. When in solid crystalline form a compound of formula (I) can be in the form of a co-crystal with another chemical entity and the invention encompasses all such co-crystals.

The compounds of formula (I) and their pharmaceutically acceptable salts can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including anlylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as to osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodeimatitis; seborrhoeic is dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis (including ulcerative colitis, microscopic colitis and indeterminant colitis) proctitis, pruritis ani, coeliac disease, irritable bowel disorder, irritable bowel syndrome, non-inflammatory diarrhoea, and food-related allergies which may have effects remote from the gut (for example migraine, rhinitis or eczema);
7. abdominal: hepatitis, including autoimnuune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzeimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins; and,
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention still further provides a method of treating, or reducing the risk of, an inflammatory disease or condition (including a reversible obstructive airways disease or condition) which comprises administering to a patient in need thereof a therapeutically effective amount of a compound of formula (I) or a pharmaceutically acceptable salt thereof as hereinbefore defined.

In particular, the compounds of this invention may be used in the treatment of adult respiratory distress syndrome (ARDS), pulmonary emphysema, bronchitis, bronchiectasis, chronic obstructive pulmonary disease (COPD), asthma and rhinitis.

For the above-mentioned therapeutic uses the dosage administered will, of course, vary with the compound employed, the mode of administration, the treatment desired and the disorder indicated. For example, the daily dosage of the compound of the invention, if inhaled, may be in the range from 0.05 micrograms per kilogram body weight (μg/kg) to 100 micrograms per kilogram body weight (μg/kg). Alternatively, if the compound is administered orally, then the daily dosage of the compound of the invention may be in the range from 0.01 micrograms per kilogram body weight (μg/kg) to 100 milligrams per kilogram body weight (mg/kg).

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

The invention therefore further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with the following agents: non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid receptor agonist, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof. A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as β2×7; or (xxvii) inhibitor of transcription factor activation such as NFkB, API, or STATS.

In a further aspect the present invention provides a combination (for example for the treatment of COPD, asthma or allergic rhinitis) of a compound of formula (I) and one or more agents selected from the list comprising:
a non-steroidal glucocorticoid receptor (GR-receptor) agonist;
a PDE4 inhibitor including an inhibitor of the isoform PDE4D;
a muscarinic receptor antagonist (for example a M1, M2 or M3 antagonist, such as a selective M3 antagonist) such as ipratropium bromide, tiotropium bromide, oxitropium bromide, pirenzepine or telenzepine;
a modulator of chemokine receptor function (such as a CCR1 receptor antagonist);
a steroid (such as budesonide); or,
an inhibitor of a kinase function (for example IKK2 or p38).

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The present invention will now be further explained by reference to the following illustrative examples.

### General Methods

¹H NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references.

Mass spectra were recorded on a Agilant MSD (+ve and −ve APCI and EI) or a Waters ZMD (+ve and −ve EI) following analytical HPLC on an Agilant 1100.

Flash chromatography was carried out on silica causing Biotage FLASH™ or equivalent, for example Biotage Flashmaster™ or Isolute columns. Unless stated otherwise, starting materials were commercially available. All solvents and commercial reagents were of laboratory grade and were used as received.

Preparative HPLC was carried out using either a Phenomenex Gemini C18 5 μm, a Waters Xterra C8 5 μm or a Waters Xbridge C8 5 μm using aceonitrile in either aqueous ammonia or aqeuous trifluoroacetic acid; or a Waters Sunfire C18 5 μm using acetonitrile in aqueous trifluoroacetic acid.

Purification by reversed phase preparative HPLC. Method A is using an Xterra® C8 5 micron 19×50 mm column eluting with a gradient of acetonitrile in 0.2% aqueous 0.880 ammonia over 6 min. at 20 ml/min. Method B is using an Atlantis™ C18 5 micron 19×50 mm column eluting with a gradient of acetonitrile in 0.1% aqueous TFA.

The abbreviations or terms used in the examples have the following meanings:

SCX: Solid phase extraction with a sulfonic acid sorbent
HPLC: High performance liquid chromatography

### DMF: N,N-Dimethylformamide

NMP: N-Methyl-2-pyrrolidinone
T3P: 2,4,6-tripropyl-1,3,5-trioxa-2,4,6-triphosorinan-2,4,6-trioxide

### EXAMPLE 1

### N-Butyl-N-(2-(2-(8-hydroxy-2-oxo-1,2-dihydroquinolin-5-yl)ethylamino)ethyl)-3-phenethoxypropanamide hydrochloride

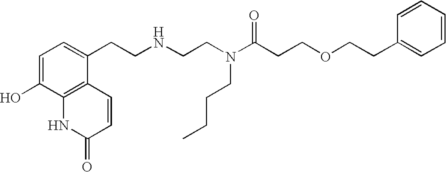

### Step i) N-Butyl-N-(2-hydroxyethyl)-3-phenethoxypropanamide

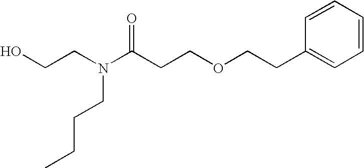

To a stirred solution of tert-butyl 3-phenethoxypropanoate (2.5 g; EP0411409) in dichloromethane (10 mL) was added trifluoroacetic acid (5 mL) and the mixture was stirred for 4 h and then concentrated. The residue was dissolved in dichloromethane (10 mL) and oxalyl chloride (3 mL) added. The reaction was stirred for 2 h and concentrated. The residue was dissolved in dichloromethane (20 mL) and added to a solution of N,N-diisopropylethylamine (3.23 g) and 2-butylaminoethanol (1.41 g) in dichloromethane (20 mL) at 5° C. The reaction was stirred for 1 h and poured into 2N hydrochloric acid (30 mL) and extracted into ethyl acetate (2×30 mL). The organic phase was dried over anhydrous magnesium sulfate, filtered and concentrated to give the sub-titled compound as an oil (1.95 g).

¹H NMR (400 MHz, CDCl₃) δ 7.30-7.18 (m, 5H), 3.83-3.74 (m, 3H), 3.68 (t, 3H), 3.53-3.45 (m, 3H), 3.35-3.25 (m, 2H), 2.89-2.85 (m, 2H), 2.67-2.60 (m, 2H), 1.58-1.47 (m, 2H), 1.36-1.27 (m, 2H), 0.96-0.91 (m, 3H).

### Step ii) N-Butyl-N-(2-oxoethyl)-3-phenethoxypropanamide

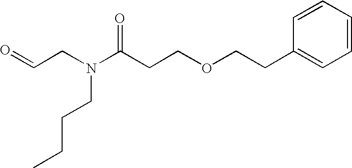

A stirred solution of N-butyl-N-(2-hydroxyethyl)-3-phenethoxypropanamide (1.5 g; Step i) in dichloromethane (60 mL) was treated with Dess-Martin periodinane (2.818 g) and stirring continued for 2 h. The mixture was diluted with dichloromethane, washed with aqueous sodium hydrogen carbonate solution followed by water, dried over magnesium sulfate, filtered and concentrated to give the sub-titled compound as an oil.

### Step iii) N-Butyl-N-(2-(2-(8-hydroxy-2-oxo-1,2-dihydroquinolin-5-yl)ethylamino)ethyl)-3-phenethoxypropanamide hydrochloride

A solution of 5-(2-aminoethyl)-8-hydroxyquinolin-2(1H)-one (0.070 g; J. Med. Chem. 1985, 28, 1803), N-butyl-N-(2-oxoethyl)-3-phenethoxypropanamide (0.079 g) [Step ii], acetic acid (0.014 mL) and methanol (10 mL) was stirred for 1 h, treated with sodium cyanoborohydride (0.019 g) and stirred overnight. The mixture was treated with a few drops of 5 M methanolic ammonia solution and evaporated. The residue was taken up in a little methanol and applied to a 10 g SCX cartridge and washed with methanol. The product was eluted with methanolic ammonia solution and evaporated. The residue was purified by reverse phase preparative HPLC (5 to 50% acetonitrile in 0.2% aqueous ammonia), followed by further preparative HPLC (acetonitrile in 0.1% aqueous trifluoroacetic acid) and product containing fractions concentrated in vacuo to give a gum. The gum was taken up in a little methanol, treated with concentrated hydrochloric acid (1 drop) and evaporated. The hydrochloric acid/methanol treatment was repeated again to afford the titled compound as a sticky gum (14 mg).

MS (APCI+) 480 [M+H]⁺

¹H NMR (300 MHz, DMSO) δ 10.47 (s, 1H), 10.34 (s, 1H), 8.99-8.77 (m, 2H), 8.15-8.10 (m, 1H), 7.31-7.19 (m, 5H), 6.96-6.89 (m, 2H), 6.59-6.56 (m, 1H), 3.69-3.53 (m, 8H), 3.30-3.23 (m, 2H), 3.17-3.04 (m, 4H), 2.80 (t, J=7.0 Hz, 2H), 2.58 (t, J=6.2 Hz, 2H), 1.53-1.23 (m, 4H), 0.92 (t, J=7.2 Hz, 3H)

### EXAMPLE 2

### N-Cyclohexyl-N-(2-(2-(8-hydroxy-2-oxo-1,2-dihydroquinolin-5-yl)ethylamino)ethyl)-3-phenethoxypropanamide

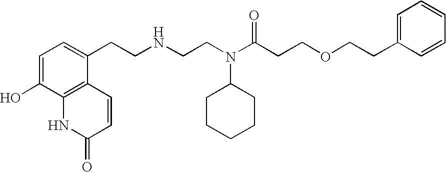

### Step i) N-(2,2-Dimethoxyethyl)cyclohexanamine

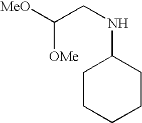

To a solution of cyclohexylamine (4.34 g, 5 mL) in methanol (10 mL) was added glyoxal 1,1-dimethyl acetal (7.58 g, 6.57 mL). The reaction was stirred at ambient temperature for 18 h. After this time 5% palladium on charcoal (Johnson Mattey 38H paste; 0.5 g) was added to the mixture and the reaction hydrogenated under 4 atm pressure of H₂ for 24 h. The reaction mixture was filtered and the filtrates concentrated in vacuo to give the is subtitled compound as a pale-coloured oil (7.85 g).

¹H NMR (299.946 MHz, CDCl₃) δ 4.46 (t, J=5.6 Hz, 1H), 3.38 (s, 6H), 2.75 (d, J=5.6 Hz, 2H), 2.40 (tt, J=10.4, 3.7 Hz, 1H), 1.91-1.83 (m, 2H), 1.76-1.69 (m, 2H), 1.65-1.58 (m, 1H), 1.38-1.00 (m, 6H)

### Step ii) N-Cyclohexyl-N-(2,2-dimethoxyethyl)-3-phenethoxypropanamide

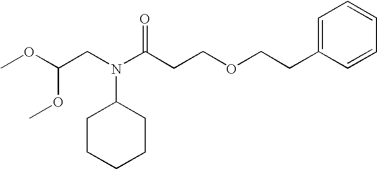

A solution of T3P in tetrahydrofuran (1.57 M; 10.82 mL) was added to a stirred solution of 3-phenethoxypropanoic acid (1.650 g) [Tetrahedron 1998, 54, 12151-60] and N-(2,2-dimethoxyethyl)cyclohexanamine (1.640 mL) [Step i] in acetonitrile (35 mL) at 25° C. The resulting mixture was stirred at 25° C. for 6 h then basified with saturated sodium hydrogen carbonate and extracted into ethyl acetate (2×100 mL). The organic phase was dried over anhydrous magnesium sulfate, filtered and concentrated in vacuo. The crude product was purified by flash chromatography on silica (eluting with 0 to 30% ethyl acetate in isohexane). Product containing fractions were evaporated to dryness to afford the subtitled compound as a yellow oil (2.160 g).

¹H NMR (299.946 MHz, CDCl₃) δ 7.36-7.13 (m, 5H), 4.68-4.32 (m, 1H), 3.86-3.50 (m, 5H), 3.45-3.35 (m, 7H), 3.34-3.28 (m, 1H), 2.95-2.82 (m, 2H), 2.76-2.63 (m, 2H), 1.90-0.94 (m, 10H).

### Step iii) N-Cyclohexyl-N-(2-(2-(8-hydroxy-2-oxo-1,2-dihydroquinolin-5-yl)ethylamino)ethyl)-3-phenethoxypropanamide

A mixture of N-cyclohexyl-N-(2,2-dimethoxyethyl)-3-phenethoxypropanamide (150 mg) [Step ii], p-toluenesulfonic acid monohydrate (162 mg) and dichloromethane (2 mL) was stirred at 25° C. for 2 h. DIPEA (0.4 mL), 5-(2-aminoethyl)-8-hydroxyquinolin-2(1H)-one hydrochloride (120 mg) [J. Med. Chem. 1985, 28, 1803], NMP (2.0 mL) and sodium triacetoxyborohydride (87 mg) were added and the resulting mixture was stirred at 25° C. for 2 h. Saturated aqueous sodium hydrogen carbonate was added and the mixture extrated with dichloromethane. The organic phase was dried over anhydrous magnesium sulfate, filtered and concentrated in vacuo. The crude product was purified by reverse phase preparative HPLC (eluting with a gradient of acetonitrile in 0.2% aqueous trifluoroacetic acid), followed by further preparative HPLC (eluting with a gradient of acetonitrile in aqueous 0.2% ammonia). Product containing fractions were evaporated to dryness to afford the title compound as a yellowish solid (37.0 mg).

MS (APCI+) 506 [M+H]⁺

¹H NMR (399.826 MHz, DMSO) δ 8.04 (dd, J=9.7, 5.6 Hz, 1H), 7.30-7.12 (m, 5H), 6.90-6.82 (m, 2H), 6.50 (d, J=9.7 Hz, 1H), 4.11-3.98 (m, 1H), 3.65-3.51 (m, 4H), 3.21-3.13 (m, 2H), 2.92-2.83 (m, 2H), 2.77 (q, J=6.8 Hz, 214), 2.73-2.65 (m, 2H), 2.63-2.48 (m, 4H), 1.77-0.97 (m, 10H).

### EXAMPLE 3

### 3-(3-Chlorophenethylamino)-N-cycloheptyl-N-(2-(2-(8-hydroxy-2-oxo-1,2-dihydroquinolin-5-yl)ethylamino)ethyl)propanamide bis(trifluoroacetate)

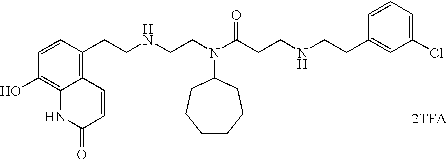

### Step i) Benzyl 2,2-dimethoxyethyl(2-(8-hydroxy-2-oxo-1,2-dihydroquinolin-5-yl)ethyl)carbamate

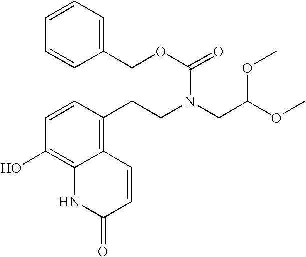

Triethylamine (2.027 mL) was added to a mixture of 5-(2-aminoethyl)-8-hydroxyquinolin-2(1H)-one hydrochloride (3.5 g) [J. Med. Chem. 1985, 28, 1803] in tetrahydrofuran (35 mL) and water (3.5 mL) and the reaction was cooled to 15° C. Glyoxal 1,1-dimethyl acetal (2.172 mL) and NMP (10 mL) were added, followed after 5 minutes by portionwise addition of sodium cyanoborohydride (1.828 g), maintaining the temperature below 15° C. The mixture was warmed to ambient temperature and stirred under nitrogen for 30 minutes. Water (30 mL) was added, followed by the sodium hydrogen carbonate (1.209 g). The resulting solution was cooled to −5° C. and stirred for 30 minutes before the addition of benzyl chloroformate (1.038 mL). After 15 minutes at −5° C., the reaction was diluted with ethyl acetate and the phases separated. The aqueous phase was extracted with further ethyl acetate (twice) and the combined organics were washed with 0.2 M hydrochloric acid, water (twice) and brine, then dried over anhydrous sodium sulfate, filtered and concentrated in vacuo to afford the subtitled compound as a yellow gum (5 g).

MS (ES+) 427 [M+H]⁺

### Step ii) Benzyl 2-(8-hydroxy-2-oxo-1,2-dihydroquinolin-5-yl)ethyl(2-oxoethyl)carbamate

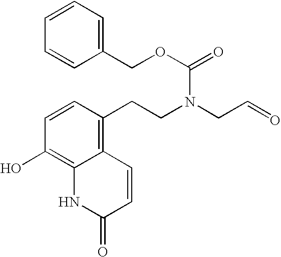

Benzyl 2,2-dimethoxyethyl(2-(8-hydroxy-2-oxo-1,2-dihydroquinolin-5-yl)ethyl)carbamate (5 g) [Step i] was dissolved in acetone (50 mL) and 2 M hydrochloric acid (30 mL) was added. The mixture was stirred overnight and then partially concentrated in vacuo. The remaining aqueous solution was extracted with ethyl acetate (×3) and the combined organics were washed with brine, dried over anhydrous sodium sulfate, filtered and concentrated in vacuo to afford the subtitled compound as a pale yellow solid (4.33 g).

MS (ES+) 381 [M+H]⁺.

### Step iii) Benzyl 2-(cycloheptylamino)ethyl(2-(8-hydroxy-2-oxo-1,2-dihydroquinolin-5-yl)ethyl)carbamate

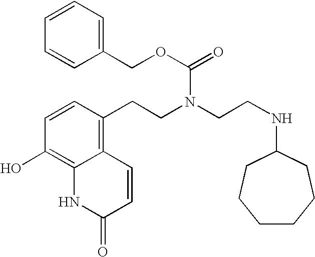

Cycloheptylamine (2.116 mL, 16.61 mmol) was added to a solution of benzyl 2-(8-hydroxy-2-oxo-1,2-dihydroquinolin-5-yl)ethyl(2-oxoethyl)carbamate (3.16 g) [Step ii] in tetrahydrofuran (30 mL). The reaction was stirred under nitrogen for 15 minutes, then cooled to ˜10° C. and sodium triacetoxyborohydride (3.52 g) was added. The reaction was allowed to return to ambient temperature and stirred for 2 h. Saturated aqueous sodium hydrogen carbonate solution was added and the mixture extracted three time with ethyl acetate. The combined organics were washed with water and brine, dried over anhydrous sodium sulfate, filtered and concentrated in vacuo. This crude product was purified by flash chromatography on silica (eluting with 5-10% methanol in dichloromethane). Product containing fractions were concentrated in vacuo to afford the subtitled compound as a yellow gum (1.236 g).

MS (ES+) 478 [M+H]⁺

### Step iv) Benzyl 2-(N-cycloheptylacrylamido)ethyl(2-(8-hydroxy-2-oxo-1,2-dihydroquinolin-5-yl)ethyl)carbamate

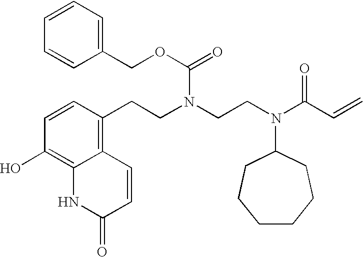

To a stirred solution of benzyl 2-(cycloheptylamino)ethyl(2-(8-hydroxy-2-oxo-1,2-dihydroquinolin-5-yl)ethyl)carbamate (1.23 g) [Step iii] in dichloromethane (30 mL) was added triethylamine (1.436 mL) followed by acryloyl chloride (0.628 mL) and the mixture was stirred at ambient temperature for 18 h. Methanol (20 mL) was added, followed by potassium carbonate (1 g) and the mixture was stirred for a further 18 h. Water was added and the pH was adjusted to 7 with 2 M hydrochloric acid till pH 7. The resulting mixture was extracted three times with dichloromethane and the combined organics were washed with brine, dried over anhydrous sodium sulfate, filtered and concentrated in vacuo to afford the subtitled compound as a yellow solid (1.54 g).

MS (ES+ 532) [M+H]⁺

### Step v) 3-(3-Chlorophenethylamino)-N-cycloheptyl-N-(2-(2-(8-hydroxy-2-oxo-1,2-dihydroquinolin-5-yl)ethylamino)ethyl)propanamide bis(trifluoroacetate)

Benzyl 2-(N-cycloheptylacrylamido)ethyl(2-(8-hydroxy-2-oxo-1,2-dihydroquinolin-5-yl)ethyl)carbamate (100 mg) [Step iv] and 2-(3-chlorophenyl)ethylamine (88 mg) were combined in ethanol (1.1 mL) and heated within a CEM Discover microwave at 100° C. until the reaction was completed, as judged by LCMS (30 minutes). The mixture was then concentrated in vacuo prior to addition of acetic acid (3 mL) followed by 33% HBr in acetic acid (2 mL). The resulting solution was stirred for 2 h then concentrated in vacuo and the residue purified by reverse phase preparative HPLC (eluting with acetonitrile 0.1% aqueous trifluoroacetic acid). Product containing fractions were combined and concentrated in vacuo to afford the title compound as a solid (50 mg).

MS (APCI+) 553 [M+H]⁺

¹H NMR (399.826 MHz, DMSO) δ 10.47 (s, 1H), 10.34 (s, 1H), 8.62-8.46 (m, 4H), 8.08-8.01 (m, 1H), 7.41-7.31 (m, 3H), 7.27-7.22 (m, 1H), 6.94-6.87 (m, 2H), 6.58 (d, J=10.0 Hz, 1H), 3.72-3.63 (m, 1H), 3.49-3.43 (m, 2H), 3.30-3.01 (m, 10H), 2.99-2.92 (m, 2H), 2.85-2.79 (m, 2H), 1.80-1.37 (m, 12H).

### EXAMPLE 4

### 3-(4-Chlorophenethylamino)-N-cycloheptyl-N-(2-(2-(8-hydroxy-2-oxo-1,2-dihydroquinolin-5-yl)ethylamino)ethyl)propanamide bis(trifluoroacetate)

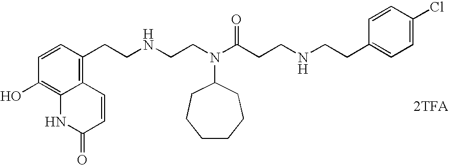

Prepared by an analogous procedure to Example 3, Step v, using 2-(4-chlorophenyl)ethylamine (88 mg) in place of 2-(3-chlorophenyl)ethylamine.

MS (APCI+) 553 [M+H]⁺

¹H NMR (399.826 MHz, DMSO) δ 10.47 (s, 1H), 10.35 (s, 1H), 8.66-8.44 (m, 4H), 8.08-8.01 (m, 1H), 7.43-7.38 (m, 2H), 7.33-7.27 (m, 2H), 6.93-6.87 (m, 2H), 6.58 (d, J=10.0 Hz, 1H), 3.71-3.62 (m, 1H), 3.50-3.42 (m, 2H), 3.27-3.01 (m, 10H), 2.96-2.90 (m, 2H), 2.84-2.78 (m, 2H), 1.80-1.39 (m, 12H).

### EXAMPLE 5

### N-Cycloheptyl-3-(3-fluorophenethylamino)-N-(2-(2-(8-hydroxy-2-oxo-1,2-dihydroquinolin-5-yl)ethylamino)ethyl)propanamide bis(trifluoroacetate)

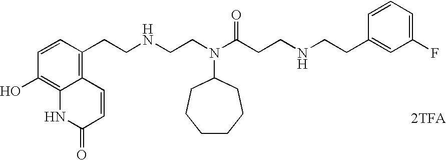

Prepared by an analogous procedure to Example 3, Step v, using 2-(3-fluorophenyl)ethylamine (79 mg) in place of 2-(3-chlorophenyl)ethylamine.

MS (APCI+) 537 [M+H]⁺

¹H NMR (399.825 MHz, CD₃OD) δ 8.15 (d, J=9.9 Hz, 1H), 7.36-7.30 (m, 1H), 7.11-6.94 (m, 5H), 6.67 (d, J=9.5 Hz, 1H), 3.79-3.70 (m, 1H), 3.58 (t, J=6.0 Hz, 2H), 3.36-3.26 (m, 4H), 3.24-3.16 (m, 6H), 3.06-3.00 (m, 2H), 2.89 (t, J=6.5 Hz, 2H), 1.86-1.45 (m, 12H).

### EXAMPLE 6

### N-Cycloheptyl-3-(4-fluorophenethylamino)-N-(2-(2-(8-hydroxy-2-oxo-1,2-dihydroquinolin-5-yl)ethylamino)ethyl)propanamide bis(trifluoroacetate)

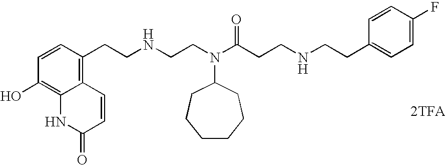

Prepared by an analogous procedure to Example 3, Step v, using 2-(4-fluorophenyl)ethylamine (81 mg) and ethanol (2 mL) in place of 2-(3-chlorophenyl)ethylamine and ethanol (1.1 mL).

MS (APCI+) 537 [M+H]⁺

¹H NMR (399.825 MHz, CD₃OD) δ 8.15 (d, J=9.8 Hz, 1H), 7.32-7.26 (m, 2H), 7.08-6.94 (m, 4H), 6.67 (d, J=9.5 Hz, 1H), 3.79-3.70 (m, 1H), 3.60-3.55 (m, 2H), 3.34-3.26 (m, 4H), 3.25-3.16 (m, 6H), 3.03-2.97 (m, 2H), 2.91-2.87 (m, 2H), 1.87-1.45 (m, 12H).

### EXAMPLE 7

### N-Cycloheptyl-N-(2-(2-(8-hydroxy-2-oxo-1,2-dihydroquinolin-5-yl)ethylamino)ethyl)-3-(phenethylamino)propanamide bis(trifluoroacetate)

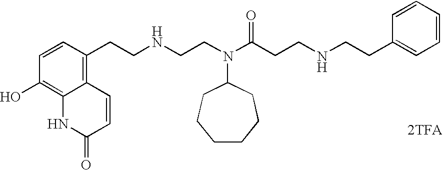

Prepared by an analogous procedure to Example 6 using 2-phenylethylamine (68 mg) in place of 2-(4-fluorophenyl)ethylamine.

MS (APCI+) 519 [M+H]⁺

¹H NMR (399.825 MHz, CD₃OD) δ 8.15 (d, J=9.8 Hz, 1H), 7.34-7.20 (m, 5H), 7.03-6.94 (m, 2H), 6.67 (d, J=9.9 Hz, 1H), 3.79-3.70 (m, 1H), 3.60-3.55 (m, 2H), 3.35-3.26 (m, 4H), 3.25-3.16 (m, 6H), 3.04-2.98 (m, 2H), 2.91-2.86 (m, 2H), 1.87-1.44 (m, 12H).

### EXAMPLE 8

### N-Cycloheptyl-N-(2-(2-(8-hydroxy-2-oxo-1,2-dihydroquinolin-5-yl)ethylamino)ethyl)-3-(3-methylphenethylamino)propanamide bis(trifluoroacetate)

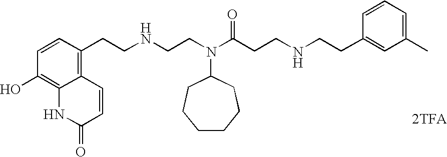

Prepared by an analogous procedure to Example 6 using 2-(3-methylphenyl)ethylamine (76 mg) in place of 2-(4-fluorophenyl)ethylamine.

MS (APCI+) 533 [M+H]⁺

¹H NMR (399.825 MHz, CD₃OD) δ 8.15 (d, J=9.7 Hz, 1H), 7.18 (t, J=7.5 Hz, 1H), 7.10-6.94 (m, 5H), 6.66 (d, J=9.7 Hz, 1H), 3.79-3.69 (m, 1H), 3.60-3.55 (m, 2H), 3.33-3.25 (m, 4H), 3.24-3.16 (m, 6H), 2.99-2.93 (m, 2H), 2.90-2.86 (m, 2H), 2.28 (s, 3H), 1.86-1.44 (m, 12H).

### EXAMPLE 9

### (S)—N-Cycloheptyl-N-(2-(2-(8-hydroxy-2-oxo-1,2-dihydroquinolin-5-yl)ethylamino)ethyl)-3-(2-phenylpropylamino)propanamide bis(trifluoroacetate)

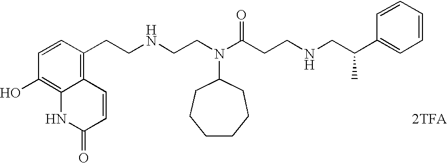

Prepared by an analogous procedure to Example 6 using (S)-2-phenylpropan-1-amine (76 mg) in place of 2-(4-fluorophenyl)ethylamine.

MS (APCI+) 533 [M+H]⁺

¹H NMR (399.825 MHz, CD₃OD) δ 8.14 (d, J=10.2 Hz, 1H), 7.35-7.27 (m, 4H), 7.24-7.19 (m, 1H), 6.99 (dd, J=17.3, 8.3 Hz, 2H), 6.67 (d, J=9.8 Hz, 1H), 3.75-3.65 (m, 1H), 3.57-3.52 (m, 2H), 3.35-3.24 (m, 4H), 3.21-3.12 (m, 7H), 2.87-2.82 (m, 2H), 1.84-1.41 (m, 12H), 1.35 (d, J=6.9 Hz, 3H).

### EXAMPLE 10

### N-Cycloheptyl-3-(3,5-difluorophenethylamino)-N-(2-(2-(8-hydroxy-2-oxo-1,2-dihydroquinolin-5-yl)ethylamino)ethyl)propanamide bis(trifluoroacetate)

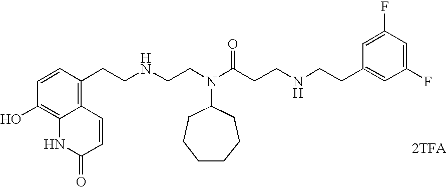

Prepared by an analogous procedure to Example 6 using 2-(3,5-difluorophenyl)ethanamine hydrochloride (109 mg) and N,N-diisopropylethylamine (0.164 mL) in place of 2-(4-fluorophenyl)ethylamine.

MS (APCI+) 555 [M+H]⁺

¹H NMR (399.825 MHz, CD₃OD) δ 8.15 (d, J=9.8 Hz, 1H), 7.03-6.90 (m, 4H), 6.87-6.80 (m, 1H), 6.67 (d, J=10.1 Hz, 1H), 3.79-3.70 (m, 1H), 3.60-3.55 (m, 2H), 3.37-3.28 (m, 4H), 3.24-3.16 (m, 6H), 3.06-3.00 (m, 2H), 2.92-2.87 (m, 2H), 1.87-1.45 (m, 12H).

### EXAMPLE 11

### N-Cycloheptyl-3-(3-ethoxyphenethylamino)-N-(2-(2-(8-hydroxy-2-oxo-1,2-dihydroquinolin-5-yl)ethylamino)ethyl)propanamide bis(trifluoroacetate)

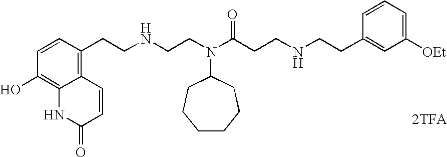

Prepared by an analogous procedure to Example 3, Step v, using 2-(3-ethoxyphenyl)ethanamine (93 mg) and ethanol (3 mL) in place of 2-(3-chlorophenyl)ethylamine and ethanol (1.1 mL).

MS (APCI+) 563 [M+H]⁺

¹H NMR (399.825 MHz, CD₃OD) δ 8.16 (d, J=9.7 Hz, 1H), 7.25-7.15 (m, 1H), 6.98 (d, J=13.8 Hz, 2H), 6.81 (d, J=1.3 Hz, 2H), 6.78-6.74 (m, 1H), 6.66 (d, J=9.7 Hz, 1H), 3.96 (d, J=7.2 Hz, 2H), 3.61-3.55 (m, 3H), 3.35-3.25 (m, 6H), 3.22-3.15 (m, 4H), 3.00-2.94 (m, 2H), 2.91-2.85 (m, 2H), 1.87-1.60 (m, 8H), 1.59-1.48 (m, 4H), 1.32 (t, J=7.0 Hz, 3H).

### EXAMPLE 12

### 3-(2-(Biphenyl-3-yl)ethylamino)-N-cycloheptyl-N-(2-(2-(8-hydroxy-2-oxo-1,2-dihydroquinolin-5-yl)ethylamino)ethyl)propanamide bis(trifluoroacetate)

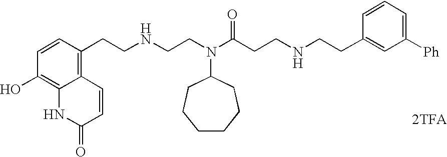

### Step i) Benzyl 2-(3-(3-bromophenethylamino)-N-cycloheptylpropanamido)ethyl(2-(8-hydroxy-2-oxo-1,2-dihydroquinolin-5-yl)ethyl)carbamate bis(trifluoroacetate)

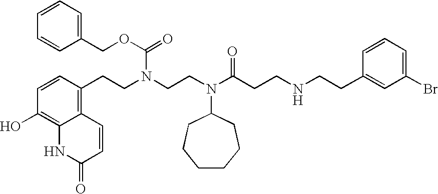

N,N-Diisopropylethylamine (0.111 mL) and 2-(3-bromophenyl)ethanamine (128 mg) were added to a mixture of benzyl 2-(N-cycloheptylacrylamido)ethyl(2-(8-hydroxy-2-oxo-1,2-dihydroquinolin-5-yl)ethyl)carbamate (113 mg) [Example 3, Step iv] in ethanol and the mixture heated within a CEM Discover microwave for 50 minutes at 100° C. The crude material was purified by preparative HPLC (eluting with 5-80% acetonitrile in aqueous 0.1% trifluoroacetic acid). The fractions containing the desired compound were concentrated in vacuo to afford the subtitled compound as an orange solid (40.0 mg).

MS (ES+) 733 [M+H]⁺

### Step ii) 3-(2-(Biphenyl-3-yl)ethylamino)-N-cycloheptyl-N-(2-(2-(8-hydroxy-2-oxo-1,2-dihydroquinolin-5-yl)ethylamino)ethyl)propanamide bis(trifluoroacetate)

Potassium carbonate (30.2 mg), tetrakis(triphenylphosphine)palladium (20 mg), phenylboronic acid (13.33 mg) and benzyl 2-(3-(3-bromophenethylamino)-N-cycloheptylpropanamido)ethyl(2-(8-hydroxy-2-oxo-1,2-dihydroquinolin-5-yl)ethyl)carbamate bis(trifluoroacetate) (40 mg) [Step i] were combined in ethanol and heated within a CEM Discover microwave at 80° C. for 20 minutes. The mixture was concentrated in vacuo and suspended in acetic acid (3 mL). 33% HBr in acetic acid (2 mL) was added and the mixture stirred for 1 h, then concentrated in vacuo. The crude material was purified by reverse phase preparative HPLC (eluting with 5-60% acetonitrile in aqueous 0.1% trifluoroacetic acid). The fractions containing the desired compound were concentrated in vacuo to afford the title compound as a white solid (8.00 mg).

MS (APCI+) 593 [M+H]⁺

¹H NMR (299.947 MHz, CD₃OD) δ 8.18-8.14 (m, 1H), 7.62-7.28 (m, 9H), 7.04-6.97 (m, 2H), 6.70-6.67 (m, 1H), 3.83-3.73 (m, 1H), 3.64-3.36 (m, 12H), 3.17-3.10 (m, 2H), 2.97-2.91 (m, 2H), 1.90-1.52 (m, 12H).

### EXAMPLE 13

### 3-(3-Chlorophenethylamino)-N-cyclohexyl-N-(2-(2-(8-hydroxy-2-oxo-1,2-dihydroquinolin-5-yl)ethylamino)ethyl)propanamide bis(trifluoroacetate)

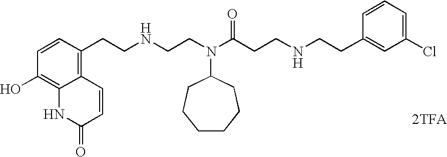

### Step i) Benzyl 2-(cyclohexylamino)ethyl(2-(8-hydroxy-2-oxo-1,2-dihydroquinolin-5-yl)ethyl)carbamate

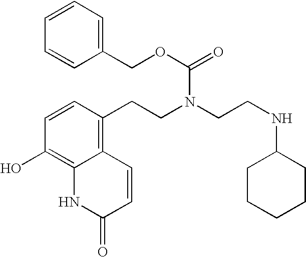

Benzyl 2-(8-hydroxy-2-oxo-1,2-dihydroquinolin-5-yl)ethyl(2-oxoethyl)carbamate (2.6 g) [Example 3, Step ii] was dissolved in water (1 mL) and tetrahydrofuran (10 mL). Cyclohexylamine (1.564 mL) was added and the mixture stirred for 15 minutes. After cooling to ˜10° C., sodium cyanoborohydride (0.859 g) and acetic acid (0.391 mL) were added and the reaction mixture was warmed to ambient temperature and stirred for 1 h. Saturated aqueous sodium hydrogen carbonate was then added and the phases separated. The aqueous phase was extracted with further ethyl actetate and the combined organics were washed with brine, dried over anhydrous sodium sulfate, filtered and concentrated in vacuo. The crude material was purification by flash chromatography silica (eluting with ethyl acetate, then 5-10% methanol in dichloromethane). Product containing fractions were concentrated in vacuo to afford the sub-titled compound as a yellow solid (790 mg)

MS (ES+) 464 [M+H]⁺.

### Step ii) Benzyl 2-(N-cyclohexylacrylamido)ethyl(2-(8-hydroxy-2-oxo-1,2-dihydroquinolin-5-yl)ethyl)carbamate

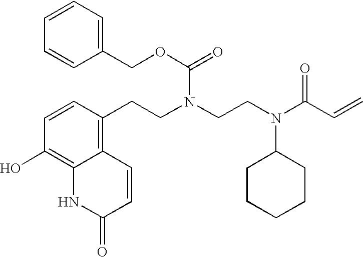

Prepared using the procedure of Example 3, Step iv, using benzyl 2-(cyclohexylamino)ethyl(2-(8-hydroxy-2-oxo-1,2-dihydroquinolin-5-yl)ethyl)carbamate [Step i] in place of benzyl 2-(cycloheptylamino)ethyl(2-(8-hydroxy-2-oxo-1,2-dihydroquinolin-5-yl)ethyl)carbamate.

MS (ES+) 518 [M+H]⁺

### Step iii) 3-(3-Chlorophenethylamino)-N-cyclohexyl-N-(2-(2-(8-hydroxy-2-oxo-1,2-dihydroquinolin-5-yl)ethylamino)ethyl)propanamide bis(trifluoroacetate)

Benzyl 2-(N-cyclohexylacrylamido)ethyl(2-(8-hydroxy-2-oxo-1,2-dihydroquinolin-5-yl)ethyl)carbamate (70 mg) [Step ii] and 2-(3-chlorophenyl)ethylamine (63 mg) were combined in ethanol (5.8 mL) and the resulting solution stirred at 50° C. for 18 h. The mixture was then concentrated in vacuo prior to addition of acetic acid (2 mL) followed by 33% HBr in acetic acid (3 mL). The resulting solution was stirred for 3 h then concentrated in vacuo and the residue partially purified by flash chromatography on silica (eluting with 10% methanol in dichloromethane). Product containing fractions were combined and concentrated in vacuo. The residue was further purified by reverse phase preparative HPLC (eluting with 5-40% acetonitrile in 0.1% aqueous trifluoroacetic acid). Product containing fractions were combined and concentrated in vacuo to afford the title compound as a solid (5 mg)

MS (APCI+) 539 [M+H]⁺

¹H NMR (399.825 MHz, CD₃OD) δ 8.16 (d, J=9.7 Hz, 1H), 7.41-7.35 (m, 2H), 7.30-7.22 (m, 2H), 6.98 (dd, J=22.2, 8.1 Hz, 2H), 6.66 (d, J=10.0 Hz, 1H), 3.65-3.57 (m, 3H), 3.38-3.27 (m, 4H), 3.23-3.14 (m, 8H), 2.91 (t, J=6.0 Hz, 2H), 1.88-1.82 (m, 2H), 1.79-1.74 (m, 2H), 1.70-1.63 (m, 1H), 1.56-1.26 (m, 4H), 1.22-1.09 (m, 1H).

### EXAMPLE 14

### 3-(4-Chlorophenethylamino)-N-cyclohexyl-N-(2-(2-(8-hydroxy-2-oxo-1,2-dihydroquinolin-5-yl)ethylamino)ethyl)propanamide bis(trifluoroacetate)

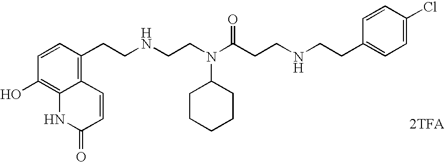

Prepared by an analogous procedure to Example 3, Step v) using benzyl 2-(N-cyclohexylacrylamido)ethyl(2-(8-hydroxy-2-oxo-1,2-dihydroquinolin-5-yl)ethyl)carbamate (100 mg) [Example 13, Step ii] in place of benzyl 2-(N-cycloheptylacrylamido)ethyl(2-(8-hydroxy-2-oxo-1,2-dihydroquinolin-5-yl)ethyl)carbamate and 2-(4-chlorophenyl)ethylamine (87 mg) in place of 2-(3-chlorophenyl)ethylamine.

MS (APCI+) 539 [M+H]⁺

¹H NMR (399.826 MHz, DMSO) δ 10.47 (s, 1H), 10.36 (s, 1H), 8.88-8.47 (m, 4H), 8.06 (d, J=9.7 Hz, 1H), 7.42-7.39 (m, 2H), 7.33-7.29 (m, 2H), 6.93-6.87 (m, 2H), 6.57 (d, J=9.7 Hz, 1H), 3.58-3.46 (m, 1H), 3.27-3.06 (m, 10H), 3.04-2.90 (m, 4H), 2.84-2.79 (m, 2H), 1.81-1.74 (m, 2H), 1.72-1.23 (m, 7H), 1.14-1.02 (m, 1H).

### EXAMPLE 15

### N-Cyclohexyl-3-(3,5-difluorophenethylamino)-N-(2-(2-(8-hydroxy-2-oxo-1,2-dihydroquinolin-5-yl)ethylamino)ethyl)propanamide bis(trifluoroacetate)

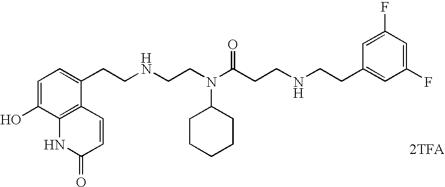

Prepared by an analogous procedure to Example 14 using 2-(3,5-difluorophenyl)ethanamine hydrochloride (91 mg) and N,N-diisopropylethylamine (0.037 mL) in place of 2-(4-chlorophenyl)ethylamine.

MS (APCI+) 541 [M+H]⁺

¹H NMR (399.825 MHz, CD₃OD) δ 8.15 (d, J=9.7 Hz, 1H), 7.01 (d, J=7.9 Hz, 1H), 6.66 (d, J=9.7 Hz, 1H), 6.97-6.90 (m, 3H), 6.87-6.79 (m, 1H), 3.64-3.58 (m, 3H), 3.3.7-3.27 (m, 4H), 3.22 (s, 4H), 3.18-3.13 (m, 2H), 3.06-3.01 (m, 2H), 2.91-2.87 (m, 2H), 1.84 (d, J=13.1 Hz, 2H), 1.74 (d, 2H), 1.70-1.63 (m, 1H), 1.56-1.33 (m, 4H), 1.26 (t, J=7.2 Hz, 1H).

### EXAMPLE 16

### N-Cyclohexyl-3-(3,4-difluorophenethylamino)-N-(2-(2-(8-hydroxy-2-oxo-1,2-dihydroquinolin-5-yl)ethylamino)ethyl)propanamide bis(trifluoroacetate)

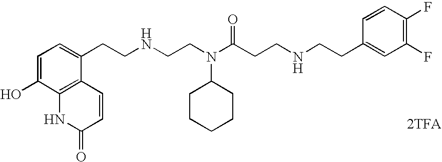

Prepared by an analogous procedure to Example 14 using 2-(3,4-difluorophenyl)ethanamine (108 mg) in place of 2-(4-chlorophenyl)ethylamine.

MS (APCI+) 541 [M+H]⁺

¹H NMR (399.825 MHz, CD₃OD) δ 8.16 (d, J=9.7 Hz, 1H), 7.26-7.16 (m, 2H), 7.10-7.05 (m, 1H), 6.98 (dd, J=21.4, 8.1 Hz, 2H), 6.66 (d, J=9.7 Hz, 1H), 3.64-3.58 (m, 3H), 3.35-3.26 (m, 4H), 3.22 (s, 4H), 3.15 (t, J=5.8 Hz, 2H), 3.01 (t, J=7.9 Hz, 2H), 2.89 (t, J=6.2 Hz, 2H), 1.88-1.80 (m, 2H), 1.79-1.73 (m, 2H), 1.69-1.63 (m, 1H), 1.56-1.32 (m, 4H), 1.27-1.09 (m, 1H).

### EXAMPLE 17

### N-Cyclohexyl-3-(2,5-difluorophenethylamino)-N-(2-(2-(8-hydroxy-2-oxo-1,2-dihydroquinolin-5-yl)ethylamino)ethyl)propanamide bis(trifluoroacetate)

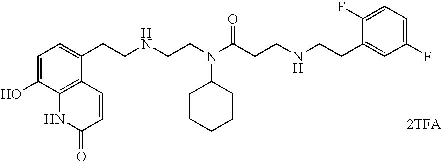

Prepared by an analogous procedure to Example 14 using 2-(2,5-difluorophenyl)ethanamine hydrochloride (91 mg) and N,N-diisopropylethylamine (0.08 mL) in place of 2-(4-chlorophenyl)ethylamine.

MS (APCI+) 541 [M+H]⁺

¹H NMR (399.825 MHz, CD₃OD) δ 8.16 (d, J=9.7 Hz, 1H), 7.15-7.08 (m, 2H), 7.05-6.99 (m, 3H), 6.97 (s, 1H), 3.64-3.56 (m, 3H), 3.37-3.29 (m, 4H), 3.22 (s, 4H), 3.18-3.13 (m, 2H), 3.09-3.04 (m, 2H), 2.92-2.87 (m, 2H), 1.88-1.82 (m, 2H), 1.79-1.73 (m, 2H), 1.70-1.64 (m, 2H), 1.55-1.11 (m, 4H).

### EXAMPLE 18

### N-Cyclohexyl-3-(3-difluorophenethylamino)-N-(2-(2-(8-hydroxy-2-oxo-1,2-dihydroquinolin-5-yl)ethylamino)ethyl)propanamide bis(trifluoroacetate)

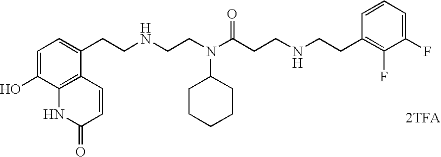

Prepared by an analogous procedure to Example 14 using 2-(2,3-difluorophenyl)ethanamine hydrochloride (97 mg) and N,N-diisopropylethylamine (0.07 mL) in place of 2-(4-chlorophenyl)ethylamine.

MS (APCI+) 541 [M+H]⁺

¹H NMR (399.825 MHz, CD₃OD) δ 8.16 (d, J=9.7 Hz, 1H), 7.22-7.09 (m, 3H), 6.98 (d, J=14.4 Hz, 2H), 6.66 (d, J=9.7 Hz, 1H), 3.65-3.57 (m, 3H), 3.38-3.26 (m, 4H), 3.22 (s, 4H), 3.18-3.09 (m, 4H), 2.90 (t, 2H), 1.88-1.80 (m, 2H), 1.79-1.73 (m, 2H), 1.69-1.63 (m, 1H), 1.56-1.32 (m, 4H), 1.22-1.09 (m, 1H).

### EXAMPLE 19

### N-Cyclohexyl-3-(3-fluorophenethylamino)-N-(2-(2-(8-hydroxy-2-oxo-1,2-dihydroquinolin-5-yl)ethylamino)ethyl)propanamide

### Step i) tert-Butyl 3-(3-fluorophenethylamino)propanoate

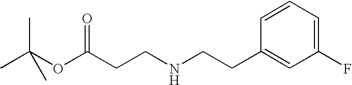

tert-Butyl acrylate (4.91 g, 5.61 mL) was added to a solution of 3-fluorophenethylamine (5.33 g, 5 mL) in ethanol (200 mL) and the reaction was stirred at ambient temperature for 2 days. The mixture was concentrated in vacuo to give the subtitled compound as an oil (9.6 g)

¹H NMR (299.946 MHz, CDCl₃) δ 7.28-7.20 (m, 1H), 7.01-6.85 (m, 3H), 2.88-2.74 (m, 6H), 2.41 (t, J=6.5 Hz, 2H), 1.42 (s, 9H)

### Step ii) tert-Butyl 3-((benzyloxycarbonyl)(3-fluorophenethyl)amino)propanoate

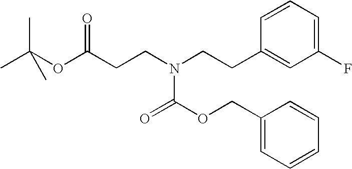

Benzyl chloroformate (6.66 g) was added to a cooled (˜5° C.) solution of tert-butyl 3-(3-fluorophenethylamino)propanoate (9.5 g) [Step i] and triethylamine (4.31 g) in dichloromethane (100 mL) over a period of 5 minutes. The mixture was allowed to warm to ambient temperature and stirred for 18 h, then concentrated in vacuo. The residue was purified by flash chromatography on silica (eluting with 10% ethyl acetate in iso-hexane) to give the subtitled product as an oil (11.5 g).

¹H NMR (399.826 MHz, DMSO) δ 7.37-7.22 (m, 6H), 7.06-6.91 (m, 3H), 5.05 (s, 2H), 3.46 (t, J=7.3 Hz, 2H), 3.39 (t, J=7.0 Hz, 2H), 2.81 (t, J=7.4 Hz, 2H), 2.41 (t, J=7.2 Hz, 2H), 1.38 (s, 9H)

### Step iii) 3-((Benzyloxycarbonyl)(3-fluorophenethyl)amino)propanoic acid

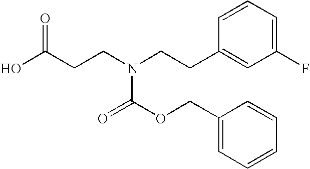

Trifluoroacetic acid (50 mL) was added to a stirred solution of tert-butyl 3-((benzyloxycarbonyl)(3-fluorophenethyl)amino)propanoate (11.5 g) [Step ii] in dichloromethane. After 2 h, the solution was concentrated in vacuo and the oily residue azeotroped twice with toluene to afford the subtitled compound as a viscous oil which solidified on standing (10.5 g).

¹H NMR (399.826 MHz, DMSO) δ 7.41-7.27 (m, 6H), 7.08-6.94 (m, 3H), 5.04 (d, J=25.9 Hz, 2H), 3.45 (t, J=7.4 Hz, 2H), 3.38 (s, 2H), 2.84-2.76 (m, 2H), 2.45 (t, J=7.0 Hz, 2H)

### Step iv) Benzyl 3-(cyclohexyl(2,2-dimethoxyethyl)amino)-3-oxopropyl(3-fluorophenethyl)carbamate

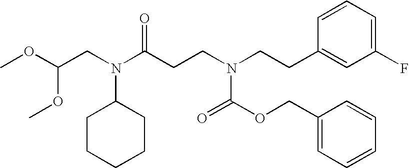

Oxalyl chloride (1.64 mL) was added dropwise over 10 min to a solution of 3-((benzyloxycarbonyl)(3-fluorophenethyl)amino)propanoic acid (5 g) [Step iii] in dichloromethane (50 mL) containing dimethylformamide (2 drops). The mixture was stirred at ambient temperature for 1 h, concentrated in vacuo and redissolved in dichloromethane (25 mL). The solution was added dropwise to a preformed mixture of N-(2,2-dimethoxyethyl)cyclohexanamine (2.71 g) [Example 2; Step i] and triethylamine (3.0 mL) in dichloromethane (25 mL) at 0° C. under nitrogen. The mixture was stirred at 0° C. for 1 h, then water (25 mL) was added and the layers were separated. The organic layer was washed with 2 M hydrochloric acid, saturated aqueous sodium hydrogen carbonate and brine before being dried over anhydrous magnesium sulfate, filtered and concentrated in vacuo to give the subtitled compound as an oil (7.45 g).

¹H NMR (399.826 MHz, DMSO) δ 7.39-7.24 (m, 6H), 7.01-6.93 (m, 3H), 5.05 (s, 2H), 4.43-4.35 (m, 1H), 3.50-3.38 (m, 3H), 3.31-3.21 (m, 8H), 3.03-2.96 (m, 2H), 2.85-2.78 (m, 2H), 2.59-2.53 (m, 2H), 1.78-1.68 (m, 2H), 1.62-1.39 (m, 5H), 1.32-1.19 (m, 2H), 1.13-1.00 (m, 1H).

MS: APCI (+ve) 515 [M+H]⁺

### Step v) Benzyl 3-(cyclohexyl(2-oxoethyl)amino)-3-oxopropyl(3-fluorophenethyl)carbamate

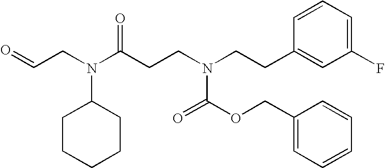

p-Toluenesulfonic acid monhydrate (0.492 g) was added to a solution of benzyl 3-(cyclohexyl(2,2-dimethoxyethyl)amino)-3-oxopropyl(3-fluorophenethyl)carbamate (0.444 g) [Step iv] in dichloromethane (10 mL). The resulting solution was stirred at ambient temperature for 4.5 h, then diluted with dichloromethane and washed with saturated sodium hydrogen carbonate, water and saturated brine. The organic phase was dried over is anhydrous sodium sulfate, filtered and concentrated in vacuo to afford the subtitled compound (0.371 g).

MS (APCI+) 469 [M+H]⁺

### Step yl) 5-(2-Aminoethyl)-8-(benzyloxy)quinolin-2(1H)-one

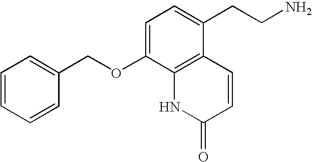

### 5-(2-Amino-1-(tert-butyldimethylsilyloxy)ethyl)-8-(benzyloxy)quinolin-2(1H)-one (3 g) was dissolved in dichloromethane (30 mL) and cooled in ice. Trifluoroacetic anhydride (2.22 mL) was added dropwise and the reaction was stirred at ambient temperature for 2 hr. Trifluoroacetic acid (30 mL), followed by triethylsilane (4.79 mL, 30 mmol) were added and the reaction was stirred at ambient temperature overnight. Further triethylsilane (4.79 mL) was added and the reaction was left at ambient temperature for a further 4 days, prior to concentration in vacuo. The residue was purified by flash chromatography on silica (eluting with 3% methanol in dichloromethane). Product containing fractions were combined and evaporated and the residue was crystallised from ethyl acetate to afford a white solid (1.75 g). This material was combined with potassium carbonate (5 g) in a mixture of methanol (15 mL), water (15.00 mL) and tetrahydrofuran (15.00 mL) and the resulting solution heated at 50° C. for 18 h. The reaction was concentrated to dryness in vacuo and the residue was extracted with hot methanol. The extracts were cooled, filtered and evaporated to give a white sticky solid which was dissolved in water and loaded onto an SCX cartridge. The cartridge was washed with water and methanol-water and the product was eluted with 10% concentrated aqueous ammonia in methanol. The resulting solution was concentrated and dried in vacuo to afford the subtitled compound as an oil, which solidified on standing to a cream solid (1.3 g).

¹H NMR (399.826 MHz, DMSO) δ 8.08 (d, J=10.0 Hz, 1H), 7.57 (d, J=7.2 Hz, 2H), 7.38 (t, J=7.3 Hz, 2H), 7.31 (t, J=7.3 Hz, 1H), 7.13 (d, J=8.2 Hz, 1H), 6.92 (d, J=8.2 Hz, 1H), 6.54 (d, J=9.7 Hz, 1H), 5.27 (s, 2H), 2.87 (t, J=7.2 Hz, 2H), 2.72 (t, J=7.4 Hz, 2H)

### Step vii) Benzyl 3-(cyclohexyl(2-(2-(8-hydroxy-2-oxo-1,2-dihydroquinolin-5-yl)ethylamino)ethyl)amino)-3-oxopropyl(3-fluorophenethyl)carbamate

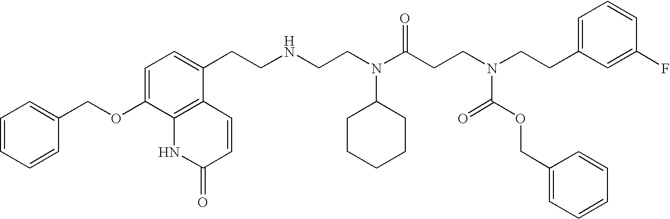

Benzyl 3-(cyclohexyl(2-oxoethyl)amino)-3-oxopropyl(3-fluorophenethyl)carbamate (0.24 g) [Step v], 5-(2-aminoethyl)-8-(benzyloxy)quinolin-2(1H)-one (0.27 g) [Step vi] and anhydrous magnesium sulfate (0.34 g) were combined in dichloromethane (2.6 mL). After stirring for 1 h, sodium triacetoxyborohydride (0.197 g) was added and the mixture was stirred at ambient temperature for 18 h before being partitioned between dichloromethane and saturated aqueous sodium hydrogen carbonate solution. The phases were separated and the organic phase washed with water, and brine, then dried over anhydrous magnesium sulfate, filtered and concentrated in vacuo. The residue was purified by flash chromatography on silica (eluting with 20% methanol in ethyl acetate, containing 1% 7 N ammonia in methanol). Product containing fractions were concentrated in vacuo and further purified by reverse phase preparative HPLC (eluting with 60-70% acetonitrile in aqueous ammonium actetate) to afford the subtitled compound as a gum (46 mg).

MS 747 [M+H]+

### Step viii) N-Cyclohexyl-3-(3-fluorophenethylamino)-N-(2-(2-(8-hydroxy-2-oxo-1,2-dihydroquinolin-5-yl)ethylamino)ethyl)propanamide

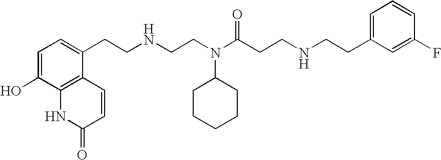

A pre-moistened sample of 110% palladium on carbon (18 mg) was added to a solution of benzyl 3-(cyclohexyl(2-(2-(8-hydroxy-2-oxo-1,2-dihydroquinolin-5-yl)ethylamino)ethyl)amino)-3-oxopropyl(3-fluorophenethyl)carbamate (30 mg) [Step vii] in ethanol (3 mL) and the mixture was hydrogenated with 3 atm of hydrogen gas for 18 h. The mixture was filtered through Hyflo and the filtrate concentrated in vacuo to afford the title compound as an pale yellow solid (25 mg).

MS (APCI+) 523 [M+H]⁺

¹H NMR (399.826 MHz, DMSO) δ 8.03 (dd, J=9.9, 6.5 Hz, 1H), 7.32-7.25 (m, 1H), 7.06-6.94 (m, 3H), 6.90-6.80 (m, 2H), 6.48 (dd, J=9.7, 1.0 Hz, 1H), 4.07-3.98 (m, 0.5H), 3.57-3.48 (m, 0.5H), 3.21-3.11 (m, 2H), 2.90-2.82 (m, 2H), 2.75-2.38 (m, 12H), 1.77-1.67 (m, 2H), 1.61-1.53 (m, 2H), 1.49-1.16 (m, 5H), 1.12-0.99 (m, 1H).

### EXAMPLE 20

### N-Cyclohexyl-3-(3-fluorophenethylamino)-N-(2-(2-(7-hydroxy-2-oxoindolin-4-yl)ethylamino)ethyl)propanamide dihydrobromide

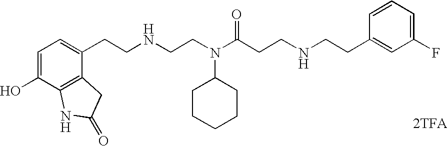

4-(2-Aminoethyl)-7-hydroxyindolin-2-one hydrobromide (0.374 g) [J. Med. Chem., 1986, 29, 939-47], tetrahydrofuran (13 mL), triethylamine (0.4 mL), water (4 mL), acetic acid (0.2 mL) and NMP (4 mL) were combined. Benzyl 3-(cyclohexyl(2-oxoethyl)amino)-3-oxopropyl(3-fluorophenethyl)carbamate (0.642 g) [Example 19, Step v] was added as a solution in tetrahydrofuran (4 mL) and the mixture was stirred for 1 h, prior to addition of sodium cyanoborohydride (0.258 g). Stirring was continued for 18 h and the reaction mixture was then loaded onto an SCX cartridge. The solid phase was washed with a methanol/dichloromethane mixture and the product was eluted with 10% ammonia in methanol and the eluent was concentrated in vacuo. The residue was suspended in acetic acid (5 mL) and 33% hydrogen bromide in acetic acid (5 mL) was added. The mixture was stirred at ambient temperature for 1 h and then diluted with toluene and concentrated in vacuo to give an oily residue. This was purified by reverse phase preparative HPLC (eluting with 5-95% acetonitrile in 0.2% aqueous trifluoroacetic acid. The fractions containing the desired compound were evaporated to dryness to give a solid, which was dried at 45° C. under vacuum for 18 h to give the title compound (60 mg)

MS (APCI+) 511 [M+H]⁺

¹H NMR (399.826 MHz, DMSO) δ 10.15 (s, 1H), 9.46-9.42 (m, 1H), 8.82-8.43 (m, 4H), 7.42-7.36 (m, 1H), 7.18-7.07 (m, 3H), 6.70-6.62 (m, 2H), 3.57-3.45 (m, 5H), 3.29-3.05 (m, 6H), 3.01-2.94 (m, 4H), 2.85-2.69 (m, 4H), 1.80-1.04 (m, 10H).

### EXAMPLE 21

### N-Cyclohexyl-N-(2-(3-formamido-4-hydroxyphenethylamino)ethyl)-3-(phenethylamino)propanamide

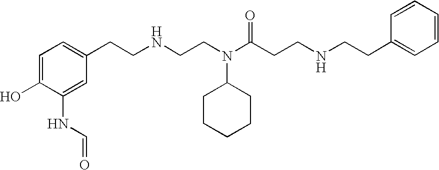

### Step i) Benzyl 3-(cyclohexyl(2-oxoethyl)amino)-3-oxopropyl(phenethyl)carbamate

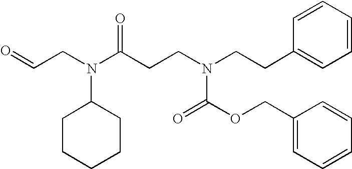

Prepared by an analogous procedure to Example 19, Steps i-v, except using 2-phenylethylamine in place of 3-fluorophenethylamine.

MS (APCI+) 541 [M+H]⁺

### Step ii) Benzyl 3-((2-(4-(benzyloxy)-3-nitrophenethylamino)ethyl)(cyclohexyl)amino)-3-oxopropyl(phenethyl)carbamate

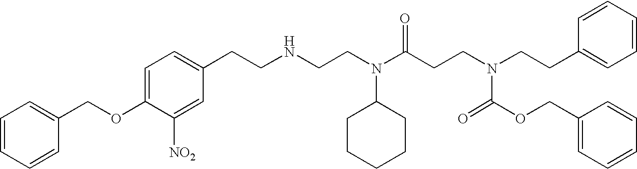

A solution of 2-(4-(benzyloxy)-3-nitrophenyl)ethanamine (0.564 g) [DE2227022] dissolved in dichloromethane (10 mL) was added to a solution of benzyl 3-(cyclohexyl(2-oxoethyl)amino)-3-oxopropyl(phenethyl)carbamate (0.683 g) [Step i] in dichloromethane (5 mL). Anhydrous sodium sulfate (˜200 mg) was then added. The resulting suspension was stirred at ambient temperature for 2 h then sodium triacetoxyborohydride (0.643 g, 3.03 mmol) was added and the reaction mixture stirred overnight. It was then diluted with dichloromethane and washed with saturated sodium hydrogen carbonate, water and saturated brine. The organic phase was dried over anhydrous sodium sulfate, filtered and concentrated in vacuo to afford the subtitled product as a yellow gum (1.032 g), which was used without further purification.

MS (APCI+) 707 [M+H]⁺

### Step iii) Benzyl 4-(benzyloxy)-3-nitrophenethyl(2-(3-((benzyloxycarbonyl)(phenethyl)amino)-N-cyclohexylpropanamido)ethyl)carbamate

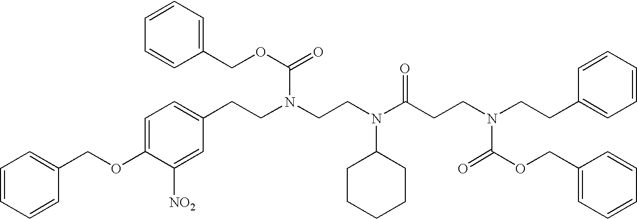

Triethylamine (0.244 mL) and benzyl chloroformate (0.250 mL, 1.75 mmol) were added to a cooled (0° C.) solution of benzyl 3-((2-(4-(benzyloxy)-3-nitrophenethylamino)ethyl)(cyclohexyl)amino)-3-oxopropyl(phenethyl)carbamate (1.032 g) [Step ii] in dichloromethane (20 mL). The reaction mixture was allowed to warm to ambient temperature and stirred for 18 h, then diluted with dichloromethane and washed with 2M hydrochloric acid (×2), water and saturated brine. The organic phase was dried over anhydrous sodium sulfate, filtered and concentrated in vacuo. The crude product was purified by flash chromatography on silica (eluting with 30-60% ethyl acetate in isohexane). Pure fractions were concentrated in vacuo to dryness to afford the subtitled compound as a yellow gum (0.627 g, 51.1%).

MS (APCI+) 841 [M+H]⁺

¹H NMR (399.826 MHz, 90° C., DMSO) δ 7.64-7.60 (m, 1H), 7.42-7.13 (m, 22H), 5.24-5.20 (m, 2H), 5.05-5.00 (m, 4H), 3.47-3.37 (m, 5H), 3.24-3.19 (m, 4H), 3.20-3.15 (m, 4H), 2.83-2.74 (m, 4H), 1.71-1.16 (m, 10H).

### Step iv) Benzyl 3-amino-4-(benzyloxy)phenethyl(2-(3-((benzyloxycarbonyl)(phenethyl)amino)-N-cyclohexylpropanamido)ethyl)carbamate

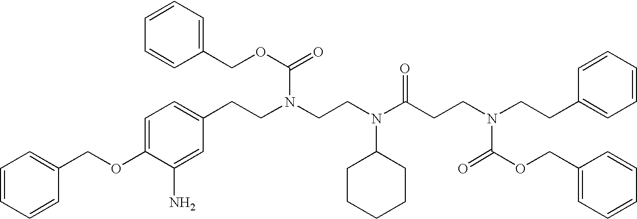

Benzyl 4-(benzyloxy)-3-nitrophenethyl(2-(3-((benzyloxycarbonyl)(phenethyl)amino)-N-cyclohexylpropanamido)ethyl)carbamate (0.470 g) [Step iii] was dissolved in methanol (20 mL) and ammonium chloride (0.299 g) added, followed by zinc dust (0.365 g). The reaction mixture was heated at 65° C. for 2 h then allowed to cool, filtered through Celite and the volatiles evaporated. The residue was diluted with ethyl acetate and washed with water and saturated brine. The organic portion was dried over anhydrous sodium sulfate, filtered and concentrated in vacuo to afford the crude subtitled product as a colourless oil (0.437 g) which was used without further purification.

MS (APCI+) 811 [M+H]⁺

### Step v) Benzyl 4-(benzyloxy)-3-formamidophenethyl(2-(3-((benzyloxycarbonyl)(phenethyl)amino)-N-cyclohexylpropanamido)ethyl)carbamate

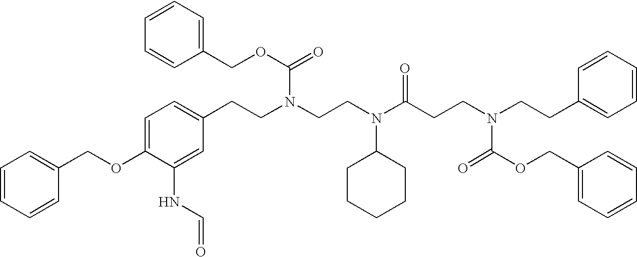

Formic acid (0.34 mL) and acetic anhydride (0.56 mL) were mixed together and stirred for 20 minutes, before 10 μL was added dropwise to a cooled (0° C.) solution of benzyl 3-amino-4-(benzyloxy)phenethyl(2-(3-((benzyloxycarbonyl)(phenethyl)amino)-N-cyclohexylpropanamido)ethyl)carbamate (42.0 mg) [Step iv] in tetrahydrofuran (1 mL). After 5 h, the reaction mixture was diluted with ethyl acetate and washed with saturated sodium hydrogen carbonate (×2), water and saturated brine. The organic phase was dried over anhydrous sodium sulfate, filtered and evaporated to afford crude product as a colourless oil (27 mg), which was used without further purification.

MS (APCI+) 839 [M+H]⁺

### Step vi) N-Cyclohexyl-N-(2-(3-formamido-4-hydroxyphenethylamino)ethyl)-3-(phenethylamino)propanamideN-cyclohexyl-N-(2-(3-formamido-4-hydroxyphenethylamino)ethyl)-3-(phenethylamino)propanamide

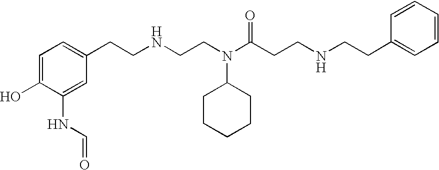

10% Palladium on carbon (10.00 mg) was added to a solution of benzyl 4-(benzyloxy)-3-formamidophenethyl(2-(3-((benzyloxycarbonyl)(phenethyl)amino)-N-cyclohexylpropanamido)ethyl)carbamate (27.0 mg) [Step v] in ethanol (5 mL). The reaction mixture was hydrogenated at 2 bar for 5 h then filtered and the solvent evaporated. The crude product was purified by reverse phase preparative HPLC (eluting with 25-95% acetonitrile in aqueous 0.2% trifluoroacetic acid). The fractions containing the desired compound were evaporated to dryness to afford the title compound as a white foam (3 mg).

MS (APCI+) [M+H]⁺ 481.3

¹H NMR (399.826 MHz, 90° C., DMSO) δ 7.35-7.23 (m, 6H), 6.85-6.80 (m, 2H), 3.57-3.49 (m, 3H), 3.26-2.71 (m, 14H), 1.81-1.06 (m, 10H).

### EXAMPLE 22

### N-Cyclohexyl-N-(2-(3,4-dihydroxyphenethylamino)ethyl)-3-(phenethylamino)propanamide bis(trifluoroacetate)

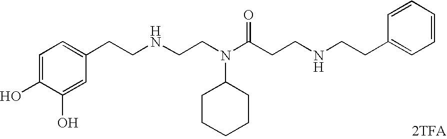

Benzyl 3-(cyclohexyl(2-oxoethyl)amino)-3-oxopropyl(phenethyl)carbamate (0.5 M solution in NMP) (1.0 mL) [Example 21, Step 1], followed by sodium triacetoxyborohydride (0.159 g) was added to a solution of 3-hydroxytyramine hydrochloride (0.095 g) and triethylamine (0.070 mL) in NMP (1 mL) and water (0.1 mL) and the mixture stirred at 22° C. for 30 minutes, then stored in the freezer for 18 h. On rewarming to 22° C., the mixture was diluted with water (10 mL) and ethyl acetate (10 mL). Brine (2 mL) was added and the phases were then separated. The aqueous phase was extracted with further ethyl acetate (20 mL) and the combined organic extracts were washed with brine (20 mL), dried over anhydrous sodium sulfate, filtered and concentrated in vacuo. 33% Hydrogen bromide n acetic acid (2 mL) was added to the crude material and the mixture stood for 2 h. The solution was then concentrated in vacuo and the residue dissolved in 2 mL of 1:1 acetonitrile/water. The solution was filtered and the plug washed with methanol (1 mL). The combined filtrate was purified by reverse phase preparative HPLC (eluting with 10-40% acetonitrile in aqueous 0.2% trifluoroacetic acid). The fractions containing the desired compound were evaporated to dryness to afford a colorless gum, which was dried in vacuo for 2 days to afford the title compound as a white solid (102 mg).

MS (APCI+) 454 [M+H]⁺

¹H NMR (399.826 MHz, DMSO) d 9.01-8.52 (m, 6H), 7.39-7.18 (m, 5H), 6.73-6.56 (m, 2H), 6.53-6.42 (m, 1H), 3.59-3.32 (m, 3H), 3.27-2.65 (m, 14H), 1.83-1.21 (m, 9H), 1.15-1.01 (m, 1H).

### EXAMPLE 23

### N-Cyclohexyl-N-(2-(2-(5-hydroxy-3-oxo-3,4-dihydro-2H-benzo[b][1,4]oxazin-8-yl)ethylamino)ethyl)-3-(phenethylamino)propanamide bis(trifluoroacetate)

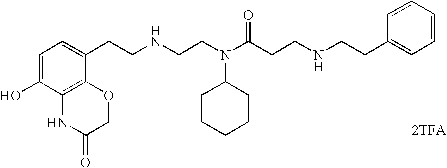

### Step i) 1-(2,4-Dihydroxy-3-nitrophenyl)ethanone

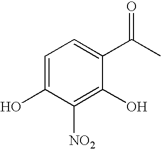

### 2-Nitrobenzene-1,3-diol (24.5 g) was added portionwise over 15 minutes to a vigorously stirred solution of aluminum chloride (46.3 g) in nitrobenzene (325 mL). Acetic anhydride (15.65 mL) was then added dropwise to the mixture over a further 15 minutes and the mixture then heated at 100° C. for 5 h. The reaction was cooled to ambient temperature and carefully quenched with ice cold 2M hydrochloric acid (300 mL). The mixture was extracted with ether (2×500 mL) and the combined ether extracts then extracted with 2 M aqueous sodium hydroxide (2×400 mL). The combined basic extracts were washed with ether (4×500 mL) and then acidified to pH 1 with 2M hydrochloric acid (700 mL). The resulting precipitate was filtered off, washed with water, and dried under vacuum at 40° C. to afford the subtitled compound as a yellow/brown solid (29.5 g).

¹H NMR (399.826 MHz, DMSO) δ 13.32 (s, 1H), 12.31 (s, 1H), 7.98 (d, J=9.2 Hz, 1H), 6.63 (d, J=28.2 Hz, 1H), 2.59 (s, 3H).

### Step ii) 1-(4-(Benzyloxy)-2-hydroxy-3-nitrophenyl)ethanone

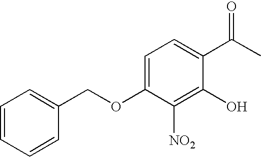

Lithium tert-butoxide (4.06 g) was added to a stirred solution of 1-(2,4-dihydroxy-3-nitrophenyl)ethanone (10 g) [Step i] in DMF (100 mL), under nitrogen, whilst maintaining the internal temperature below 30° C. After stirring for a further 10 minutes at ambient temperature, benzyl bromide (6.03 mL) was added and the mixture stirred for a further 20 h. Further benzyl bromide (3 mL) was added and the mixture stirred for 24 h. The reaction was quenched with water (300 mL), 1 M aqueous sodium hydroxide (50 mL) was added and the mixture was washed with ether (2×300 mL), (filtered through celite to aid separation). The basic solution was cooled in ice/water, acidified with ice cold 2 M hydrochloric acid (200 mL) and the resulting precipitate filtered off, washed with water and dried to afford a light brown solid. The solid was slurried with ethanol (100 mL) for 1 h and the solid filtered off, washed with cold ethanol (20 mL), and dried under vacuum at 40° C. to afford the subtitled compound as a light brown solid (6.8 g).

¹H NMR (399.826 MHz, DMSO) δ 13.04 (s, 1H), 8.14 (d, J=9.2 Hz, 1H), 7.45-7.32 (m, 5H), 7.01 (d, J=9.2 Hz, 1H), 5.42 (s, 2H), 2.64 (s, 3H).

### Step iii) 1-(3-Amino-4-(benzyloxy)-2-hydroxyphenyl)ethanone

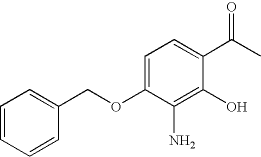

Zinc dust (5.5 g) was added portionwise to a suspension of 1-(4-(benzyloxy)-2-hydroxy-3-nitrophenyl)ethanone (5.5 g) [Step ii] in AcOH (55 mL) over 15 minutes, whilst maintaining the internal temperature below 40° C. with an ice bath. The mixture was allowed to attain ambient temperature and stirred for a further 2 h. The mixture was filtered through celite (caution gets hot, do not allow to dry), washed with acetic acid, and the filtrate poured onto ice/water (500 mL). The resulting precipitate was filtered off, washed with water, and dried under vacuum at 40° C. to afford the subtitled compound as a light brown solid (4.8 g).

¹H NMR (299.947 MHz, DMSO) δ 7.53 (m, 2H), 7.48-7.33 (m, 3H), 7.28 (d, J=9.0 Hz, 1H), 6.72 (d, J=9.0 Hz, 1H), 5.29 (s, 2H), 2.59 (s, 3H).

### Step iv) 8-Acetyl-5-(benzyloxy)-2H-benzo[b][1,4]oxazin-3(4H)-one

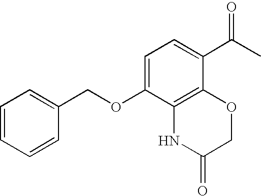

2-Chloroacetyl chloride (1.771 mL) was added dropwise to a stirred mixture of 1-(3-amino-4-(benzyloxy)-2-hydroxyphenyl)ethanone (5.2 g) [Step iii] and sodium hydrogen carbonate (3.74 g) in DMF (30 mL) and then stirred for a further 2 h. Cesium carbonate (7.90 g) was added and heated at 100° C. for 20 h. The mixture was cooled to ambient temperature, quenched with water (500 mL), extracted with ethyl acetate (2×200 mL), washed with water (3×300 mL) and brine, dried over anhydrous sodium sulfate, filtered and evaporated in vacuo. The solid residue was treated with ether, filtered and dried to afford the subtitled compound as a beige solid (5.7 g).

¹H NMR (399.826 MHz, DMSO) δ 10.33 (s, 1H), 7.55 (m, 2H), 7.39 (m, 2H), 7.34 (d, J=8.8 Hz, 1H), 7.33 (m, 1H), 6.89 (d, J=9.2 Hz, 1H), 5.27 (s, 2H), 4.67 (s, 2H), 3.32 (s 3H).

### Step v) 5-(Benzyloxy)-8-(2-chloroacetyl)-2H-benzo[b][1,4]oxazin-3(4H)-one

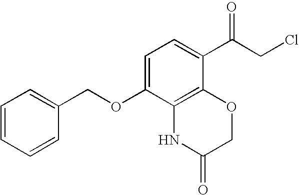

Benzyltrimethylammonium dichloroiodate (14.17 g) was added to a stirred solution of 8-acetyl-5-(benzyloxy)-2H-benzo[b][1,4]oxazin-3(4H)-one (5.5 g) [Step iv] in a mixture of dichloromethane (100 mL), AcOH (33 mL) and water (5.5 mL) and the reaction mixture stirred at 65° C. for 20 h. The reaction was cooled to ambient temperature, treated with aqueous sodium bisulphite (5.78 g in 100 mL) and stirred for a further 30 minutes. The mixture was diluted with diethylether (200 mL) and the resulting solid filtered off, washed with water and further diethylether, and dried under vacuum at 40° C. to afford the subtitled to compound as a light brown solid (5.6 g).

¹H NMR (299.947 MHz, DMSO) δ 10.41 (s, 1H), 7.55 (m, 2H), 7.44 (d, J=9.4 Hz, 1H), 7.39 (m, 2H), 7.32 (m, 1H), 6.95 (d, J=9.4 Hz, 1H), 5.30 (s, 2H), 4.96 (s, 2H), 4.69 (s, 2H).

### Step vi) 8-(2-Azidoacetyl)-5-(benzyloxy)-2H-benzo[b][1,4]oxazin-3(4H)-one

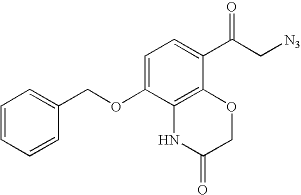

Sodium azide (1.176 g) was added to a suspension of 5-(benzyloxy)-8-(2-chloroacetyl)-2H-benzo[b][1,4]oxazin-3(4H)-one (4.8 g) [Step v] in DMF (50 mL) and stirred for 2 h. The mixture was poured onto ice/water and the resulting solid filtered off, washed with water and dried under vacuum at 40° C. to afford the subtitled compound as a light brown solid (4.6 g).

¹H NMR (299.947 MHz, DMSO) δ 10.42 (s, 1H), 7.55 (m, 2H), 7.48 (m, 1H), 7.43-7.29 (m, 3H), 6.97 (m, 1H), 5.31 (s, 2H), 4.69 (s, 2H), 4.63 (s, 2H).

### Step vii) 8-(2-Aminoethyl)-5-hydroxy-2H-benzo[b][1,4]oxazin-3(4H)-one hydrochloride

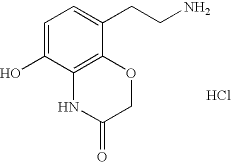

A slurry of 10% palladium on carbon (1 g) in acetic acid (20 mL) was added to a partial solution of 8-(2-azidoacetyl)-5-(benzyloxy)-2H-benzo[b][1,4]oxazin-3(4H)-one (5.65 g) [Step vi] in acetic acid (280 mL). Concentrated hydrochloric acid (14.34 mL) was then added, and the mixture hydrogenated at 5 bar for 6 h. Water (50 mL) was added to dissolve any solid, followed by further 10% palladium on carbon (1 g) and the mixture hydrogenated at 5 bar for a further 20 h. Further 10% palladium on carbon (1 g) was added and the mixture hydrogenated for a further 20 h. The mixture was filtered through celite and the filtrate evaporated in vacuo, and azeotroped with acetonitrile. The solid residue was treated with ether, isolated by filtration and dried to afford the subtitled compound as a white solid (2.2 g).

¹H NMR (299.947 MHz, DMSO) δ 9.94 (s, 1H), 9.87 (s, 1H), 7.99-7.82 (m, 3H), 6.66 (d, J=8.0 Hz, 1H), 6.49 (d, J=8.0 Hz, 1H), 4.54 (s, 2H), 2.91 (m, 2H), 2.76 (m, 2H).

### Step viii) N-Cyclohexyl-N-(2-(2-(5-hydroxy-3-oxo-3,4-dihydro-2H-benzo[b][1,4]oxazin-8-yl)ethylamino)ethyl)-3-(phenethylamino)propanamide bis(trifluoroacetate)

Prepared by an analogous procedure to Example 22, using 8-(2-Aminoethyl)-5-hydroxy-2H-benzo[b][1,4]oxazin-3(4H)-one hydrochloride [Step vi] in place of 3-hydroxytyramine hydrochloride.

MS (APCI+) 509 (M+H)⁺

¹H NMR (299.947 MHz, DMSO) δ 9.94 (s, 1H), 9.89 (s, 1H), 8.63-8.46 (m, 4H), 7.35 (m, 2H), 7.27 (m, 3H), 6.66 (m, 1H), 6.48 (m, 1H), 4.54 (s, 2H), 3.66-3.36 (m, 3H), 3.21 (m, 4H), 3.07 (m, 2H), 2.94 (m, 4H), 2.79 (m, 4H), 1.83-1.02 (m, 10H).

### EXAMPLE 24

### N-Cycloheptyl-N-(2-(2-(5-hydroxy-3-oxo-3,4-dihydro-2H-benzo[b][1,4]oxazin-8-yl)ethylamino)ethyl)-3-(phenethylamino)propanamide bis(trifluoroacetate)

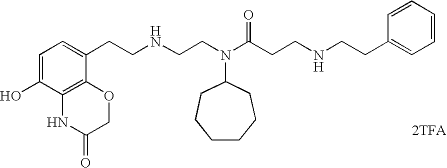

### Step i) N-(2,2-Dimethoxyethyl)cycloheptylamine

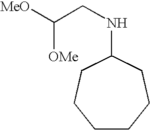

Prepared by an analogous procedure to Example 2, Step ii, using cycloheptylamine in place of cyclohexylamine.

¹H NMR (299.946 MHz, CDCl₃) δ 4.47 (t, J=5.6 Hz, 1H), 3.39 (s, 6H), 2.73 (d, J=5.6 Hz, 2H), 2.67-2.55 (m, 1H), 1.90-1.25 (m, 12H).

### Step ii) Benzyl 3-(cycloheptyl(2-oxoethyl)amino)-3-oxopropyl(phenethyl)carbamate

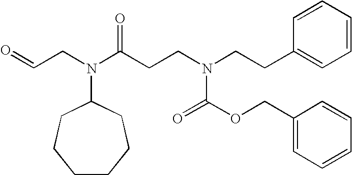

Prepared by an analogous procedure to Example 19, Steps i-v, except using 2-phenylethylamine in place of 3-fluorophenethylamine (in Step i) and N-(2,2-dimethoxyethyl)cycloheptylamine in place of N-(2,2-dimethoxyethyl)cyclohexylamine (in Step iv).

¹H NMR (299.946 MHz, CDCl₃) δ 9.43 (s, 1H), 7.42-7.05 (m, 10H), 5.20-5.03 (m, 2H), 3.88-3.77 (m, 2H), 3.57-3.45 (m, 4H), 3.40-3.36 (m, 1H), 2.92-2.75 (m, 2H), 2.72-2.47 (m, 2H), 1.85-1.30 (m, 12H).

### Step iii) Benzyl 3-(cycloheptyl(2-(2-(5-hydroxy-3-oxo-3,4-dihydro-2H-benzo[b][1,4]oxazin-8-yl)ethylamino)ethyl)amino)-3-oxopropyl(phenethyl)carbamate

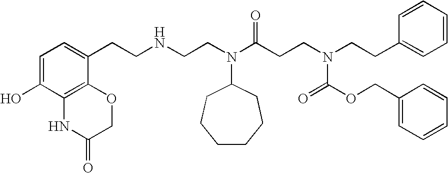

Prepared by an analogous procedure to Example 23, using 8-(2-Aminoethyl)-5-hydroxy-2H-benzo[b][1,4]oxazin-3(4H)-one hydrochloride [Example 23, Step vii] in place of 3-hydroxytyramine hydrochloride and benzyl 3-(cycloheptyl(2-oxoethyl)amino)-3-oxopropyl(phenethyl)carbamate [Step ii] in place of benzyl 3-(cyclohexyl(2-oxoethyl)amino)-3-oxopropyl(phenethyl)carbamate, but without treatment with 33% HBr in acetic acid, or purification with reverse phase HPLC.

MS: (ES+): 657 (M+H)+

### Step iv) N-Cycloheptyl-N-(2-(2-(5-hydroxy-3-oxo-3,4-dihydro-2H-benzo[b][1,4]oxazin-8-yl)ethylamino)ethyl)-3-(phenethylamino)propanamide bis(trifluoroacetate)

A solution of benzyl 3-(cycloheptyl(2-(2-(5-hydroxy-3-oxo-3,4-dihydro-2H-benzo[b][1,4]oxazin-8-yl)ethylamino)ethyl)amino)-3-oxopropyl(phenethyl)carbamate (0.065 g, 0.10 mmol) [Step iii] in a mixture of ethanol (10 mL) and 2 M hydrochloric acid (1 mL) was re-circulated through a 10% Pd/C cartridge using the H-Cube at 30° C. and 1 atm for 1 h. The solution was evaporated in vacuo and the residue purified by reverse phase HPLC (eluting with a gradient of acetonitrile in 0.2% aqueous trifluoroacetic acid) to afford the title compound as a white solid (10.00 mg).

MS (APCI+) 523 (M+H)⁺

¹H NMR (399.826 MHz, DMSO) δ 9.97-9.93 (m, 1H), 9.91-9.86 (m, 1H), 8.58-8.42 (m, 4H), 7.35 (m, 2H), 7.27 (m, 3H), 6.66 (m, 1H), 6.48 (m, 1H), 4.54 (s, 2H), 3.67 (m, 1H), 3.44 (m, 2H), 3.20 (m, 4H), 3.05 (m, 4H), 2.93 (m, 2H), 2.79 (m, 4H), 1.79-1.56 (m, 8H), 1.48 (m, 4H).

### EXAMPLE 25

### (R)—N-(Hexan-2-yl)-N-(2-(2-(5-hydroxy-3-oxo-3,4-dihydro-2H-benzo[b][1,4]oxazin-8-yl)ethylamino)ethyl)-3-(phenethylamino)propanamide bis(trifluoroacetate)

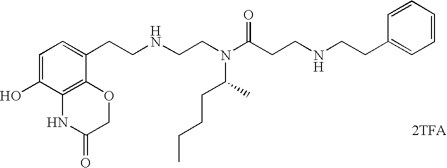

Prepared by an analogous procedure to Example 24, except using (R)-hexan-2-ylamine in place of heptylamine (in Step i) and 2-(3-chlorophenyl)ethylamine in place of 2-phenylethylamine (in Step ii).

MS (APCI+) 511 (M+H)⁺

¹H NMR (399.826 MHz, DMSO) δ 9.96-9.92 (m, 1H), 9.92-9.88 (m, 1H), 8.63-8.50 (m, 4H), 7.34 (m, 2H), 7.27 (m, 3H), 6.67 (m, 1H), 6.49 (m, 1H), 4.54 (s, 2H), 3.80 (m, 1H), 3.54-3.33 (m, 2H), 3.21 (m, 4H), 3.08 (m, 4H), 3.00 (m, 2H), 2.93 (m, 4H), 2.79 (m, 2H), 1.46 (m, 2H), 1.29 (m, 2H), 1.15 (d, J=5.9 Hz, 3H), 1.12 (t, J=9.1 Hz, 3H).

### EXAMPLE 26

### N-Cycloheptyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

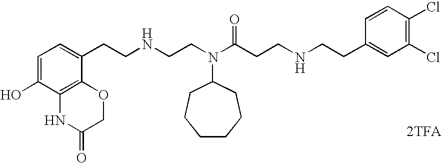

### Step i) Benzyl 2-(N-cycloheptyl-3-(3,4-dichlorophenethylamino)propanamido)ethyl(2-(5-hydroxy-3-oxo-3,4-dihydro-2H-benzo[b][1,4]oxazin-8-yl)ethyl)carbamate

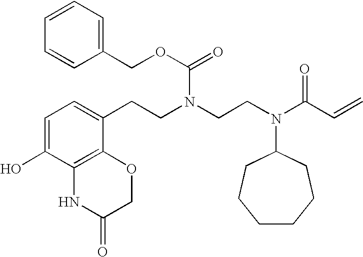

Prepared by an analogous procedure to Example 3, Steps i-iv, except using 8-(2-Aminoethyl)-5-hydroxy-2H-benzo[b][1,4]oxazin-3(4H)-one hydrochloride [Example 23, Step vii] in place of 5-(2-aminoethyl)-8-hydroxyquinolin-2(1H)-one hydrochloride.

### Step ii) Benzyl 2-(N-cycloheptyl-3-(3,4-dichlorophenethylamino)propanamido)ethyl(2-(5-hydroxy-3-oxo-3,4-dihydro-2H-benzo[b][1,4]oxazin-8-yl)ethyl)carbamate

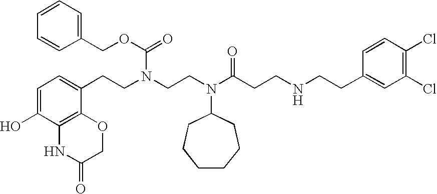

Benzyl 2-(N-cycloheptylacrylamido)ethyl(2-(5-hydroxy-3-oxo-3,4-dihydro-2H-benzo[b][1,4]oxazin-8-yl)ethyl)carbamate (0.1 g) [Step i] and 2-(3,4-dichlorophenyl)ethanamine (0.106 g) were stirred in ethanol (5 mL) at 60° C. for 20 h. The mixture was concentrated in vacuo and the residue purified by flash chromatography on silica (eluting with 10% methanol dichloromethane) to afford the subtitled compound as a light brown gum (0.065 g).

MS (ES+) 725 [M+H]⁺

### Step iii) N-cycloheptyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

A 1 M solution of boron tribromide in dichloromethane (0.5 mL) was added to a solution of benzyl 2-(N-cycloheptyl-3-(3,4-dichlorophenethylamino)propanamido)ethyl(2-(5-hydroxy-3-oxo-3,4-dihydro-2H-benzo[b][1,4]oxazin-8-yl)ethyl)carbamate (60 mg) [Step ii] in dichloromethane (2 mL) and the mixture was stirred for 1 h. Ice/water (˜1 mL) was added and the mixture evaporated in vacuo to remove the dichloromethane. The residue was diluted with acetonitrile and purified by reverse phase HPLC (eluting with acetonitrile in aqueous trifluoroacetic acid) to afford the title compound as a white solid (15.00 mg).

MS (APCI+) 591 [M+H]⁺

¹H NMR (399.826 MHz, DMSO) δ 9.98-9.93 (m, 1H), 9.90-9.83 (m, 1H), 8.52-8.37 (m, 4H), 7.61 (m, 2H), 7.30 (m, 1H), 6.67 (m, 1H), 6.49 (m, 1H), 4.54 (s, 2H), 3.68 (m, 1H), 3.45 (m, 2H), 3.25 (m, 2H), 3.16 (m, 2H), 3.11-2.99 (m, 4H), 2.95 (m, 2H), 2.79 (m, 4H), 1.80-1.39 (m, 12H).

### EXAMPLE 27

### 3-(3-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide bis(trifluoroacetate)

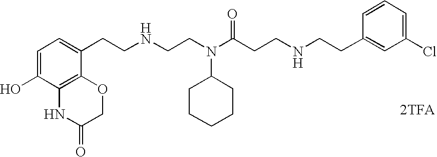

### Step i) N-Cyclohexyl-N-(2,2-dimethoxyethyl)acrylamide

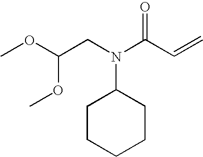

Triethylamine (4.47 mL) was added to a stirred solution of N-(2,2-dimethoxyethyl)cyclohexanamine (5.0 g) [Example 2, Step i] in dichloromethane (40 mL) was added. The mixture was cooled to 0° C. and a solution of acryloyl chloride (2.169 mL) in dichloromethane (10 mL) was added dropwise under nitrogen. The mixture was stirred at ambient temperature for 2 h and then washed with water (×2), saturated aqueous sodium hydrogen carbonate, and water again. The organic phase was dried over anhydrous sodium sulfate, filtered and concentrated in vacuo to give the subtitled compound as an oil (6.39 g, 99%).

¹H NMR (299.947 MHz, DMSO) δ 6.86-6.68 (m, 1H), 6.07 (d, J=16.9 Hz, 1H), 5.68-5.56 (m, 1H), 4.53-4.33 (m, 1H), 4.08-3.64 (m, 1H), 3.42-3.26 (m, 8H), 1.79-0.99 (m, 10H).

### Step ii) N-Cyclohexyl-N-(2-oxoethyl)acrylamide

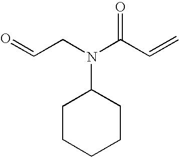

Prepared by analogous procedure to Example 19, Step v, using N-cyclohexyl-N-(2,2-dimethoxyethyl)acrylamide [Step i] in place of benzyl 3-(cyclohexyl(2,2-dimethoxyethyl)amino)-3-oxopropyl(3-fluorophenethyl)carbamate.

MS (ES+) 196 [M+H]⁺

### Step iii) tert-Butyl 2-(N-cyclohexylacrylamido)ethyl(2-(5-hydroxy-3-oxo-3,4-dihydro-2H-benzo[b][1,4]oxazin-8-yl)ethyl)carbamate

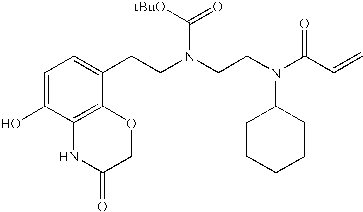

8-(2-Aminoethyl)-5-hydroxy-2H-benzo[b][1,4]oxazin-3(4H)-one hydrochloride (2.1 g) [Example 23, Step vii] and N-cyclohexyl-N-(2-oxoethyl)acrylamide (2.0 g) [Step ii] were dissolved in a mixture of NMP (20 mL) and water (2 mL), Sodium hydrogen carbonate (0.793 g) was added and the reaction stirred for 15 min. Sodium triacetoxyborohydride (2.73 g) was added and stirring continued for a further 20 h. The mixture was diluted with ethyl acetate (50 mL) and a solution of sodium hydrogen carbonate (3.61 g) in water (50 mL), and di-tert-butyl dicarbonate (2.248 g) was added. After stirring for 2 h, the mixture was diluted with ethyl acetate (100 mL) and washed with water (3×50 mL) and brine. The organic phase was dried over anhydrous sodium sulfate and evaporated in vacuo. The residue was purified by flash chromatography on silica (eluting with 70% ethyl acetate isohexane to 10% methanol in dichloromethane) to afford the subtitled compound as a white solid (0.870 g).

MS (APCI+) 488 [M+H]⁺

### Step iv) 3-(3-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide bis(trifluoroacetate)

tert-Butyl 2-(N-cyclohexylacrylamido)ethyl(2-(5-hydroxy-3-oxo-3,4-dihydro-2H-benzo[b][1,4]oxazin-8-yl)ethyl)carbamate (0.1 g) [Step iii] and 2-(3-chlorophenyl)ethanamine (0.820 mL, 0.41 mmol) were dissolved in ethanol (1 mL) and heated at 50° C. for 20 h. The solvent was evaporated in vacuo and the residue dissolved in dichloromethane (1 mL). Trifluoroacetic acid (1 mL, 12.98 mmol) was added and the mixture stirred for 2 h, then concentrated in vacuo. The residue was purified by reverse phase HPLC with (eluting with acetonitrile in 0.2% aqueous trifluoroacetic acid) to afford the title compound as a white solid (0.125 g).

MS (APCI+) 543 [M+H]⁺

¹H NMR (399.826 MHz, DMSO) δ 9.94 (s, 1H), 9.89 (s, 1H), 8.65-8.48 (m, 4H), 7.41-7.31 (m, 3H), 7.25 (m, 1H), 6.66 (m, 1H), 6.49 (m, 1H), 4.54 (s, 2H), 3.59-3.31 (m, 3H), 3.22 (m, 4H), 3.07 (m, 2H), 2.97 (m, 4H), 2.80 (m, 4H), 1.78 (m, 2H), 1.68 (m, 2H), 1.62 (m, 2H), 1.46 (m, 2H), 1.33 (m, 2H).

### EXAMPLE 28

### 3-(2-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide bis(trifluoroacetate)

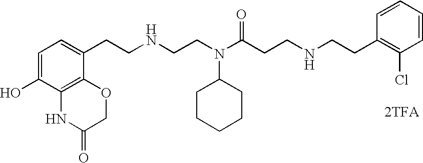

Prepared by an analogous procedure to Example 27, Step iv, using 2-(2-chlorophenyl)ethanamine in place of 2-(3-chlorophenyl)ethanamine.

MS (APCI+) 543 [M+H]⁺

¹H NMR (399.826 MHz, DMSO) δ 9.93 (s, 2H), 8.91-8.73 (m, 2H), 8.69-8.57 (m, 2H), 7.47 (m, 1H), 7.40 (m, 1H), 7.33 (m, 2H), 6.66 (m, 1H), 6.49 (m, 1H), 4.53 (s, 2H), 3.68-3.43 (m, 3H), 3.21 (m, 4H), 3.08 (m, 4H), 2.99 (m, 2H), 2.81 (m, 4H), 1.78 (m, 2H), 1.69 (m, 2H), 1.61 (m, 2H), 1.46 (m, 2H), 1.33 (m, 2H).

### EXAMPLE 29

### N-Cyclohexyl-3-(2,3-dichlorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

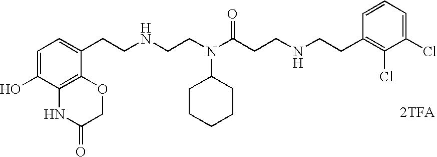

Prepared by an analogous procedure to Example 27, Step iv, using 2-(3,4-dichlorophenyl)ethanamine in place of 2-(3-chlorophenyl)ethanamine.

MS (APCI+) 577 [M+H]⁺

¹H NMR (399.826 MHz, DMSO) δ 9.93 (s, 1H), 9.89 (s, 1H), 8.81-8.64 (m, 2H), 8.61-8.49 (m, 2H), 7.59 (m, 1H), 7.38 (m, 2H), 6.66 (m, 1H), 6.49 (m, 1H), 4.53 (s, 2H), 3.59-3.31 (m, 5H), 3.22 (m, 4H), 3.14 (m, 2H), 3.06 (m, 2H), 2.98 (m, 2H), 2.80 (m, 4H), 1.78 (m, 2H), 1.69 (m, 2H), 1.59 (m, 2H), 1.46 (m, 2H), 1.32 (m, 2H).

### EXAMPLEs 30-42

Prepared by analogous procedures to Example 27, Step iv, with appropriate substitution of reagents.

### EXAMPLE 30

### N-Cyclohexyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

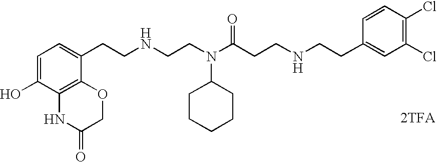

¹H NMR (399.826 MHz, DMSO) δ 9.97-9.89 (m, 2H), 8.83-8.51 (m, 4H), 7.61 (m, 2H), 7.30 (m, 1H), 6.66 (m, 1H), 6.49 (m, 1H), 4.54 (s, 2H), 3.58-3.34 (m, 3H), 3.29-3.21 (m, 2H), 3.20-3.13 (m, 2H), 3.11-3.03 (m, 2H), 3.01-2.92 (m, 4H), 2.84-2.75 (m, 4H), 1.82-1.73 (m, 2H), 1.72-1.53 (m, 3H), 1.52-1.40 (m, 2H), 1.38-1.22 (m, 2H), 1.14-1.01 (m, 1H).

### EXAMPLE 31

### N-Cyclohexyl-3-(2,4-dichlorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

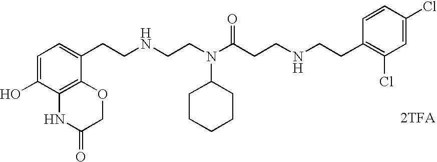

¹H NMR (399.826 MHz, DMSO) δ 9.94 (s, 1H), 9.87 (s, 1H), 8.70-8.40 (m, 4H), 7.66 (m, 1H), 7.45 (m, 2H), 6.66 (m, 1H), 6.48 (m, 1H), 4.54 (s, 2H), 3.60-3.27 (m, 3H), 3.20 (m, 4H), 3.06 (m, 4H), 2.98 (m, 2H), 2.84-2.74 (m, 4H), 1.82-1.74 (m, 2H), 1.72-1.59 (m, 3H), 1.52-1.40 (m, 2H), 1.39-1.21 (m, 2H), 1.15-1.02 (m, 1H).

### EXAMPLE 32

### 3-(4-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide bis(trifluoroacetate)

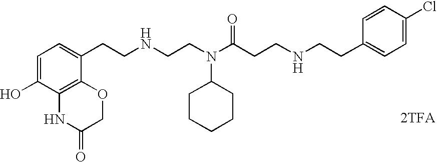

¹H NMR (399.826 MHz, DMSO) δ 9.96-9.89 (m, 2H), 8.82-8.50 (m, 4H), 7.41 (m, 2H), 7.31 (m, 2H), 6.66 (m, 1H), 6.49 (m, 1H), 4.54 (s, 2H), 3.58-3.33 (m, 3H), 3.26-3.13 (m, 4H), 3.12-3.02 (m, 2H), 3.01-2.90 (m, 4H), 2.84-2.75 (m, 4H), 1.82-1.73 (m, 2H), 1.72-1.56 (m, 3H), 1.55-1.39 (m, 2H), 1.39-1.22 (m, 2H), 1.15-1.01 (m, 1H).

### EXAMPLE 33

### N-Cyclohexyl-3-(2-fluoro-5-methylphenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

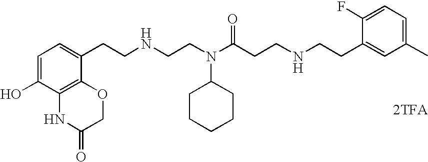

¹H NMR (399.825 MHz, CD₃OD) δ 7.10 (m, 2H), 6.96 (m, 1H), 6.71 (d, J=8.6 Hz, 1H), 6.47 (d, J=8.7 Hz, 1H), 4.59 (s, 2H), 3.60 (m, 3H), 3.31 (m, 4H), 3.17 (t, J=7.4 Hz, 2H), 3.11 (t, J=5.7 Hz, 2H), 3.02 (t, J=8.0 Hz, 2H), 2.89 (m, 4H), 2.28 (s, 3H), 1.89-1.62 (m, 5H), 1.55-1.09 (m, 5H).

### EXAMPLE 34

### 3-(5-Chloro-2-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide bis(trifluoroacetate)

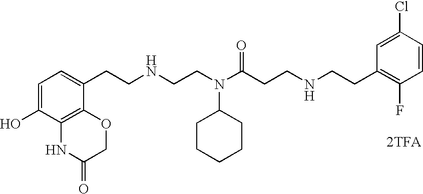

¹H NMR (399.825 MHz, CD₃OD) δ 7.38 (m, 1H), 7.29 (m, 1H), 7.11 (m, 1H), 6.71 (d, J=8.7 Hz, 1H), 6.47 (d, J=8.7 Hz, 1H), 4.60 (s, 2H), 3.59 (m, 3H), 3.32 (m, 4H), 3.17 (t, J=7.3 Hz, 2H), 3.12 (t, J=5.7 Hz, 2H), 3.06 (t, J=7.8 Hz, 2H), 2.90 (m, 4H), 1.88-1.61 (m, 5H), 1.55-1.08 (m, 5H).

### EXAMPLE 35

### N-Cyclohexyl-3-(3,5-difluorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

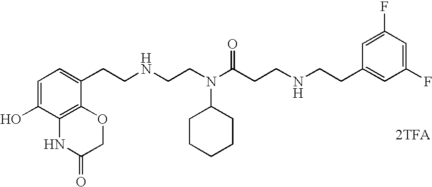

¹H NMR (399.825 MHz, CD₃OD) δ 6.94 (m, 2H), 6.84 (m, 1H), 6.71 (d, J=9.0 Hz, 1H), 6.47 (d, J=9.0 Hz, 1H), 4.60 (s, 2H), 3.58 (m, 3H), 3.32 (m, 4H), 3.17 (t, J=7.5 Hz, 2H), 3.11 (t, J=6.0 Hz, 2H), 3.04 (t, J=7.9 Hz, 2H), 2.89 (m, 4H), 1.88-1.61 (m, 5H), 1.55-1.08 (m, 5H).

### EXAMPLE 36

### N-Cyclohexyl-3-(2,3-difluorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

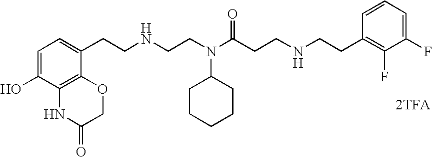

¹H NMR (399.825 MHz, CD₃OD) δ 7.22-7.06 (m, 3H), 6.71 (d, J=8.2 Hz, 1H), 6.47 (d, J=8.2 Hz, 1H), 4.60 (s, 2H), 3.59 (m, 3H), 3.32 (m, 4H), 3.17 (t, J=7.4 Hz, 2H), 3.11 (m, 4H), 2.89 (m, 4H), 1.88-1.62 (m, 5H), 1.55-1.09 (m, 5H).

### EXAMPLE 37

### 3-(3-Chloro-4-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide bis(trifluoroacetate)

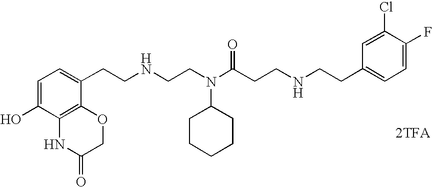

¹H NMR (399.825 MHz, CD₃OD) δ 7.44 (m, 1H), 7.25 (m, 1H), 7.19 (m, 1H), 6.71 (d, J=9.2 Hz, 1H), 6.47 (d, J=9.2 Hz, 1H), 4.60 (s, 2H), 3.58 (m, 3H), 3.31 (m, 4H), 3.17 (t, J=7.5 Hz, 2H), 3.11 (t, J=5.7 Hz, 2H), 3.00 (t, J=8.1 Hz, 2H), 2.89 (m, 4H), 1.87-1.61 (m, 5H), 1.55-1.08 (m, 5H).

### EXAMPLE 38

### N-Cyclohexyl-3-(2,5-difluorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

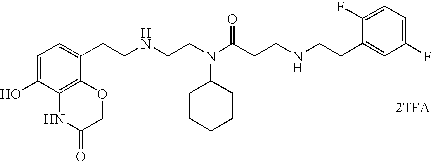

¹H NMR (399.825 MHz, CD₃OD) δ 7.13 (m, 2H), 7.04 (m, 1H), 6.71 (d, J=9.0 Hz, 1H), 6.47 (d, J=9.0 Hz, 1H), 4.60 (s, 2H), 3.59 (m, 3H), 3.32 (m, 4H), 3.17 (t, J=7.6 Hz, 2H), 3.12 (t, J=6.2 Hz, 2H), 3.06 (t, J=7.9 Hz, 2H), 2.89 (m, 4H), 1.89-1.62 (m, 5H), 1.55-1.09 (m, 5H).

### EXAMPLE 39

### 3-(3-Chloro-2-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide bis(trifluoroacetate)

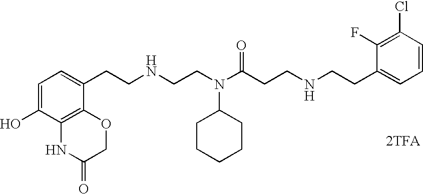

¹H NMR (399.825 MHz, CD₃OD) δ 7.40 (m, 1H), 7.29 (m, 1H), 7.14 (m, 1H), 6.71 (m, 1H), 6.47 (m, 1H), 4.60 (s, 2H), 3.59 (m, 3H), 3.32 (m, 4H), 3.17 (m, 2H), 3.11 (m, 4H), 2.89 (m, 4H), 1.88-1.10 (m, 10H).

### EXAMPLE 40

### N-Cyclohexyl-3-(4-fluoro-3-methylphenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

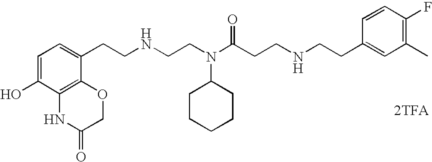

¹H NMR (399.825 MHz, CD₃OD) δ 7.14 (m, 1H), 7.08 (m, 1H), 6.98 (m, 1H), 6.70 (d, J=8.3 Hz, 1H), 6.47 (d, J=8.3 Hz, 1H), 4.55 (s, 2H), 3.60 (t, J=6.3 Hz, 2H), 3.51 (t, J=7.2 Hz, 2H), 3.30 (m, 1H), 3.21 (m, 4H), 3.13 (t, J=6.4 Hz, 2H), 2.93 (t, J=7.9 Hz, 2H), 2.82 (t, J=7.0 Hz, 2H), 2.71 (t, J=6.2 Hz, 2H), 2.23 (s, 3H), 2.07-2.01 (m, 2H), 1.89-1.82 (m, 2H), 1.73-1.65 (m, 1H), 1.40-1.13 (m, 5H).

### EXAMPLE 41

### 3-(3-Chloro-5-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide (trifluoroacetate)

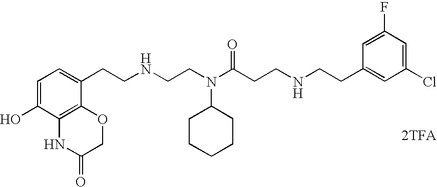

¹H NMR (399.825 MHz, CD₃OD) δ 7.13 (m, 1H), 7.04 (m, 1H), 6.99 (m, 1H), 6.65 m, 1H), 6.42 (m, 1H), 4.55 (s, 2H), 3.55 (m, 1H), 3.52 (t, J=6.1 Hz, 2H), 3.29-3.21 (m, 4H), 3.11 (t, J=7.6 Hz, 2H), 3.05 (t, J=6.1 Hz, 2H), 2.96 (t, J=7.8 Hz, 2H), 2.87-2.79 (m, 4H), 1.82-1.74 (m, 2H), 1.73-1.65 (m, 2H), 1.64-1.55 (m, 1H), 1.50-1.19 (m, 4H), 1.16-1.02 (m, 1H).

### EXAMPLE 42

### N-Cyclohexyl-3-(3,4-difluorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

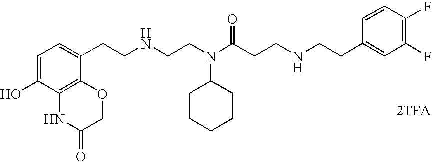

¹H NMR (399.825 MHz, CD₃OD) δ 7.16 (m, 2H), 7.03 (m, 1H), 6.66 (m, 1H), 6.43 (m, 1H), 4.54 (s, 2H), 3.55 (m, 1H), 3.51 (t, J=5.8 Hz, 2H), 3.27-3.20 (m, 4H), 3.10 (t, J=7.5 Hz, 2H), 3.05 (t, J=5.8 Hz, 2H), 2.94 (t, J=7.5 Hz, 2H), 2.86-2.80 (m, 4H), 1.77 (m, 2H), 1.68 (m, 2H), 1.59 (m, 1H), 1.42 (m, 2H), 1.31 (m, 2H), 1.09 (m, 1H).

### EXAMPLE 43

### 4-(2-(3-(Cyclohexyl(2-(2-(5-hydroxy-3-oxo-3,4-dihydro-2H-benzo[b][1,4]oxazin-8-yl)ethylamino)ethyl)amino)-3-oxopropylamino)ethyl)benzoic acid bis(trifluoroacetate)

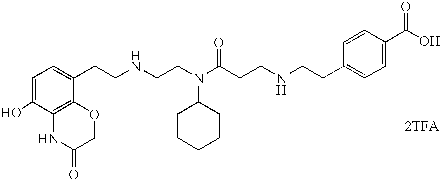

Prepared by an analogous procedure to Example 27, Step iv, using methyl 4-(2-aminoethyl)benzoate hydrochloride and triethylamine, in place of 2-(3-chlorophenyl)ethanamine. Saponification was achieved by treatment of an methanolic solution of the initially formed methyl ester with sodium hydroxide, prior to standard cleavage of the tert-butyl carbamoyl group.

¹H NMR (399.826 MHz, DMSO) δ 9.93 (s, 1H), 7.92 (d, J=8.0 Hz, 2H), 7.41 (d, J=8.0 Hz, 2H), 6.66 (m, 1H), 6.49 (m, 1H), 4.54 (s, 2H), 3.70-3.22 (m, 5H), 3.19 (t, J=6.8 Hz, 2H), 3.03 (m, 6H), 2.80 (m, 4H), 1.81-1.01 (m, 10H).

### EXAMPLE 44

### (R)-3-(3-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

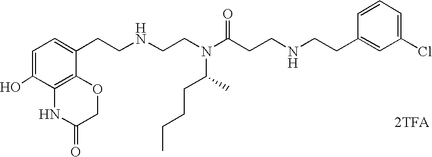

### Step i) (R)—N-(2,2-Dimethoxyethyl)hexan-2-amine

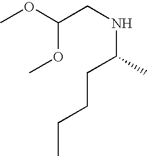

Prepared by an analogous procedure to Example 2, Step ii, using (R)-hexan-2-amine in place of cyclohexylamine.

¹H NMR (399.824 MHz, CDCl₃) δ 4.47 (t, J=5.5 Hz, 1H), 3.39 (s, 6H), 2.76 (dd, J=11.8, 5.4 Hz, 1H), 2.69 (dd, J=11.9, 5.8 Hz, 1H), 2.60 (q, J=5.7 Hz, 1H), 1.50-1.40 (m, 2H), 1.34-1.25 (m, 5H), 1.04 (d, J=6.2 Hz, 3H), 0.90 (t, J=6.4 Hz, 3H).

### Step ii) (R)—N-(2,2-Dimethoxyethyl)-N-(hexan-2-yl)acrylamide

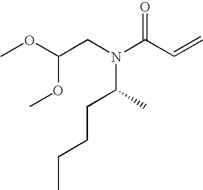

Prepared by an analogous procedure to Example 27, Step i, using (R)—N-(2,2-dimethoxyethyl)hexan-2-amine [Step i] in place of N-(2,2-dimethoxyethyl)cyclohexanamine.

¹H NMR (399.824 MHz, CDCl₃) δ 6.78-6.56 (m, 1H), 6.35-6.23 (m, 1H), 5.67-5.61 (m, 1H), 4.69 (t, J=5.0 Hz, 1H), 4.49-4.37 (m, 1H), 3.97-3.86 (m, 1H), 3.47-3.19 (m, 7H), 1.64-1.11 (m, 9H), 0.88 (t, J=7.0 Hz, 3H).

### Step iii) (R)-tert-Butyl 2-(5-(tert-butoxycarbonyloxy)-3-oxo-3,4-dihydro-2H-benzo[b][1,4]oxazin-8-yl)ethyl(2-(N-(hexan-2-yl)-3-methoxypropanamido)ethyl)carbamate

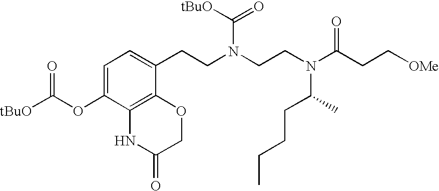

p-Toluenesulfonic acid (2.78 g) was added to a stirred solution of (R)—N-(2,2-dimethoxyethyl)-N-(hexan-2-yl)acrylamide (3.56 g) [Step ii] in dichloromethane (60 mL) and the mixture stirred at ambient temperature for 4 h. The solution was then washed sequentially with saturated sodium bicarbonate solution (×2) and brine, dried over sodium sulfate, filtered and the solvent removed in vacuo. The residue was dissolved in NMP (30 mL) and water (3 mL) and 8-(2-aminoethyl)-5-hydroxy-2H-benzo[b][1,4]oxazin-3(4H)-one, hydrochloride (2.39 g) [Example 23, Step vii] was added, followed by sodium bicarbonate (0.903 g). The mixture was stirred at ambient temperature for 15 mins, sodium triacetoxyborohydride (3.11 g) was added and the reaction mixture stirred at ambient temperature for 18 h. The mixture was diluted with water, acidified with 1 N aqueous hydrochloric acid, stirred for 5 minutes, then basified with sodium bicarbonate solution and extracted into ethyl acetate (×3). The organic layer was dried over magnesium sulfate, filtered and concentrated in vacuo. The aqueous layer was concentrated in vacuo, ethyl acetate was added and the resulting mixture dried over magnesium sulfate, filtered and concentrated in vacuo. Both product containing portions were combined, dissolved in dichloromethane (50 mL) and the solution treated with triethylamine (2.72 mL) and di-tert-butyl dicarbonate (3.40 mL). The mixture was stirred at room temperature for 18 hours, then diluted with saturated sodium bicarbonate solution and extracted into dichloromethane (×3). The combined extracts were washed sequentially with saturated sodium bicarbonate solution and brine, dried over sodium sulfate, filtered and concentrated in vacuo. The residue was purified by flash chromatography on silica (eluting with a 10-100% gradient of ethyl acetate in isohexane) to afford the sub-titled compound as a white solid (1.83 g).

MS (APCI+) 622 [M+H]⁺

¹H NMR (399.824 MHz, CDCl₃) δ 7.78-7.66 (m, 1H), 6.92-6.75 (m, 2H), 4.76-4.53 (m, 2H), 3.75-3.65 (m, 2H), 3.50-3.10 (m, 9H), 2.90-2.53 (m, 5H), 1.56 (d, J=2.1 Hz, 9H), 1.50-1.09 (m, 18H), 0.93-0.85 (m, 3H).

### Step iv) (R)-tert-Butyl 2-(N-(hexan-2-yl)acrylamido)ethyl(2-(5-hydroxy-3-oxo-3,4-dihydro-2H-benzo[b][1,4]oxazin-8-yl)ethyl)carbamate

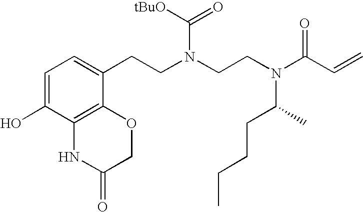

Lithium tert-butoxide (1.178 g) was added to a solution of (R)-tert-butyl 2-(5-(tert-butoxycarbonyloxy)-3-oxo-3,4-dihydro-2H-benzo[b][1,4]oxazin-8-yl)ethyl(2-(N-(hexan-2-yl)-3-methoxypropanamido)ethyl)carbamate (1.83 g) [Step iii] in DMF (20 mL) and the mixture stirred at ambient temperature under nitrogen for 2 h. Water was added and the mixture was acidified with 2 N hydrochloric acid to pH4 and extracted with ethyl acetate (×3). The combined extracts were washed with water (×3) and brine, dried over sodium sulfate, filtered and concentrated in vacuo to afford the sub-titled compound as an orange oil (1.440 g).

MS (APCI+) 490 [M+H]⁺

### Step v) (R)-3-(3-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoro acetate)

Prepared by an analogous procedure to Example 27, Step iv, using (R)-tert-Butyl 2-(N-(hexan-2-yl)acrylamido)ethyl(2-(5-hydroxy-3-oxo-3,4-dihydro-2H-benzo[b][1,4]oxazin-8-yl)ethyl)carbamate [Step iv] in place of tert-Butyl 2-(N-cyclohexylacrylamido)ethyl(2-(5-hydroxy-3-oxo-3,4-dihydro-2H-benzo[b][1,4]oxazin-8-yl)ethyl)carbamate.

MS (APCI+) 545 [M+H]⁺

¹H NMR (399.825 MHz, CD₃OD) δ 7.35-7.21 (m, 4H), 6.72 (d, J=8.5 Hz, 1H), 6.48 (d, J=8.5 Hz, 1H), 4.60 (s, 2H), 3.91-3.81 (m, 1H), 3.64-3.55 (m, 1H), 3.49-3.41 (m, 1H), 3.34-3.26 (m, 4H), 3.21-3.12 (m, 4H), 3.01 (t, S=8.9 Hz, 2H), 2.94-2.78 (m, 4H), 1.53 (q, J=8.1 Hz, 2H), 1.38-1.15 (m, 7H), 0.91 (t, J=7.2 Hz, 3H).

### EXAMPLEs 45-57

Prepared by analogous procedures to Example 44, Step v, with appropriate substitution of reagents.

### EXAMPLE 45

### (R)-3-(4-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

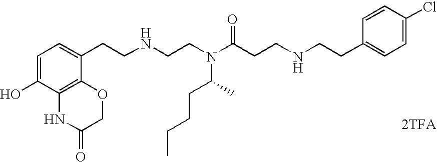

¹H NMR (399.825 MHz, CD₃OD) δ 7.35-7.24 (m, 4H), 6.71 (d, J=8.2 Hz, 1H), 6.48 (d, J=8.2 Hz, 1H), 4.60 (s, 2H), 3.91-3.80 (m, 1H), 3.64-3.55 (m, 1H), 3.49-3.41 (m, 1H), 3.33-3.28 (m, 4H), 3.21-3.11 (m, 4H), 3.03-2.97 (m, 2H), 2.94-2.77 (m, 4H), 1.56-1.49 (m, 2H), 1.39-1.14 (m, 7H), 0.91 (t, J=7.2 Hz, 3H).

### EXAMPLE 46

### (R)-3-(2-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

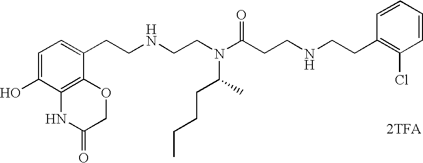

¹H NMR (399.825 MHz, CD₃OD) δ 7.43-7.35 (m, 2H), 7.31-7.24 (m, 2H), 6.71 (d, J=8.5 Hz, 1H), 6.47 (d, J=8.5 Hz, 1H), 4.59 (s, 2H), 3.92-3.82 (m, 1H), 3.69-3.57 (m, 1H), 3.50-3.42 (m, 1H), 3.36-3.28 (m, 4H), 3.22-3.11 (m, 6H), 2.95-2.79 (m, 4H), 1.57-1.49 (m, 2H), 1.39-1.13 (m, 7H), 0.91 (t, J=7.2 Hz, 3H).

### EXAMPLE 47

### (R)-3-(4-Fluoro-3-methylphenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

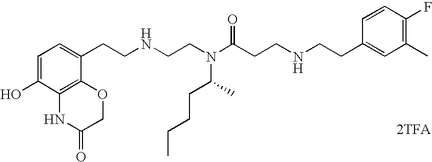

¹H NMR (399.825 MHz, CD₃OD) δ 7.17-7.06 (m, 2H), 7.00-6.94 (m, 1H), 6.71 (d, J=8.5 Hz, 1H), 6.48 (d, J=8.2 Hz, 1H), 4.60 (s, 2H), 3.91-3.81 (m, 1H), 3.64-3.56 (m, 1H), 3.49-3.41 (m, 1H), 3.32-3.25 (m, 4H), 3.21-3.11 (m, 4H), 2.98-2.77 (m, 6H), 2.22 (d, J=1.8 Hz, 3H), 1.56-1.49 (m, 2H), 1.39-1.14 (m, 7H), 0.91 (t, J=7.2 Hz, 3H).

### EXAMPLE 48

### (R)-3-(3-Chloro-4-fluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

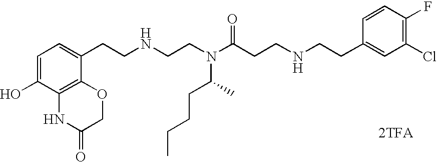

¹H NMR (399.825 MHz, CD₃OD) δ 7.46-7.42 (m, 1H), 7.27-7.16 (m, 2H), 6.71 (d, J=8.5 Hz, 1H), 6.48 (d, J=8.5 Hz, 1H), 4.60 (s, 2H), 3.91-3.80 (m, 1H), 3.64-3.56 (m, 1H), 3.49-3.41 (m, 1H), 3.34-3.28 (m, 4H), 3.21-3.11 (m, 4H), 3.03-2.97 (m, 2H), 2.94-2.78 (m, 4H), 1.56-1.49 (m, 2H), 1.39-1.14 (m, 7H), 0.91 (t, J=7.2 Hz, 3H).

### EXAMPLE 49

### (R)-3-(2-(5-Chlorothiophen-2-yl)ethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

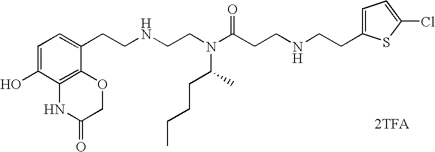

¹H NMR (399.825 MHz, CD₃OD) δ 6.84-6.79 (m, 2H), 6.71 (d, J=8.2 Hz, 1H), 6.48 (d, J=8.2 Hz, 1H), 4.60 (s, 2H), 3.90-3.81 (m, 1H), 3.64-3.56 (m, 1H), 3.50-3.41 (m, 1H), 3.36-3.28 (m, 4H), 3.21-3.11 (m, 6H), 2.94-2.78 (m, 4H), 1.56-1.49 (m, 2H), 1.39-1.14 (m, 7H), 0.91 (t, J=7.2 Hz, 3H).

### EXAMPLE 50

### (R)-3-(3-Chloro-5-fluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

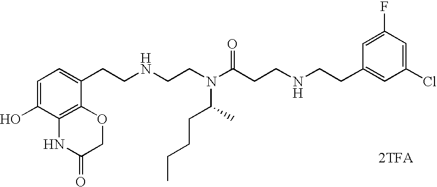

¹H NMR (399.825 MHz, CD₃OD) δ 7.20-7.18 (m, 1H), 7.12-7.03 (m, 2H), 6.71 (d, J=8.2 Hz, 1H), 6.48 (d, J=8.2 Hz, 1H), 4.60 (s, 2H), 3.91-3.81 (m, 1H), 3.64-3.55 (m, 1H), 3.49-3.41 (m, 1H), 3.36-3.27 (m, 4H), 3.21-3.12 (m, 4H), 3.06-3.00 (m, 2H), 2.94-2.78 (m, 4H), 1.57-1.49 (m, 2H), 1.39-1.14 (m, 7H), 0.91 (t, J=7.2 Hz, 3H).

### EXAMPLE 51

### (R)-3-(3,5-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

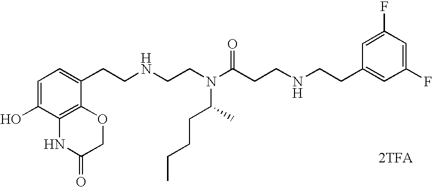

¹H NMR (399.825 MHz, CD₃OD) δ 6.97-6.91 (m, 2H), 6.88-6.81 (m, 1H), 6.71 (d, J=8.5 Hz, 1H), 6.48 (d, J=8.5 Hz, 1H), 4.60 (s, 2H), 3.91-3.81 (m, 1H), 3.64-3.55 (m, 1H), 3.49-3.41 (m, 1H), 3.37-3.26 (m, 4H), 3.21-3.12 (m, 4H), 3.07-3.01 (m, 2H), 2.93-2.80 (m, 4H), 1.57-1.49 (m, 2H), 1.39-1.15 (m, 7H), 0.91 (t, J=7.2 Hz, 3H).

### EXAMPLE 52

### (R)-3-(3,4-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

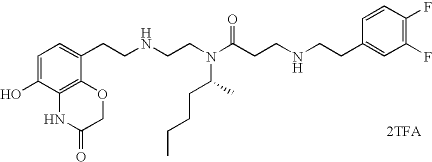

¹H NMR (399.825 MHz, CD₃OD) δ 7.27-7.17 (m, 2H), 7.11-7.06 (m, 1H), 6.71 (d, J=8.5 Hz, 1H), 6.48 (d, J=8.5 Hz, 1H), 4.60 (s, 2H), 3.91-3.81 (m, 1H), 3.64-3.55 (m, 1H), 3.50-3.40 (m, 1H), 3.34-3.27 (m, 4H), 3.21-3.10 (m, 4H), 3.03-2.97 (m, 2H), 2.94-2.77 (m, 4H), 1.56-1.48 (m, 2H), 1.39-1.13 (m, 7H), 0.91 (t, J=7.2 Hz, 3H).

### EXAMPLE 53

### (R)-3-(2,3-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

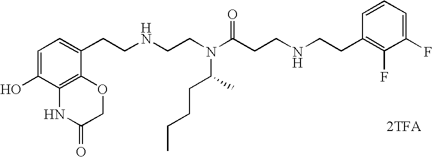

¹H NMR (399.825 MHz, CD₃OD) δ 7.23-7.10 (m, 3H), 6.71 (d, J=8.2 Hz, 1H), 6.48 (d, J=8.5 Hz, 1H), 4.60 (s, 2H), 3.92-3.82 (m, 1H), 3.64-3.56 (m, 1H), 3.50-3.41 (m, 1H), 3.36-3.28 (m, 4H), 3.22-3.09 (m, 6H), 2.94-2.81 (m, 4H), 1.57-1.48 (m, 2H), 1.39-1.15 (m, 7H), 0.91 (t, J=7.2 Hz, 3H).

### EXAMPLE 54

### (R)-3-(2,5-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

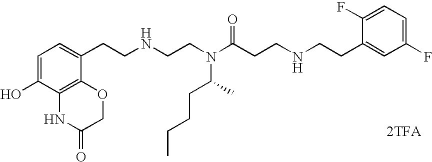

¹H NMR (399.825 MHz, CD₃OD) δ 7.16-7.09 (m, 2H), 7.07-7.00 (m, 1H), 6.71 (d, J=8.2 Hz, 1H), 6.47 (d, J=8.2 Hz, 1H), 4.60 (s, 2H), 3.91-3.81 (m, 1H), 3.64-3.56 (m, 1H), 3.49-3.41 (m, 1H), 3.35-3.27 (m, 4H), 3.21-3.12 (m, 4H), 3.10-3.04 (m, 2H), 2.95-2.80 (m, 4H), 1.57-1.48 (m, 2H), 1.40-1.15 (m, 7H), 0.91 (t, J=7.2 Hz, 3H).

### EXAMPLE 55

### (R)-3-(3,4-Dichlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

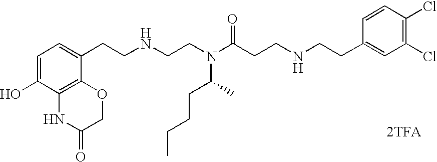

¹H NMR (399.825 MHz, CD₃OD) δ 7.50-7.46 (m, 2H), 7.25-7.21 (m, 1H), 6.71 (d, J=9.2 Hz, 1H), 6.48 (d, J=8.5 Hz, 1H), 4.60 (s, 2H), 3.91-3.81 (m, 1H), 3.64-3.55 (m, 1H), 3.49-3.41 (m, 1H), 3.35-3.26 (m, 4H), 3.21-3.11 (m, 4H), 3.04-2.98 (m, 2H), 2.93-2.78 (m, 4H), 1.57-1.49 (m, 2H), 1.39-1.14 (m, 7H), 0.91 (t, J=7.2 Hz, 3H).

### EXAMPLE 56

### (R)-3-(2-Fluoro-5-methylphenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

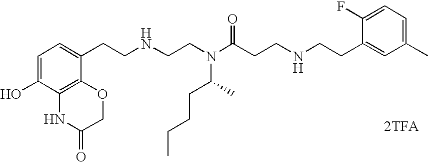

¹H NMR (399.825 MHz, CD₃OD) δ 7.14-7.06 (m, 2H), 7.00-6.93 (m, 1H), 6.71 (d, J=8.2 Hz, 1H), 6.48 (d, J=8.5 Hz, 1H), 4.59 (s, 2H), 3.91-3.81 (m, 1H), 3.65-3.56 (m, 1H), 3.49-3.41 (m, 1H), 3.34-3.26 (m, 4H), 3.21-3.12 (m, 4H), 3.06-2.99 (m, 2H), 2.94-2.78 (m, 4H), 2.28 (s, 3H), 1.57-1.48 (m, 2H), 1.40-1.14 (m, 7H), 0.91 (t, J=7.2 Hz, 3H).

### EXAMPLE 57

### (R)-3-(5-Fluoro-2-methylphenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide bis(trifluoroacetate)

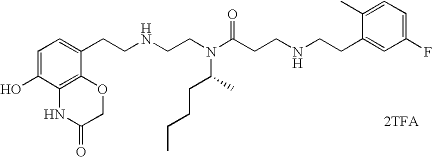

¹H NMR (399.825 MHz, CD₃OD) δ 7.21-7.15 (m, 1H), 7.00-6.96 (m, 1H), 6.91-6.85 (m, 1H), 6.71 (d, J=8.2 Hz, 1H), 6.47 (d, J=8.2 Hz, 1H), 4.59 (s, 2H), 3.92-3.82 (m, 1H), 3.65-3.57 (m, 1H), 3.50-3.42 (m, 1H), 3.35-3.23 (m, 4H), 3.21-3.13 (m, 4H), 3.05-2.98 (m, 2H), 2.94-2.83 (m, 4H), 2.31 (s, 3H), 1.57-1.49 (m, 2H), 1.39-1.15 (m, 7H), 0.91 (t, J=7.2 Hz, 3H).

### EXAMPLE 58-59

Prepared by an analogous procedure to Example 24, Steps i-iii, with appropriate substitution of reagents. Final deprotection following the procedure of Example 26, Step iii.

### EXAMPLE 58

### (R)—N-sec-Butyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide dihydrochloride

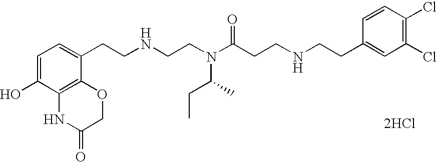

### Step i) (R)-benzyl 3-(sec-butyl(2-oxoethyl)amino)-3-oxopropyl(3,4-dichlorophenethyl)carbamate

Prepared by an analogous procedure to Example 24, Steps i-ii, with appropriate substitution of reagents.

### Step ii) (R)—N-sec-Butyl-3-(3,4-dichlorophenethylamino)-N-2-(2-(5-hydroxy-3-oxo-3,4-dihydro-2H-benzo[b][1,4]oxazin-8-yl)ethylamino)ethyl)propanamide dihydrochloride

A solution of (R)-benzyl 3-(sec-butyl(2-oxoethyl)amino)-3-oxopropyl(3,4-dichlorophenethyl)carbamate (403 mg) [Step i] in NMP (1.64 mL) and triethylamine (0.057 mL, 0.41 mmol) were added to a solution of 8-(2-aminoethyl)-5-hydroxy-2H-benzo[b][1,4]oxazin-3(4H)-one, hydrochloride (100 mg) [Example 23, Step vii] in NMP (5 mL) and the mixture stirred at ambient temperature for 15 mins. sodium triacetoxyborohydride (173 mg, 0.82 mmol) was added and the mixture stirred for 4 then diluted with water and extracted into ethyl acetate (×3). The combined extracts were washed with brine, dried over sodium sulfate, filtered and concentrated in vacuo. The residue was dissolved in dichloromethane (2 mL) and a 1 M solution of boron tribromide in dichloromethane (1 mL) was added. The mixture was stirred at ambient temperature for 3 h, then quenched with ice/water. The dichloromethane was removed by concentration in vacuo and the residue was diluted with methanol, filtered and purified by preparative HPLC (eluting with a gradient of acetonitrile in 0.2% taqueous trifluoroacetic acid). Product containing fractions were combined and concentrated in vacuo and the residue was further purified by preparative HPLC (eluting with a gradient of acetonitrile in 0.2% aqueous ammonia]. Pure product containing fractions were concentrated and the residue was dissolved in ethanol (0.5 mL), the solution acidified with ethereal HCl and the solvent removed to afford the title compound as a white solid (8 mg).

MS (APCI+) 551 (M+H)⁺

¹H NMR (399.825 MHz, CD₃OD) δ 7.54-7.46 (m, 2H), 7.28-7.24 (m, 1H), 6.75 (d, J=8.5 Hz, 1H), 6.48 (d, J=8.2 Hz, 1H), 4.61 (s, 2H), 3.87-3.78 (m, 1H), 3.66-3.58 (m, 1H), 3.49-3.41 (m, 1H), 3.35-3.27 (m, 4H), 3.21-3.14 (m, 4H), 3.08-3.02 (m, 2H), 2.98-2.82 (m, 4H), 1.64-1.52 (m, 2H), 1.23 (d, J=6.4 Hz, 3H), 0.91 (t, J=7.4 Hz, 3H).

### EXAMPLE 59

### (R)-3-(3,4-Dichlorophenethylamino)-N-(2-(2-(5-hydroxy-3-oxo-3,4-dihydro-2H-benzo[b][1,4]oxazin-8-yl)ethylamino)ethyl)-N-(3-methylbutan-2-yl)propanamide dihydrochloride

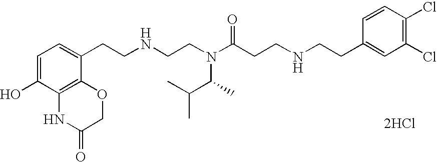

Prepared by an analogous procedure to Example 58, with appropriate substitution of reagents.

¹H NMR (399.825 MHz, CD₃OD) δ 7.54-7.46 (m, 2H), 7.28-7.24 (m, 1H), 6.75 (d, J=8.2 Hz, 1H), 6.48 (d, J=8.2 Hz, 1H), 4.61 (s, 2H), 3.75-3.66 (m, 1H), 3.53-3.44 (m, 1H), 3.41-3.28 (m, 5H), 3.21-3.15 (m, 4H), 3.08-3.03 (m, 2H), 2.98-2.79 (m, 4H), 1.82-1.72 (m, 1H), 1.27 (d, J=6.7 Hz, 3H), 0.99 (d, J=6.7 Hz, 3H), 0.87 (d, J=6.7 Hz, 3H).

### EXAMPLE 60-61

Prepared by analogous procedures to Example 24, with appropriate substitution of reagents.

### EXAMPLE 60

### (R)—N-(Heptan-2-yl)-N-(2-(2-(5-hydroxy-3-oxo-3,4-dihydro-2H-benzo[b][1,4]oxazin-8-yl)ethylamino)ethyl)-3-(phenethylamino)propanamide bis(trifluoroacetate)

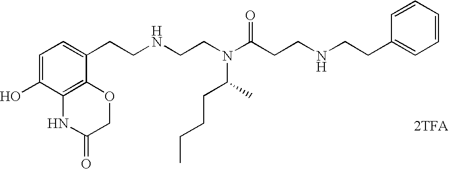

¹H NMR (399.825 MHz, CD₃OD) δ 7.35-7.22 (m, 5H), 6.72 (d, J=8.5 Hz, 1H), 6.48 (d, J=8.2 Hz, 1H), 4.60 (s, 2H), 3.90-3.80 (m, 1H), 3.63-3.55 (m, 1H), 3.49-3.40 (m, 1H), 3.34-3.25 (m, 4H), 3.21-3.12 (m, 4H), 3.04-2.98 (m, 2H), 2.93-2.77 (m, 4H), 1.56-1.49 (m, 2H), 1.37-1.14 (m, 9H), 0.92-0.86 (m, 3H).

### EXAMPLE 61

### (R)—N-(1-Cyclohexylethyl)-N-(2-(2-(5-hydroxy-3-oxo-3,4-dihydro-2H-benzo[b][1,4]oxazin-8-yl)ethylamino)ethyl)-3-(phenethylamino)propanamide bis(trifluoroacetate)

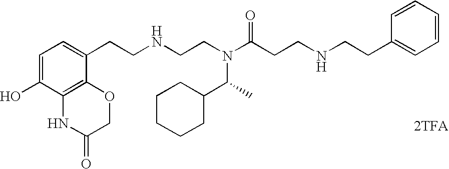

¹H NMR (399.825 MHz, CD₃OD) δ 7.35-7.22 (m, 5H), 6.72 (d, J=8.4 Hz, 1H), 6.48 (d, J=8.4 Hz, 1H), 4.60 (s, 2H), 3.70-3.61 (m, 1H), 3.56-3.47 (m, 1H), 3.39-3.25 (m, 5H), 3.22-3.13 (m, 4H), 3.04-2.98 (m, 2H), 2.91 (t, J=7.8 Hz, 2H), 2.84 (t, J=6.6 Hz, 2H), 1.85-0.81 (m, 14H).

### EXAMPLEs 62-68

Prepared by analogous procedures to Example 27, except Step ii was accomplished using a 1:2 mixture of 2 N aqueous hydrochloric acid and acetone (2.55 eq of HCl used) and Step iv was carried out using microwave heating in a CEM Discover microwave at 100° C. Reagents were substituted appropriately as required.

### EXAMPLE 62

### N-Cycloheptyl-3-(4-fluoro-3-methylphenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

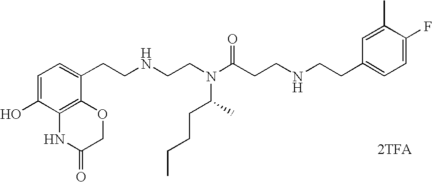

¹H NMR (399.825 MHz, CD₃OD) δ 7.17-7.13 (m, 1H), 7.12-7.07 (m, 1H), 6.97 (t, J=9.0 Hz, 1H), 6.71 (d, J=8.5 Hz, 1H), 6.48 (d, J=8.5 Hz, 1H), 4.60 (s, 2H), 3.77-3.69 (m, 1H), 3.58-3.53 (m, 2H), 3.32-3.25 (m, 4H), 3.20-3.12 (m, 4H), 2.98-2.85 (m, 6H), 2.23 (d, J=2.1 Hz, 3H), 1.85-1.45 (m, 12H).

### EXAMPLE 63

### 3-(4-Chlorophenethylamino)-N-cycloheptyl-N-(2-(2-(5-hydroxy-3-oxo-3,4-dihydro-2H-benzo[b][1,4]oxazin-8-yl)ethylamino)ethyl)propanamide bis(trifluoroacetate)

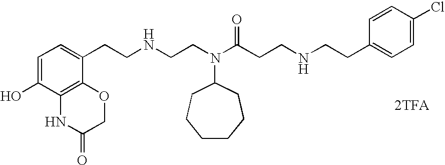

¹H NMR (399.825 MHz, CD₃OD) δ 7.35-7.24 (m, 4H), 6.71 (d, J=8.5 Hz, 1H), 6.47 (d, J=8.2 Hz, 1H), 4.60 (s, 2H), 3.77-3.68 (m, 1H), 3.58-3.51 (m, 2H), 3.35-3.27 (m, 4H), 3.20-3.12 (m, 4H), 3.03-2.97 (m, 2H), 2.93-2.85 (m, 4H), 1.86-1.44 (m, 12H).

### EXAMPLE 64

### N-Cycloheptyl-3-(2,5-difluorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

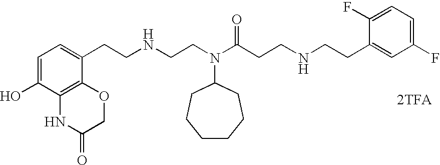

¹H NMR (399.825 MHz, CD₃OD) δ 7.16-7.09 (m, 2H), 7.07-6.99 (m, 1H), 6.71 (d, J=8.2 Hz, 1H), 6.47 (d, J=8.2 Hz, 1H), 4.60 (s, 2H), 3.78-3.68 (m, 1H), 3.58-3.53 (m, 2H), 3.35-3.27 (m, 4H), 3.20-3.14 (m, 4H), 3.09-3.04 (m, 2H), 2.93-2.86 (m, 4H), 1.86-1.44 (m, 12H).

### EXAMPLE 65

### N-Cycloheptyl-3-(3,5-difluorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

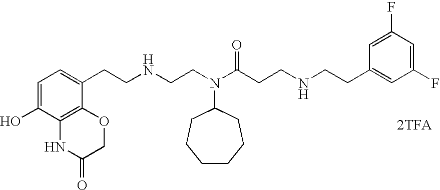

¹H NMR (399.825 MHz, CD₃OD) δ 6.97-6.91 (m, 2H), 6.88-6.82 (m, 1H), 6.71 (d, J=8.9 Hz, 1H), 6.48 (d, J=8.4 Hz, 1H), 4.60 (s, 2H), 3.78-3.69 (m, 1H), 3.58-3.53 (m, 2H), 3.37-3.26 (m, 4H), 3.20-3.13 (m, 4H), 3.06-3.01 (m, 2H), 2.93-2.86 (m, 4H), 1.85-1.45 (m, 12H).

### EXAMPLE 66

### 3-(3-Chloro-5-fluorophenethylamino)-N-cycloheptyl-N-(2-(2-(5-hydroxy-3-oxo-3,4-dihydro-2H-benzo[b][1,4]oxazin-8-yl)ethylamino)ethyl)propanamide bis(trifluoroacetate)

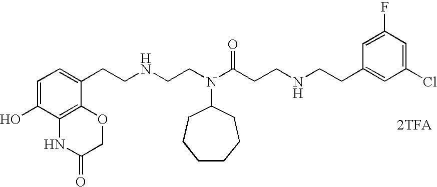

¹H NMR (399.825 MHz, CD₃OD) δ 7.20-7.19 (m, 1H), 7.13-7.09 (m, 1H), 7.07-7.03 (m, 1H), 6.71 (d, J=8.4 Hz, 1H), 6.48 (d, J=8.4 Hz, 1H), 4.60 (s, 2H), 3.78-3.69 (m, 1H), 3.58-3.53 (m, 2H), 3.36-3.25 (m, 4H), 3.20-3.13 (m, 4H), 3.05-2.99 (m, 2H), 2.93-2.86 (m, 4H), 1.86-1.43 (m, 12H).

### EXAMPLE 67

### N-Cycloheptyl-3-(3,4-difluorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

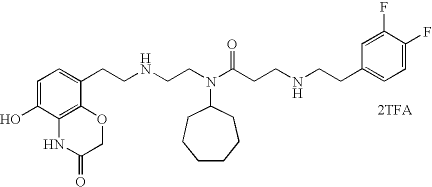

¹H NMR (399.825 MHz, CD₃OD) δ 7.27-7.18 (m, 2H), 7.11-7.07 (m, 1H), 6.71 (d, J=8.5 Hz, 1H), 6.47 (d, J=8.5 Hz, 1H), 4.60 (s, 2H), 3.77-3.68 (m, 1H), 3.58-3.53 (m, 2H), 3.34-3.27 (m, 4H), 3.20-3.13 (m, 4H), 3.03-2.97 (m, 2H), 2.93-2.85 (m, 4H), 1.85-1.45 (m, 12H).

### EXAMPLE 68

### 3-(3-Chlorophenethylamino)-N-cycloheptyl-N-(2-(2-(5-hydroxy-3-oxo-3,4-dihydro-2H-benzo[b][1,4]oxazin-8-yl)ethylamino)ethyl)propanamide bis(trifluoroacetate)

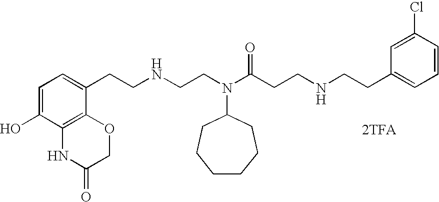

¹H NMR (399.825 MHz, CD₃OD) δ 7.35-7.19 (m, 4H), 6.71 (d, J=8.2 Hz, 1H), 6.48 (d, J=8.2 Hz, 1H), 4.60 (s, 2H), 3.78-3.69 (m, 1H), 3.58-3.53 (m, 2H), 3.33-3.26 (m, 4H), 3.20-3.13 (m, 4H), 3.04-2.98 (m, 2H), 2.93-2.85 (m, 4H), 1.85-1.44 (m, 12H).

### EXAMPLE 69

### N-Cycloheptyl-3-(3-fluorophenethylamino)-N-(2-(2-(5-hydroxy-3-oxo-3,4-dihydro-2H-benzo[b][1,4]oxazin-8-yl)ethylamino)ethyl)propanamide bis(trifluoroacetate)

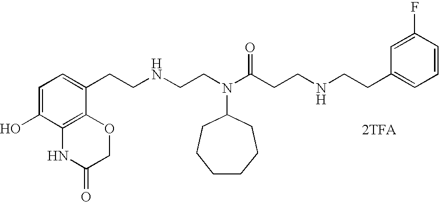

Prepared by an analogous procedure to Example 27, with appropriate substitution of reagents.

¹H NMR (399.826 MHz, DMSO) δ 9.96-9.80 (m, 2H), 8.57-8.47 (m, 4H), 7.42-7.33 (1,1H), 7.17-7.07 (m, 3H), 6.68-6.63 (m, 1H), 6.51-6.45 (m, 1H), 4.54 (s, 2H), 3.71-3.63 (m, 1H), 3.47-3.40 (m, 2H), 3.30-3.21 (m, 2H), 3.21-3.13 (m, 2H), 3.12-3.00 (m, 4H), 3.00-2.92 (m, 2H), 2.84-2.72 (m, 4H), 1.79-1.36 (m, 12H).

### EXAMPLE 70

### N-Cyclohexyl-N-(2-(2-(5-hydroxy-3-oxo-3,4-dihydro-2H-benzo[b][1,4]oxazin-8-yl)ethylamino)ethyl)-3-phenethoxypropanamide trifluoroacetate

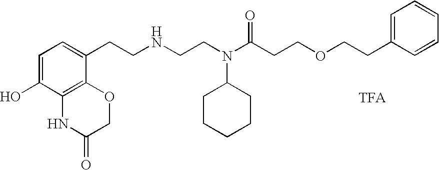

Prepared by an analogous procedure to Example 2, using 8-(2-aminoethyl)-5-hydroxy-2H-benzo[b][1,4]oxazin-3(4H)-one hydrochloride [Example 23, Step vii] in place of 5-(2-aminoethyl)-8-hydroxyquinolin-2(1H)-one hydrochloride.

MS (APCI+) 510 [M+H]⁺

¹H NMR (399.826 MHz, DMSO) δ 9.97-9.91 (m, 1H), 9.88-9.81 (m, 1H), 8.45-8.32 (m, 2H), 7.30-7.15 (m, 5H), 6.65 (d, J=8.2 Hz, 1H), 6.47 (d, J=8.2 Hz, 1H), 4.54 (s, 2H), 3.68-3.55 (m, 5H), 3.41 (t, J=8.1 Hz, 2H), 3.11-2.89 (m, 4H), 2.82-2.73 (m, 4H), 2.61 (t, J=6.6 Hz, 2H), 1.79-1.56 (m, 4H), 1.53-1.20 (m, 5H), 1.13-0.97 (m, 1H).

### EXAMPLE 71

### N-Cyclohexyl-3-(4-fluorophenethoxy)-N-(2-(2-(8-hydroxy-2-oxo-1,2-dihydroquinolin-5-yl)ethylamino)ethyl)propanamide hydrochloride

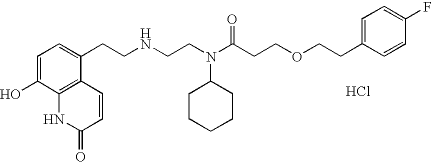

Prepared by an analogous procedure to Example 2, using 3-(4-fluorophenethoxy)propanoic acid [WO1997010227] in place of 3-phenethoxypropanoic acid in Step ii. Salt formation was achieved by treatment of the free base with methanol and 2 M hydrogen chloride in diethyl ether. After concentration in vacuo, the residue was crystallised from a mixture of ethyl acetate and methanol.

MS (APCI+) 524 [M+H]⁺

¹H NMR (399.825 MHz, CD₃OD) δ 8.29 (d, J=9.5 Hz, 1H), 7.18 (dd, J=8.5, 5.6 Hz, 2H), 7.08 (d, J=8.2 Hz, 1H), 7.01 (d, J=7.9 Hz, 1H), 6.94 (t, J=8.7 Hz, 2H), 6.76 (d, J=9.7 Hz, 1H), 3.76-3.68 (m, 1H), 3.70 (t, J=6.2 Hz, 2H), 3.64 (t, J=6.7 Hz, 2H), 3.55 (t, J=5.6 Hz, 2H), 3.26-3.20 (m, 4H), 3.10 (t, J=5.3 Hz, 2H), 2.80 (t, J=6.7 Hz, 2H), 2.65 (t, J=6.0 Hz, 2H), 1.81 (d, J=12.6 Hz, 2H), 1.74-1.61 (m, 3H), 1.52-1.29 (m, 4H), 1.21-1.07 (m, 1H).

### EXAMPLE 72

### N-Cyclohexyl-3-(3-fluorophenethoxy)-N-(2-(2-(8-hydroxy-2-oxo-1,2-dihydroquinolin-5-yl)ethylamino)ethyl)propanamide hydrochloride

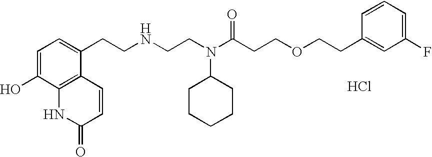

### Step i) N-Cyclohexyl-N-(2,2-dimethoxyethyl)-3-(3-fluorophenethoxy)propanamide

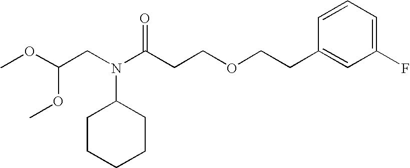

Prepared by an analogous procedure to Example 2, Step ii, using 3-(3-fluorophenethoxy)propanoic acid [WO2007018461] in place of 3-phenethoxypropanoic acid.

MS (APCI+) 382 [M+H]⁺

### Step ii) N-Cyclohexyl-3-(3-fluorophenethoxy)-N-(2-(2-(8-hydroxy-2-oxo-1,2-dihydroquinolin-5-yl)ethylamino)ethyl)propanamide hydrochloride

2 N Hydrochloric acid (5 mL) was added to a mixture of N-cyclohexyl-N-(2,2-dimethoxyethyl)-3-(3-fluorophenethoxy)propanamide (600 mg) [Step i] in acetone (10 mL). The reaction was stirred for 4 h then concentrated in vacuo to leave a residual aqueous phase which was extracted with dichloromethane (×3). The combined organics were washed with water, dried over sodium sulfate, filtered and concentrated in vacuo. The residue was added to a stirred solution of 5-(2-aminoethyl)-8-hydroxyquinolin-2(1H)-one hydrochloride (200 mg) [J. Med. Chem. 1985, 28, 1803] and sodium bicarbonate (69.8 mg) in NMP (10 mL) and water (0.5 mL), which had been pre-stirred for 5 minutes. After 10 minutes, sodium triacetoxyborohydride (352 mg) was added and stirring continued for 18 h. Saturated aqueous sodium bicarbonate was added and the mixture was extracted with ethyl acetate (×2). The combined organics were dried over sodium sulfate, filtered and concentrated in vacuo. The residue was loaded onto an SCX cartridge and the solid phase was washed with methanol prior to elution with 10% concentrated aqueous ammonia in methanol. The basic solution was concentrated in vacuo and the residue was purified by reverse phase preparative HPLC (eluting with a 5-50% gradient of acetonitrile in 0.2% aqueous ammonia). Product containing fractions were combined and concentrated in vacuo and the residue was treated with a 2 N solution of hydrogen chloride in diethyl ether. The mixture was concentrated in vacuo and the residue crystallised from a mixture of ethyl acetate and methanol. The resulting solid material was isolated by filtration and dried under vacuum to afford the title compound as white solid (45 mg).

MS (APCI+) 524 [M+H]⁺

¹H NMR (399.825 MHz, CD₃OD) δ 8.34 (d, J=9.7 Hz, 1H), 7.26-7.20 (m, 1H), 7.12-7.01 (m, 2H), 7.01-6.98 (m, 1H), 6.96-6.92 (m, 1H), 6.89-6.83 (m, 1H), 6.81 (d, J=9.7 Hz, 1H), 3.74-3.64 (m, 5H), 3.57-3.52 (m, 2H), 3.26-3.21 (m, 4H), 3.13-3.08 (m, 2H), 2.86-2.81 (m, 2H), 2.68-2.63 (m, 2H), 1.84-1.77 (m, 2H), 1.73-1.61 (m, 3H), 1.51-1.29 (m, 4H), 1.20-1.07 (m, 1H).

### EXAMPLE 73

### N-Cyclohexyl-3-(2-fluorophenethoxy)-N-(2-(2-(8-hydroxy-2-oxo-1,2-dihydroquinolin-5-yl)ethylamino)ethyl)propanamide hydrochloride

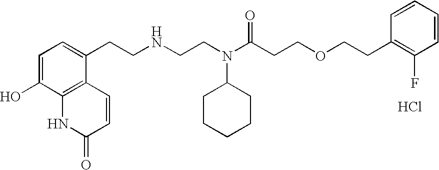

### Step i) 3-(2-Fluorophenethoxy)propanoic acid

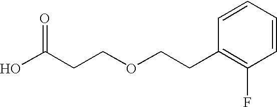

Prepared by an analogous procedure to that described for 3-(4-fluorophenethoxy)propanoic acid in WO1997070227, using 2-(2-fluorophenyl)ethanol in place of 2-(4-fluorophenyl)ethanol.

¹H NMR (299.946 MHz, CDCl₃) δ 7.28-7.14 (m, 2H), 7.10-6.96 (m, 2H), 3.75 (t, J=6.1 Hz, 2H), 3.71 (t, J=6.9 Hz, 2H), 2.94 (t, J=6.9 Hz, 2H), 2.64 (t, J=5.8 Hz, 2H).

### Step ii) N-Cyclohexyl-3-(2-fluorophenethoxy)-N-(2-(2-(8-hydroxy-2-oxo-1,2-dihydroquinolin-5-yl)ethylamino)ethyl)propanamide hydrochloride

Prepared by an analogous procedure to Example 2, using 3-(2-fluorophenethoxy)propanoic acid [Step i] in place of 3-phenethoxypropanoic acid in Step ii. The initially formed product was converted to the hydrochloride salt as described in Example 71.

MS (APCI+) 524 [M+H]⁺

¹H NMR (399.825 MHz, CD₃OD) δ 8.43 (d, J=9.2 Hz, 1H), 7.27-7.12 (m, 3H), 7.05 (d, J=7.9 Hz, 2H), 7.03-6.95 (m, 1H), 6.86 (d, J=9.5 Hz, 1H), 3.80-3.60 (m, 1H), 3.72 (t, J=6.0 Hz, 2H), 3.65 (t, J=6.8 Hz, 2H), 3.55 (t, J=5.4 Hz, 2H), 3.31-3.19 (m, 4H), 3.11 (t, J=5.3 Hz, 2H), 2.86 (t, J=6.7 Hz, 2H), 2.65 (t, J=6.0 Hz, 2H), 1.86-1.06 (m, 10H).

### EXAMPLE 74

### N-Cyclohexyl-3-(3-fluorophenethoxy)-N-(2-(2-(5-hydroxy-3-oxo-3,4-dihydro-2H-benzo[b][1,4]oxazin-8-yl)ethylamino)ethyl)propanamide trifluoroacetate

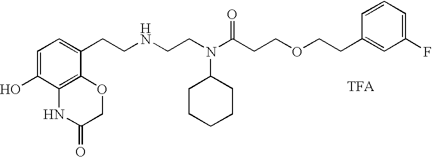

Prepared by an analogous procedure to Example 72, Step ii, using 8-(2-aminoethyl)-5-hydroxy-2H-benzo[b][1,4]oxazin-3(4H)-one hydrochloride [Example 23, Step vii] in place of 5-(2-aminoethyl)-8-hydroxyquinolin-2(1H)-one hydrochloride.

MS (APCI+) 528 [M+H]⁺

¹H NMR (299.947 MHz, CD₃OD) δ 7.35-7.23 (m, 1H), 7.13-6.87 (m, 3H), 6.82-6.72 (m, 1H), 6.58-6.49 (m, 1H), 4.71-4.62 (m, 2H), 3.83-3.67 (m, 5H), 3.63-3.52 (m, 2H), 3.27-3.05 (m, 4H), 3.02-2.85 (m, 4H), 2.75-2.64 (m, 2H), 1.93-1.80 (m, 2H), 1.79-1.64 (m, 3H), 1.57-1.30 (m, 4H), 1.28-1.08 (m, 1H).

### EXAMPLE 75

### N-Cyclohexyl-3-(4-fluorophenethoxy)-N-(2-(2-(5-hydroxy-3-oxo-3,4-dihydro-2H-benzo[b][1,4]oxazin-8-yl)ethylamino)ethyl)propanamide trifluoroacetate

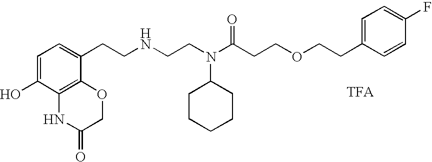

### Step i) N-Cyclohexyl-N-(2,2-dimethoxyethyl)-3-(4-fluorophenethoxy)propanamide

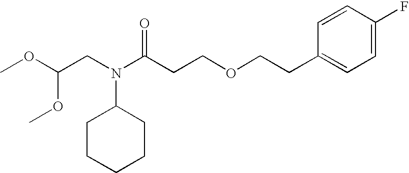

Prepared by an analogous procedure to Example 2, Step ii, using 3-(4-fluorophenethoxy)propanoic acid [WO1997010227] in place of 3-phenethoxypropanoic acid.

MS (APCI+) 382 [M+H]⁺

### Step ii) N-Cyclohexyl-3-(4-fluorophenethoxy)-N-(2-(2-(5-hydroxy-3-oxo-3,4-dihydro-2H-benzo[b][1,4]oxazin-8-yl)ethylamino)ethyl)propanamide trifluoroacetate

p-Toluenesulfonic acid (0.299 g) was added to a solution of N-cyclohexyl-N-(2,2-dimethoxyethyl)-3-(4-fluorophenethoxy)propanamide (0.2 g) [Step i] in dichloromethane (5 mL) and the solution was stirred for 1 h. Further dichloromethane (15 mL) was added and the mixture washed with saturated aqueous sodium bicarbonate and water, dried over sodium sulfate, filtered and concentrated in vacuo. The residue was added to a pre-mixed solution of sodium bicarbonate (0.034 g) and 8-(2-aminoethyl)-5-hydroxy-2H-benzo[b][1,4]oxazin-3(4H)-one, HCl (0.1 g) [Example 23, Step vii] in NMP (5 mL) and water (0.5 mL), which had been stirred for 15 min. The resulting mixture was stirred for 15 minutes, then sodium triacetoxyborohydride (0.130 g) was added and the reaction stirred for a further 20 h. The mixture was purified by reverse phase preparative HPLC (eluting with a gradient of acetonitrile in 0.2% aqueous trifluoroacetic acid) to afford the title compound as a white solid (0.070 g).

MS (APCI+) 528 [M+H]⁺

¹H NMR (299.947 MHz, CD₃OD) δ 7.23 (m, 2H), 6.99 (m, 2H), 6.74 (d, J=8.8 Hz, 1H), 6.51 (d, J=8.8 Hz, 1H), 4.65 (s, 2H), 3.79-3.62 (m, 5H), 3.55 (t, J=5.5 Hz, 2H), 3.18 (t, J=7.2 Hz, 2H), 3.09 (t, J=5.7 Hz, 2H), 2.92 (t, J=7.4 Hz, 2H), 2.84 (t, J=6.9 Hz, 2H), 2.66 (t, J=6.2 Hz, 2H), 1.84 (m, 2H), 1.70 (m, 3H), 1.55-1.29 (m, 4H), 1.17 (m, 1H).

### EXAMPLE 76

### N-Cyclohexyl-3-(2-fluorophenethoxy)-N-(2-(2-(5-hydroxy-3-oxo-3,4-dihydro-2H-benzo[b][1,4]oxazin-8-yl)ethylamino)ethyl)propanamide trifluoroacetate

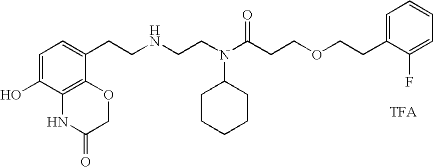

Prepared by an analogous procedure to Example 75 using 3-(2-fluorophenethoxy)propanoic acid [Example 73, Step i] in place of 3-phenethoxypropanoic acid.

MS (APCI+) 528 [M+H]⁺

¹H NMR (399.825 MHz, CD₃OD) δ 7.27-6.97 (m, 4H), 6.71 (d, J=8.5 Hz, 1H), 6.47 (d, J=8.5 Hz, 1H), 4.61 (s, 2H), 3.72 (m, 1H), 3.70 (t, J=6.0 Hz, 2H), 3.65 (t, J=6.6 Hz, 2H), 3.51 (t, J=5.8 Hz, 2H), 3.14 (t, J=7.5 Hz, 2H), 3.05 (t, J=5.8 Hz, 2H), 2.88 (m, 4H), 2.63 (t, J=6.0 Hz, 2H), 1.84-1.76 (m, 2H), 1.73-1.61 (m, 3H), 1.49-1.28 (m, 4H),

### EXAMPLE 77

### 3-(3-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide trifluoroacetate

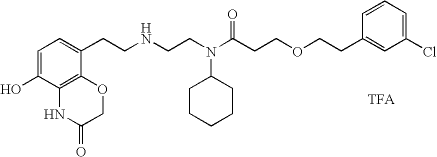

Sodium hydride (60% dispersion in mineral oil; 0.025 g) was added to a solution of 2-(3-chlorophenyl)ethanol (0.128 g) in DMF (1 mL) and the mixture was stirred for 15 minutes. tert-Butyl 2-(N-cyclohexylacrylamido)ethyl(2-(5-hydroxy-3-oxo-3,4-dihydro-2H-benzo[b][1,4]oxazin-8-yl)ethyl)carbamate (0.1 g) [Example 27, Step iii] was added and the mixture was stirred for a further 20 h, then diluted with EtOAc (20 mL) and washed with 2 M hydrochloric acid (20 mL), water and brine. The solution was dried over sodium sulfate, filtered and concentrated in vacuo. The residue was dissolved in dichloromethane (1 mL), trifluoroacetic acid (1.0 mL) was added and the solution was stirred for 2 h, then concentrated in vacuo. The residue was purified by reverse phase HPLC (eluting with a gradient of acetonitrile in 0.2% aqueous trifluoroacetic acid) to afford the title compound as a white solid (0.013 g).

MS (APCI+) 544 [M+H]⁺

¹H NMR (299.947 MHz, CD₃OD) δ 7.20 (m, 4H), 6.74 (d, J=8.5 Hz, 1H), 6.51 (d, J=8.5 Hz, 1H), 4.64 (s, 2H), 3.72 (m, 5H), 3.54 (t, J=5.8 Hz, 2H), 3.17 (t, J=7.2 Hz, 2H), 3.08 (t, J=5.8 Hz, 2H), 2.91 (t, J=7.1 Hz, 2H), 2.85 (t, J=6.2 Hz, 2H), 2.65 (t, J=6.2 Hz, 2H), 1.83 (m, 2H), 1.69 (m, 3H), 1.54-1.29 (m, 4H), 1.17 (m, 1H).

### EXAMPLE 78

### N-Cyclohexyl-3-(3,5-difluorophenethoxy)-N-(2-(2-(8-hydroxy-2-oxo-1,2-dihydroquinolin-5-yl)ethylamino)ethyl)propanamide trifluoroacetate

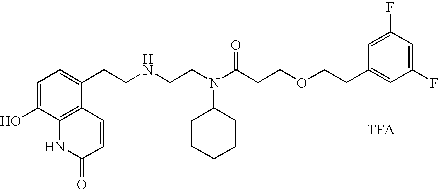

### Step i) 3-(3,5-Difluorophenethoxy)propanoic acid

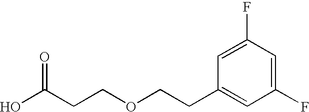

Prepared by an analogous procedure to that described for 3-(4-fluorophenethoxy)propanoic acid in WO1997010227, using 2-(3,5-difluorophenyl)ethanol in place of 2-(4-fluorophenyl)ethanol.

¹H NMR (399.825 MHz, CD₃OD) δ 6.86-6.78 (m, 2H), 6.75-6.68 (m, 1H), 3.70-3.62 (m, 4H), 2.86-2.80 (m, 2H), 2.54-2.46 (m, 2H).

### Step ii) N-Cyclohexyl-3-(3,5-difluorophenethoxy)-N-(2,2-dimethoxyethyl)propanamide

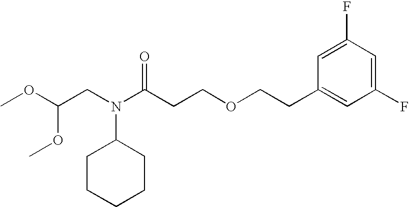

Prepared by an analogous procedure to Example 2, Step ii, using 3-(3,5-difluorophenethoxy)propanoic acid [Step i] in place of 3-phenethoxypropanoic acid.

MS (APCI+) 400 [M+H]⁺

### Step iii) N-Cyclohexyl-3-(3,5-difluorophenethoxy)-N-(2-(2-(8-hydroxy-2-oxo-1,2-dihydroquinolin-5-yl)ethylamino)ethyl)propanamide trifluoroacetate

Prepared by an analogous procedure to Example 72, Step ii) using N-cyclohexyl-3-(3,5-difluorophenethoxy)-N-(2,2-dimethoxyethyl)propanamide in place of N-Cyclohexyl-N-(2,2-dimethoxyethyl)-3-(3-fluorophenethoxy)propanamide. The hydrochloride salt was further purified by reverse phase preparative HPLC (eluting with a gradient of acetonitrile in 0.2% aqueous trifluoroacetic acid). Pure product-containing fractions were concentrated in vacuo to afford the title compound as a white solid.

MS (APCI+) 542 [M+H]⁺

¹H NMR (399.825 MHz, CD₃OD) δ 8.15 (d, J=9.7 Hz, 1H), 6.98 (dd, J=19.9, 8.7 Hz, 2H), 6.84-6.77 (m, 2H), 6.74-6.65 (m, 2H), 3.75-3.64 (m, 5H), 3.56-3.51 (m, 2H), 3.21 (s, 4H), 3.12-3.07 (m, 2H), 2.84 (t, J=6.1 Hz, 2H), 2.65 (t, J=6.1 Hz, 2H), 1.84-1.76 (m, 2H), 1.72-1.61 (m, 3H), 1.49-1.26 (m, 4H), 1.19-1.06 (m, 1H).

### EXAMPLE 79

### (R)—N-sec-Butyl-N-(2-(2-(8-hydroxy-2-oxo-1,2-dihydroquinolin-5-yl)ethylamino)ethyl)-3-phenethoxypropanamide hydrochloride

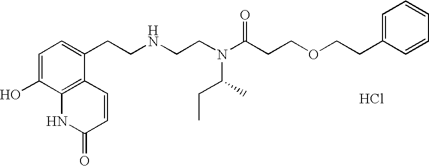

### Step i) (R)—N-(2,2-Dimethoxyethyl)butan-2-amine

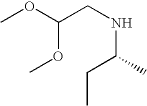

Prepared by an analogous procedure to Example 2, Step i, using (R¹⁵)-butan-2-amine in place of cyclohexylamine.

¹H NMR (399.824 MHz, CDCl₃) δ 4.47 (td, J=5.6, 0.8 Hz, 1H), 3.39 (d, J=1.0 Hz, 6H), 2.79-2.66 (m, 2H), 2.59-2.50 (m, 1H), 1.55-1.44 (m, 1H), 1.38-1.26 (m, 1H), 1.04 (dd, J=6.3, 0.9 Hz, 3H), 0.92-0.87 (m, 3H).

### Step ii) 3-Phenethoxypropanoyl chloride

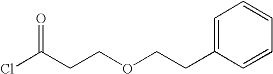

Oxalyl chloride (15.25 mL) and DMF (0.1 mL) were added sequentially to a solution of 3-phenethoxypropanoic acid (16.92 g) [Tetrahedron 1998, 54, 12151-60] in dichloromethane (100 mL) and the mixture was stirred at ambient temperature for 2 h. The solvent was removed in vacuo to afford 3-phenethoxypropanoyl chloride as a yellow oil (15.02 g). The material was used immediately.

### Step iii) (R)—N-sec-Butyl-N-(2,2-dimethoxyethyl)-3-phenethoxypropanamide

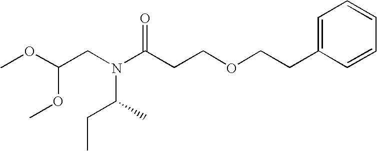

Triethylamine (0.864 mL) was added to a stirred solution of (R)—N-(2,2-dimethoxyethyl)butan-2-amine (1 g) [Step i] in dichloromethane (10 mL) and the mixture was cooled to 0° C. A solution of 3-phenethoxypropanoyl chloride (1.319 g) [Step ii] in dichloromethane (9.61 mL) was added over 5 minutes under nitrogen. When the addition was complete, the mixture was stirred at ambient temperature for 18 h, then diluted with water and extracted into dichloromethane (2×50 mL). The combined extracts were washed with water, dried over sodium sulfate, filtered and concentrated in vacuo. The residue was purified by flash chromatography on silica (eluting with 15 to 30, to 50% ethyl acetate in iso-hexane) to afford the sub-titled compound as a colourless oil (1.020 g).

¹H NMR (399.824 MHz, CDCl₃) δ 7.31-7.16 (m, 5H), 4.68-4.37 (m, 1H), 3.81-3.63 (m, 5H), 3.43-3.13 (m, 8H), 2.90-2.84 (m, 2H), 2.73-2.62 (m, 2H), 1.65-1.39 (m, 2H), 1.18-1.14 (m, 3H), 0.88-0.81 (m, 3H).

### Step iv) (R)—N-sec-Butyl-N-(2-(2-(8-hydroxy-2-oxo-1,2-dihydroquinolin-5-yl)ethylamino)ethyl)-3-phenethoxypropanamide hydrochloride

Prepared by an analogous procedure to Example 72, Step ii, using (R)—N-sec-butyl-N-(2,2-dimethoxyethyl)-3-phenethoxypropanamide [Step iii] in place of N-cyclohexyl-N-(2,2-dimethoxyethyl)-3-(3-fluorophenethoxy)propanamide.

MS (APCI+) 480 [M+H]⁺

¹H NMR (399.825 MHz, CD₃OD) δ 8.51 (d, J=9.7 Hz, 1H), 7.24-7.07 (m, 7H), 6.93 (d, J=9.7 Hz, 1H), 3.97-3.86 (m, 1H), 3.73-3.53 (m, 5H), 3.47-3.38 (m, 1H), 3.33-3.07 (m, 6H), 2.81 (t, J=6.9 Hz, 2H), 2.73-2.56 (m, 2H), 1.57-1.47 (m, 2H), 1.16 (d, J=6.7 Hz, 3H), 0.86 (t, J=7.4 Hz, 3H).

### EXAMPLEs 80-84

Prepared by analogous procedures to Example 79, with appropriate substitution of reagents. Compounds prepared as the trifluoroacetate salt were purified a second time by reverse phase HPLC (eluting with a gradient of acetonitrile in 0.2% aqueous trifluoroacetic to acid). Pure product-containing fractions were combined and concentrated in vacuo to afford the title compounds.

### EXAMPLE 80

### (R)—N-(2-(2-(8-Hydroxy-2-oxo-1,2-dihydroquinolin-5-yl)ethylamino)ethyl)-N-(3-methylbutan-2-yl)-3-phenethoxypropanamide hydrochloride

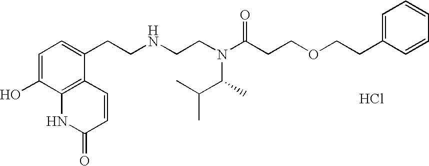

¹H NMR (399.825 MHz, CD₃OD) δ 8.36 (d, J=9.7 Hz, 1H), 7.24-7.09 (m, 6H), 7.03 (d, J=7.9 Hz, 1H), 6.82 (d, J=9.7 Hz, 1H), 3.77-3.55 (m, 6H), 3.40-3.32 (m, 1H), 3.31-3.05 (m, 6H), 2.84-2.78 (m, 2H), 2.76-2.67 (m, 1H), 2.60-2.52 (m, 1H), 1.78-1.67 (m, 1H), 1.19 (d, J=6.7 Hz, 3H), 0.97 (d, J=6.7 Hz, 3H), 0.83 (d, J=6.7 Hz, 3H).

### EXAMPLE 81

### N-Cycloheptyl-N-(2-(2-(8-hydroxy-2-oxo-1,2-dihydroquinolin-5-yl)ethylamino)ethyl)-3-phenethoxypropanamide trifluoroacetate

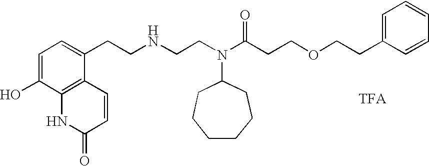

¹H NMR (399.825 MHz, CD₃OD) δ 8.15 (d, J=9.7 Hz, 1H), 7.24-7.10 (m, 5H), 7.03-6.95 (m, 2H), 6.67 (d, J=10.0 Hz, 1H), 3.91-3.81 (m, 1H), 3.71 (t, J=5.9 Hz, 2H), 3.66 (t, J=6.8 Hz, 2H), 3.50 (t, J=5.8 Hz, 2H), 3.21-3.18 (m, 4H), 3.12 (t, J=5.6 Hz, 2H), 2.82 (t, J=6.8 Hz, 2H), 2.65 (t, J=6.0 Hz, 2H), 1.80-1.43 (m, 12H).

### EXAMPLE 82

### N-(2-(2-(8-Hydroxy-2-oxo-1,2-dihydroquinolin-5-yl)ethylamino)ethyl)-N-isopropyl-3-phenethoxypropanamide trifluoroacetate

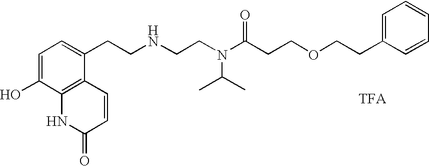

¹H NMR (399.825 MHz, CD₃OD) δ 8.16 (d, J=10.0 Hz, 1H), 7.25-7.10 (m, 5H), 7.04-6.95 (m, 2H), 6.68 (d, J=9.7 Hz, 1H), 4.25-4.18 (m, 1H), 3.72 (t, J=6.0 Hz, 2H), 3.67 (t, J=6.8 Hz, 2H), 3.51 (s, 2H), 3.22-3.19 (m, 4H), 3.12 (t, J=5.8 Hz, 2H), 2.83 (t, J=6.7 Hz, 2H), 2.66 (t, J=6.7 Hz, 2H), 1.17 (d, J=6.7 Hz, 6H).

### EXAMPLE 83

### (R)—N-(Hexan-2-yl)-N-(2-(2-(8-hydroxy-2-oxo-1,2-dihydroquinolin-5-yl)ethylamino)ethyl)-3-phenethoxypropanamide trifluoroacetate

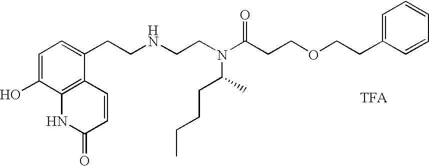

¹H NMR (399.825 MHz, CD₃OD) δ 8.15 (d, J=9.7 Hz, 1H), 7.25-7.10 (m, 5H), 7.04-6.96 (m, 2H), 6.68 (d, J=9.7 Hz, 1H), 4.04-3.94 (m, 1H), 3.72 (t, J=6.1 Hz, 2H), 3.67 (t, J=6.1 Hz, 2H), 3.59-3.51 (m, 1H), 3.46-3.38 (m, 1H), 3.23-3.20 (m, 4H), 3.14-3.09 (m, 2H), 2.83 (t, J=6.9 Hz, 2H), 2.73-2.56 (m, 2H), 1.52-1.46 (m, 2H), 1.37-1.14 (m, 7H), 0.91 (t, J=7.2 Hz, 3H).

### EXAMPLE 84

### N-(2-(2-(8-Hydroxy-2-oxo-1,2-dihydroquinolin-5-yl)ethylamino)ethyl)-N-neopentyl-3-phenethoxypropanamide trifluoroacetate

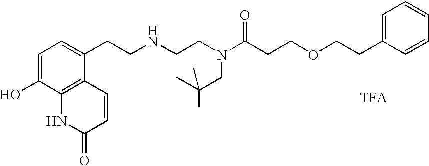

¹H NMR (399.825 MHz, CD₃OD) δ 8.14 (d, J=9.7 Hz, 1H), 7.24-7.07 (m, 5H), 7.03-6.95 (m, 2H), 6.69 (d, J=10.0 Hz, 1H), 3.78-3.60 (m, 6H), 3.23-3.10 (m, 8H), 2.80 (t, J=6.9 Hz, 2H), 2.62 (t, J=6.0 Hz, 2H), 0.99-0.92 (m, 9H).

### EXAMPLEs 85-87

Prepared by analogous procedures to Example 2. Suitably substituted phenethoxypropanoic acids were prepared as described for 3-(4-fluorophenethoxy)propanoic acid in WO1997010227. Salt formation was achieved by treatment of the free base with methanol and 2 M hydrogen chloride in diethyl ether. After concentration in vacuo, the residue was crystallised from a mixture of ethyl acetate and methanol.

### EXAMPLE 85

### N-Cyclohexyl-3-(2,4-difluorophenethoxy)-N-(2-(2-(8-hydroxy-2-oxo-1,2-dihydroquinolin-5-yl)ethylamino)ethyl)propanamide hydrochloride

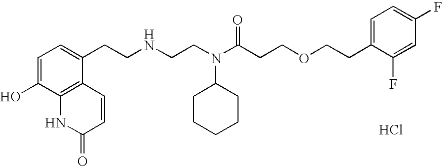

¹H NMR (399.825 MHz, CD₃OD) δ 8.34 (d, J=9.6 Hz, 1H), 7.26 (q, J=7.8 Hz, 1H), 7.10 (d, J=8.2 Hz, 1H), 7.02 (d, J=7.9 Hz, 1H), 6.88-6.77 (m, 3H), 3.76-3.66 (m, 1H), 3.71 (t, J=6.2 Hz, 2H), 3.63 (t, J=6.6 Hz, 2H), 3.55 (t, J=5.8 Hz, 2H), 3.27-3.20 (m, 4H), 3.11 (t, J=5.7 Hz, 2H), 2.83 (t, J=6.7 Hz, 2H), 2.65 (t, J=6.0 Hz, 2H), 1.85-1.76 (m, 2H), 1.75-1.61 (m, 3H), 1.53-1.07 (m, 5H).

### EXAMPLE 86

### N-Cyclohexyl-3-(2,3-difluorophenethoxy)-N-(2-(2-(8-hydroxy-2-oxo-1,2-dihydroquinolin-5-yl)ethylamino)ethyl)propanamide hydrochloride

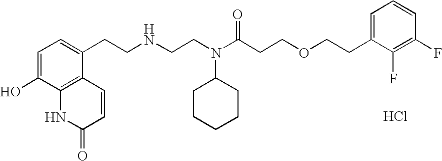

¹H NMR (399.825 MHz, CD₃OD) δ 8.42 (d, J=9.7 Hz, 1H), 7.15 (d, J=8.2 Hz, 1H), 7.09-7.01 (m, 4H), 6.86 (d, J=9.7 Hz, 1H), 3.76-3.66 (m, 1H), 3.72 (t, J=6.1 Hz, 2H), 3.67 (t, J=6.7 Hz, 2H), 3.55 (t, J=5.7 Hz, 2H), 3.30-3.20 (m, 4H), 3.11 (t, J=5.9 Hz, 2H), 2.90 (td, J=6.6, 1.2 Hz, 2H), 2.65 (t, J=6.2 Hz, 2H), 1.81 (d, J=13.1 Hz, 2H), 1.75-1.60 (m, 3H), 1.53-1.27 (m, 4H), 1.22-1.06 (m, 1H).

### EXAMPLE 87

### N-Cyclohexyl-3-(3,4-difluorophenethoxy)-N-(2-(2-(8-hydroxy-2-oxo-1,2-dihydroquinolin-5-yl)ethylamino)ethyl)propanamide hydrochloride

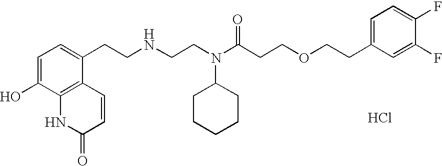

¹H NMR (399.825 MHz, CD₃OD) δ 8.42 (d, J=9.7 Hz, 1H), 7.17-7.02 (m, 4H), 7.00-6.94 (m, 1H), 6.85 (d, J=9.5 Hz, 1H), 3.77-3.66 (m, 1H), 3.70 (t, J=6.1 Hz, 2H), 3.65 (t, J=6.4 Hz, 2H), 3.56 (t, J=5.7 Hz, 2H), 3.31-3.20 ((m, 4H), 3.11 (t, J=5.7 Hz, 2H), 2.80 (t, J=6.3 Hz, 2H), 2.65 (t, J=6.1 Hz, 2H), 1.85-1.77 (m, 2H), 1.74-1.60 (m, 3H), 1.55-1.07 (m, 5H).

### EXAMPLEs 88-89

Prepared by analogous procedures to Example 72. Suitably substituted phenethoxypropanoic acids were prepared as described for 3-(4-fluorophenethoxy)propanoic acid in WO1997010227.

### EXAMPLE 88

### N-Cyclohexyl-3-(2,6-difluorophenethoxy)-N-(2-(2-(8-hydroxy-2-oxo-1,2-dihydroquinolin-5-yl)ethylamino)ethyl)propanamide hydrochloride

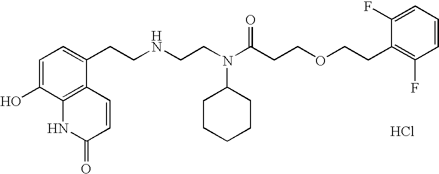

¹H NMR (399.825 MHz, CD₃OD) δ 8.51 (d, J=9.7 Hz, 1H), 7.25-7.17 (m, 2H), 7.09 (d, J=7.9 Hz, 1H), 6.95-6.85 (m, 3H), 3.75-3.66 (m, 3H), 3.62 (t, J=6.9 Hz, 2H), 3.56 (t, J=5.8 Hz, 2H), 3.35-3.27 (m, 2H), 3.26-3.20 (m, 2H), 3.12 (t, J=5.6 Hz, 2H), 2.91 (t, J=6.9 Hz, 2H), 2.65 (t, J=6.0 Hz, 2H), 1.85-1.77 (m, 2H), 1.75-1.61 (m, 3H), 1.53-1.27 (m, 4H), 1.21-1.08 (m, 1H).

### EXAMPLE 89

### N-Cyclohexyl-3-(2,5-difluorophenethoxy)-N-(2-(2-(8-hydroxy-2-oxo-1,2-dihydroquinolin-5-yl)ethylamino)ethyl)propanamide hydrochloride

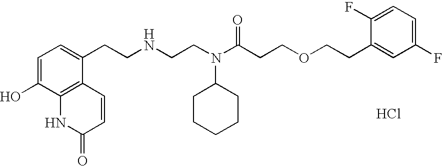

¹H NMR (399.825 MHz, CD₃OD) δ 8.43 (d, J=9.7 Hz, 1H), 7.15 (d, J=7.9 Hz, 1H), 7.08-6.98 (m, 3H), 6.93-6.85 (m, 2H), 3.76-3.64 (m, 5H), 3.58-3.53 (m, 2H), 3.30-3.20 (m, 4H), 3.14-3.08 (m, 2H), 2.89-2.82 (m, 2H), 2.68-2.63 (m, 2H), 1.84-1.77 (m, 2H), 1.74-1.59 (m, 3H), 1.52-1.25 (m, 4H), 1.20-1.09 (m, 1H).

### EXAMPLEs 90-92

Prepared by analogous procedures to Example 75. Suitably substituted phenethoxypropanoic acids were prepared as described for 3-(4-fluorophenethoxy)propanoic acid in WO1997010227.

### EXAMPLE 90

### N-cyclohexyl-3-(2,4-difluorophenethoxy)-N-(2-(2-(5-hydroxy-3-oxo-3,4-dihydro-2H-benzo[b][1,4]oxazin-8-yl)ethylamino)ethyl)propanamide trifluoroacetate

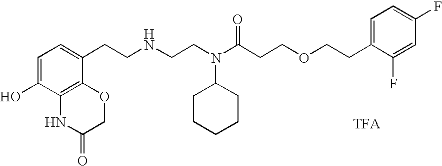

¹H NMR (299.947 MHz, CD₃OD) δ 7.30 (m, 1H), 6.88 (m, 2H), 6.74 (d, J=8.3 Hz, 1H), 6.51 (d, J=8.3 Hz, 1H), 4.64 (s, 2H), 3.75 (m, 1H), 3.73 (t, J=6.1 Hz, 2H), 3.66 (t, J=6.6 Hz, 2H), 3.54 (t, J=5.6 Hz, 2H), 3.18 (t, J=7.0 Hz, 2H), 3.09 (t, J=5.6 Hz, 2H), 2.92 (t, J=7.7 Hz, 2H), 2.87 (t, J=7.0 Hz, 2H), 2.66 (t, J=6.1 Hz, 2H), 1.89-1.79 (m, 2H), 1.76-1.63 (m, 3H), 1.55-1.29 (m, 4H), 1.16 (m, 1H).

### EXAMPLE 91

### N-Cyclohexyl-3-(3,4-difluorophenethoxy)-N-(2-(2-(5-hydroxy-3-oxo-3,4-dihydro-2H-benzo[b][1,4]oxazin-8-yl)ethylamino)ethyl)propanamide trifluoroacetate

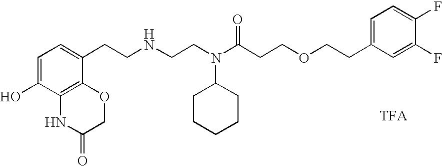

¹H NMR (299.947 MHz, CD₃OD) δ 7.26-7.09 (m, 2H), 7.01 (m, 1H), 6.74 (d, J=8.4 Hz, 1H), 6.50 (d, J=8.4 Hz, 1H), 4.64 (s, 2H), 3.78-3.64 (m, 5H), 3.58-3.46 (m, 2H), 3.18 (t, J=7.2 Hz, 2H), 3.09 (t, J=5.7 Hz, 2H), 2.92 (t, J=6.8 Hz, 2H), 2.83 (t, J=6.5 Hz, 2H), 2.66 (t, J=6.1 Hz, 2H), 1.88-1.79 (m, 2H), 1.76-1.64 (m, 3H), 1.55-1.30 (m, 4H), 1.19 (m, 1H).

### EXAMPLE 92

N-Cyclohexyl-3-(2,3-difluorophenethoxy)-N-(2-(2-(5-hydroxy-3-oxo-3,4-dihydro-2H-benzo[b][1,4]oxazin-8-yl)ethylamino)ethyl)propanamide trifluoroacetate

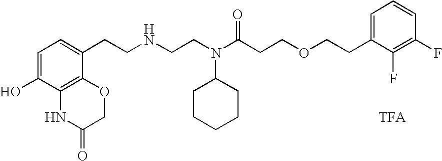

¹H NMR (299.947 MHz, CD₃OD) δ 7.08 (m, 3H), 6.74 (d, J=8.5 Hz, 1H), 6.50 (d, J=8.5 Hz, 1H), 4.64 (s, 2H), 3.72 (m, 5H), 3.54 (t, J=5.6 Hz, 2H), 3.18 (t, J=7.3 Hz, 2H), 3.09 (t, J=5.6 Hz, 2H), 2.93 (m, 4H), 2.65 (t, J=6.1 Hz, 2H), 1.88-1.79 (m, 2H), 1.76-1.62 (m, 3H), 1.54-1.29 (m, 4H), 1.15 (m, 1H).

### EXAMPLEs 93-96

Prepared by analogous procedures to Example 77, with appropriate substitution of reagents.

### EXAMPLE 93

### 3-(2-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide trifluoroacetate

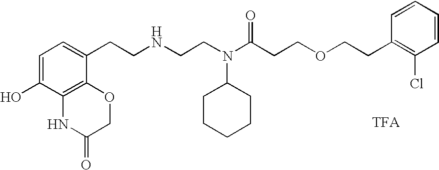

¹H NMR (399.825 MHz, CD₃OD) δ 7.30 (m, 2H), 7.18 (m, 2H), 6.71 (d, J=8.3 Hz, 1H), 6.47 (d, J=8.3 Hz, 1H), 4.61 (s, 2H), 3.74 (m, 1H), 3.72 (t, J=6.0 Hz, 2H), 3.67 (t, J=6.9 Hz, 2H), 3.51 (t, J=5.6 Hz, 2H), 3.15 (t, J=7.2 Hz, 2H), 3.06 (t, J=5.6 Hz, 2H), 2.97 (t, J=6.9 Hz, 2H), 2.89 (t, J=7.2 Hz, 2H), 2.64 (t, J=6.0 Hz, 2H), 1.84-1.76 (m, 2H), 1.72-1.61 (m, 3H), 1.49-1.26 (m, 4H), 1.19-1.07 (m, 1H).

### EXAMPLE 94

### 3-(4-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide trifluoroacetate

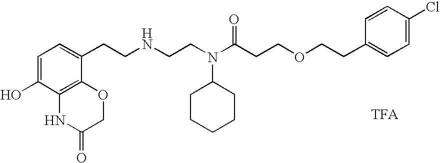

¹H NMR (399.825 MHz, CD₃OD) δ 7.25-7.16 (m, 4H), 6.71 (d, J=8.5 Hz, 1H), 6.47 (d, J=8.5 Hz, 1H), 4.61 (s, 2H), 3.72 (m, 1H), 3.68 (t, J=6.0 Hz, 2H), 3.64 (t, J=6.5 Hz, 2H), 3.50 (t, J=5.6 Hz, 2H), 3.14 (t, J=7.0 Hz, 2H), 3.04 (t, J=5.6 Hz, 2H), 2.88 (t, J=7.4 Hz, 2H), 2.80 (t, J=6.5 Hz, 2H), 2.62 (t, J=6.0 Hz, 2H), 1.84-1.77 (m, 2H), 1.71-1.61 (m, 3H), 1.48-1.28 (m, 4H), 1.19-1.06 (m, 1H).

### EXAMPLE 95

### N-Cycloheptyl-N-(2-(2-(5-hydroxy-3-oxo-3,4-dihydro-2H-benzo[b][1,4]oxazin-8-yl)ethylamino)ethyl)-3-phenethoxypropanamide trifluoroacetate

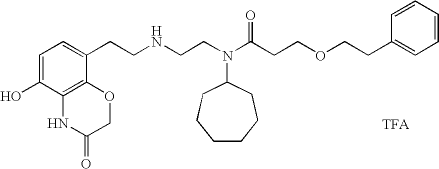

¹H NMR (399.825 MHz, CD₃OD) δ 7.26-7.12 (m, 5H), 6.72 (d, J=8.0 Hz, 1H), 6.49 (d, J=8.4 Hz, 1H), 4.62 (s, 2H), 3.91-3.82 (m, 1H), 3.72-3.64 (m, 4H), 3.50-3.46 (m, 2H), 3.17-3.12 (m, 2H), 3.10-3.07 (m, 2H), 2.92-2.87 (m, 2H), 2.85-2.80 (m, 2H), 2.64 (t, J=5.9 Hz, 2H), 1.80-1.44 (m, 12H).

### EXAMPLE 96

### N-Cycloheptyl-N-(2-(2-(5-hydroxy-3-oxo-3,4-dihydro-2H-benzo[b][1,4]oxazin-8-yl)ethylamino)ethyl)-3-(2-(pyridin-2-yl)ethoxy)propanamide trifluoroacetate

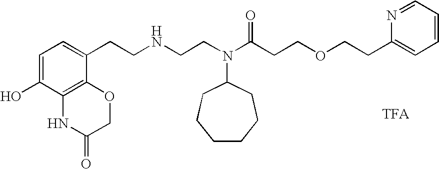

¹H NMR (399.825 MHz, CD₃OD) δ 8.68-8.65 (m, 1H), 8.36-8.31 (m, 1H), 7.86-7.82 (m, 1H), 7.78-7.73 (m, 1H), 6.71 (d, J=8.5 Hz, 1H), 6.46 (d, J=8.1 Hz, 1H), 4.60 (s, 2H), 3.85 (t, J=5.8 Hz, 2H), 3.83-3.75 (m, 1H), 3.71 (t, J=6.0 Hz, 2H), 3.50 (t, J=6.0 Hz, 2H), 3.23 (t, J=6.0 Hz, 2H), 3.18 (t, J=7.2 Hz, 2H), 3.12 (t, J=6.0 Hz, 2H), 2.90 (t, J'7.0 Hz, 2H), 2.67 (t, J=6.2 Hz, 2H), 1.80-1.42 (m, 12H).

### EXAMPLEs 97-100

Prepared by analogous procedures to Example 79, using 8-(2-aminoethyl)-5-hydroxy-2H-benzo[b][1,4]oxazin-3(4H)-one hydrochloride [Example 23, Step vii] in place of 5-(2-aminoethyl)-8-hydroxyquinolin-2(1H)-one hydrochloride. Further substitution of reagents was made as appropriate. Purification of the title compounds was achieved by a single reverse phase HPLC purification (eluting with a gradient of acetonitrile in 0.2% aqueous trifluoroacetic acid). Pure product-containing fractions were combined and concentrated to afford the title compounds as white solids.

### EXAMPLE 97

### N-(2-(2-(5-Hydroxy-3-oxo-3,4-dihydro-2H-benzo[b][1,4]oxazin-8-yl)ethylamino)ethyl)-N-isopropyl-3-phenethoxypropanamide trifluoroacetate

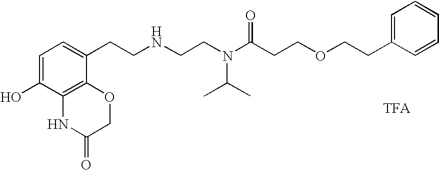

¹H NMR (399.825 MHz, CD₃OD) δ7.25-7.11 (m, 5H), 6.71 (d, J=8.2 Hz, 1H), 6.48 (d, J=8.2 Hz, 1H), 4.61 (s, 2H), 4.25-4.13 (m, 1H), 3.72-3.63 (m, 4H), 3.48 (t, J=5.6 Hz, 2H), 3.14 (t, J=7.2 Hz, 2H), 3.07 (t, J=5.6 Hz, 2H), 2.89 (t, J=7.2 Hz, 2H), 2.82 (t, J=6.8 Hz, 2H), 2.63 (t, J=6.0 Hz, 2H), 1.15 (d, J=6.7 Hz, 6H).

### EXAMPLE 98

### (R)—N-(2-(2-(5-Hydroxy-3-oxo-3,4-dihydro-2H-benzo[b][1,4]oxazin-8-yl)ethylamino)ethyl)-N-(3-methylbutan-2-yl)-3-phenethoxypropanamide trifluoroacetate

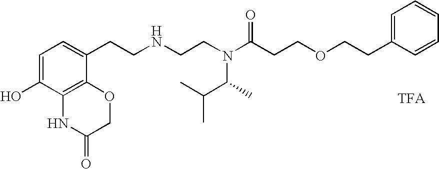

¹H NMR (399.825 MHz, CD₃OD) δ 7.26-7.11 (m, 5H), 6.72 (d, J=8.5 Hz, 1H), 6.48 (d, J=8.2 Hz, 1H), 4.61 (d, J=1.0 Hz, 2H), 3.72-3.54 (m, 6H), 3.36-2.99 (m, 5H), 2.89 (t, J=7.2 Hz, 2H), 2.82 (t, J=6.9 Hz, 2H), 2.74-2.65 (m, 1H), 2.59-2.50 (m, 1H), 1.76-1.65 (m, 1H), 1.17 (d, J=6.7 Hz, 3H), 0.96 (d, J=6.7 Hz, 3H), 0.83 (d, J=6.7 Hz, 3H).

### EXAMPLE 99

### N-Cyclopentyl-N-(2-(2-(5-hydroxy-3-oxo-3,4-dihydro-2H-benzo[b][1,4]oxazin-8-yl)ethylamino)ethyl)-3-phenethoxypropanamide trifluoroacetate

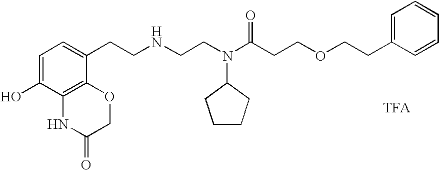

¹H NMR (399.825 MHz, CD₃OD) δ 7.26-7.12 (m, 5H), 6.71 (d, J=8.7 Hz, 1H), 6.48 (d, J=8.7 Hz, 1H), 4.61 (s, 2H), 4.27-4.21 (m, 1H), 3.72-3.63 (m, 4H), 3.50-3.45 (m, 2H), 3.17-3.05 (m, 4H), 2.89 (t, J=7.7 Hz, 2H), 2.82 (t, J=7.7 Hz, 2H), 2.66 (t, J=6.1 Hz, 2H), 1.91-1.80 (m, 2H), 1.76-1.56 (m, 4H), 1.47-1.38 (m, 2H).

### EXAMPLE 100

### N-(2-(2-(5-Hydroxy-3-oxo-3,4-dihydro-2H-benzo[b][1,4]oxazin-8-yl)ethylamino)ethyl)-N-neopentyl-3-phenethoxypropanamide trifluoroacetate

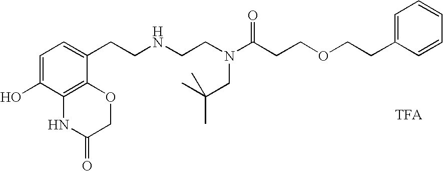

¹H NMR (399.825 MHz, CD₃OD) δ 7.28-7.11 (m, 5H), 6.73-6.69 (m, 1H), 6.51-6.46 (m, 1H), 4.61-4.52 (m, 2H), 3.84-3.43 (m, 6H), 3.22-3.19 (m, 2H), 3.13-3.02 (m, 4H), 2.90-2.77 (m, 4H), 2.64-2.45 (m, 2H), 0.97-0.87 (m, 9H).

### EXAMPLE 101

### 3-(3-Chlorophenethoxy)-N-(2-(diethylamino)ethyl)-N-(2-(2-(5-hydroxy-3-oxo-3,4-dihydro-2H-benzo[b][1,4]oxazin-8-yl)ethylamino)ethyl)propanamide bis(trifluoroacetate)

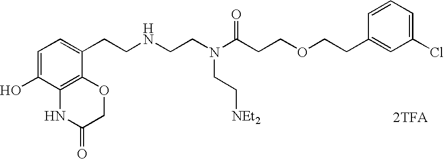

### Step i) 3-(3-Chlorophenethoxy)-N-(2-(diethylamino)ethyl)-N-(2-oxoethyl)propanamide

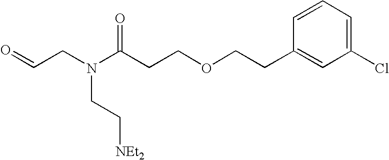

Trifluoroacetic acid (10 mL) was added to a solution of 3-(3-chlorophenethoxy)-N-(2-(diethylamino)ethyl)-N-(2,2-dimethoxyethyl)propanamide (0.86 g) [WO2007027133] in dichloromethane (10 mL). The solution was stirred for 1 h, then concentrated in vacuo and the residual yellow oil was redissolved in dichloromethane (4.2 mL) to give a 0.5 M solution.

MS (APCI+) 369 [M+H]⁺

### Step ii) 3-(3-Chlorophenethoxy)-N-(2-(diethylamino)ethyl)-N-(2-(2-(5-hydroxy-3-oxo-3,4-dihydro-2H-benzo[b][1,4]oxazin-8-yl)ethylamino)ethyl)propanamide bis(trifluoroacetate)

8-(2-Aminoethyl)-5-hydroxy-2H-benzo[b][1,4]oxazin-3(4H)-one hydrochloride (0.122 g) [Example 23, Step vii] and sodium bicarbonate (0.042 g) were combined in NMP (2 mL) and water (0.2 mL) and the mixture stirred for 20 minutes. 3-(3-Chlorophenethoxy)-N-(2-(diethylamino)ethyl)-N-(2-oxoethyl)propanamide (0.5 M in dichloromethane; 1 mL) [Step i] was added and stirring continued for a further 20 minutes, prior to addition of sodium triacetoxyborohydride (0.159 g). The mixture was stirred for 17 h, then diluted with saturated aqueous sodium bicarbonate solution (2 mL) and dichloromethane (5 mL). The mixture was separated using a 12 mL Biotage phase separation cartridge and the organic phase concentrated in vacuo. The residue was purified using reverse phase HPLC (eluting with a 10-30% gradient of acetonitrile in 0.2% aqueous trifluoroacetic acid). Pure product-containing fractions were combined and concentrated in vacuo to afford the title compound as a white solid (26 mg).

MS (APCI+) 561 [M+H]⁺

¹H NMR (399.825 MHz, CD₃OD) δ 7.27-7.10 (m, 4H), 7.25-7.11 (m, 4H), 6.73-6.69 (m, 1H), 6.50-6.46 (m, 1H), 4.59 (d, J=18.5 Hz, 2H), 3.77-3.63 (m, 8H), 3.26-3.14 (m, 6H), 2.94-2.87 (m, 2H), 2.86-2.80 (m, 2H), 2.65-2.59 (m, 2H), 1.32-1.26 (m, 6H).

### EXAMPLE 102

### 3-(3-chlorophenethoxy)-N-(2-(diethylamino)ethyl)-N-(2-(2-(8-hydroxy-2-oxo-1,2-dihydroquinolin-5-yl)ethylamino)ethyl)propanamide bis(trifluoroacetate)

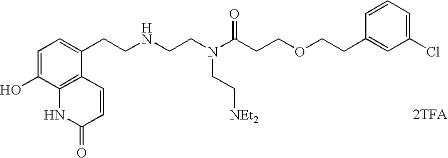

Prepared by an analogous procedure to Example 101, using 5-(2-aminoethyl)-8-hydroxyquinolin-2(1H)-one hydrochloride [J. Med. Chem. 1985, 28, 1803] in place of 8-(2-aminoethyl)-5-hydroxy-2H-benzo[b][1,4]oxazin-3(4H)-one hydrochloride in Step ii.

MS (APCI+) 559 [M+H]⁺

¹H NMR (399.825 MHz, CD₃OD) δ 8.16 (dd, J=9.6, 4.7 Hz, 1H), 7.25-7.08 (m, 4H), 7.03-6.93 (m, 2H), 6.66 (d, J=9.7 Hz, 1H), 3.81-3.63 (m, 8H), 3.26-3.17 (m, 12H), 2.82 (q, J=6.0 Hz, 2H), 2.64 (t, J=5.5 Hz, 2H), 1.33-1.26 (m, 6H).

### Biological Assays

Adrenergic β2 Mediated cAMP Production

### Cell Preparation

H292 cells were grown in 225 cm2 flasks incubator at 37° C., 5% CO₂ in RPMI medium containing, 10% (v/v) FBS (foetal bovine serum) and 2 mM L-glutamine.

### Experimental Method

Adherent H292 cells were removed from tissue culture flasks by treatment with Accutase™ cell detachment solution for 15 minutes. Flasks were incubated for 15 minutes in a humidified incubator at 37° C., 5% CO₂. Detached cells were re-suspended in RPMI media (containing 10% (v/v) FBS and 2 mM L-glutamine) at 1×10⁶ cells per mL. 10000 cells in 100 μL were added to each well of a tissue-culture-treated 96-well plate and the cells incubated overnight in a humidified incubator at 37° C., 5% CO₂. The culture media was removed and cells were washed twice with 100 μL assay buffer and replaced with 50 μL assay buffer (HBSS solution containing 10 mM HEPES pH7.4 and 5 mM glucose). Cells were rested at room temperature for 20 minutes after which time 25 μL of rolipram (1.2 mM made up in assay buffer containing 2.4% (v/v) dimethylsulphoxide) was added. Cells were incubated with rolipram for 10 minutes after which time test compounds were added and the cells were incubated for 60 minutes at room temperature. The final rolipram concentration in the assay was 300 μM and final vehicle concentration was 1.6% (v/v) dimethylsulphoxide. The reaction was stopped by removing supernatants, washing once with 100 μL assay buffer and replacing with 50 μL lysis buffer. The cell monolayer was frozen at −80° C. for 30 minutes (or overnight).

AlphaScreen™ cAMP Detection

The concentration of cAMP (cyclic adenosine monophosphate) in the cell lysate was determined using AlphaScreen™ methodology. The frozen cell plate was thawed for 20 minutes on a plate shaker then 10 μL of the cell lysate was transferred to a 96-well white plate. 40 μL of mixed AlphaScreen™ detection beads pre-incubated with biotinylated cAMP, was added to each well and the plate incubated at room temperature for 10 hours in the dark. The AlphaScreen™ signal was measured using an EnVision spectrophotometer (Perkin-Elmer Inc.) with the recommended manufacturer's settings. cAMP concentrations were determined by reference to a calibration curve determined in the same experiment using standard cAMP concentrations. Concentration response curves for agonists were constructed and data was fitted to a four parameter logistic equation to determine both the pEC₅₀ and Intrinsic Activity. Intrinsic Activity was expressed as a fraction relative to the maximum activity determined for formoterol in each experiment. Results for compounds of the invention are to be found in Table 1.

### Selectivity Assays

### Adrenergic α1D

### Membrane Preparation

Membranes were prepared from human embryonic kidney 293 (HEK93) cells expressing recombinant human α1_{D} receptor. These were diluted in Assay Buffer (50 mM HEPES, 1 mM EDTA, pH 7.4) to provide a final concentration of membranes that gave a clear window between maximum and minimum specific binding.

### Experimental Method

Assays were performed in U-bottomed 96-well polypropylene plates. 10 μL [³H]-prazosin (0.3 nM final concentration) and 10 mL of test compound (10× final concentration) were added to each test well. For each assay plate 8 replicates were obtained for [³H]-prazosin binding in the presence of 10 μL vehicle (10% (v/v) DMSO in Assay Buffer; defining maximum binding) or 10 μL BMY7378 (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-prazosin binding) were determined using serial dilutions typically in the range 0.1 nM to 10 μM. Data was fitted to a four parameter logistic equation to determine the compound potency, which was expressed as pIC50 (negative log molar concentration inducing 50% inhibition of [³H]-prazosin binding). Results are shown in Table 1 below.

### Adrenergic β1

### Membrane Preparation

Membranes containing recombinant human adrenergic beta 1 receptors were obtained from Euroscreen. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 10 μL [¹²⁵I]-Iodocyanopindolol (0.036 nM final concentration) and 10 μL of test compound (10× final concentration) were added to each test well. For each assay plate 8 replicates were obtained for [¹²⁵I]-Iodocyanopindolol binding in the presence of 10 μL vehicle (10% (v/v) DMSO in Assay Buffer; defining maximum binding) or 10 μL Propranolol (10 μM final concentration; defining non-specific binding (NSB)). Membranes were then added to achieve a final volume of 100 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-O (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [¹²⁵I]-Iodocyanopindolol binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [¹²⁵I]-Iodocyanopindolol binding). Results are shown in Table 1 below.

### Dopamine D2

### Membrane Preparation

Membranes containing recombinant human Dopamine Subtype D2s receptors were obtained from Perkin Elmer. These were diluted in Assay Buffer (50 mM HEPES, 1 mM EDTA, 120 mM NaCl, 0.1% gelatin, pH 7.4) to provide a final concentration of membranes that gave a clear window between maximum and minimum specific binding.

### Experimental Method

Assays were performed in U-bottomed 96-well polypropylene plates. 30 μL [³H]-spiperone (0.16 nM final concentration) and 30 μL of test compound (10× final concentration) were added to each test well. For each assay plate 8 replicates were obtained for [³H]-spiperone binding in the presence of 30 μL vehicle (10% (v/v) DMSO in Assay Buffer; defining maximum binding) or 30 μL Haloperidol (10 μM final concentration; defining non-specific binding (NSB)). Membranes were then added to achieve a final volume of 300 μL. The plates were incubated for 2 hours at room temperature and then filtered onto PEI coated GF/B filter plates, pre-soaked for 1 hour in Assay Buffer, using a 96-well plate Tomtec cell harvester. Five washes with 250 μL wash buffer (50 mM HEPES, 1 mM EDTA, 120 mM NaCl, pH 7.4) were performed at 4° C. to remove unbound radioactivity. The plates were dried then sealed from underneath using Packard plate sealers and MicroScint-0 (50 μL) was added to each well. The plates were sealed (TopSeal A) and filter-bound radioactivity was measured with a scintillation counter (TopCount, Packard BioScience) using a 3-minute counting protocol.

Total specific binding (B₀) was determined by subtracting the mean NSB from the mean maximum binding. NSB values were also subtracted from values from all other wells. These data were expressed as percent of B₀. Compound concentration-effect curves (inhibition of [³H]-spiperone binding) were determined using serial dilutions typically in the range 0.1 nM to 10 μM. Data was fitted to a four parameter logistic equation to determine the compound potency, which was expressed as pIC₅₀ (negative log molar concentration inducing 50% inhibition of [³H]-spiperone binding).

The results obtained for a representative selection of the compounds of the Examples are shown in Table 1 below.

TABLE 1

| | | | α1 bind | β1 bind | D2 bind |
|---|---|---|---|---|---|
| Example No. | β2 pEC50 | β2 Int Act | pIC50 | pIC50 | pIC50 |
| 1 | 6.4 | 0.9 | | | |
| 2 | 7.9 | 0.93 | 6.1 | <5.2 | <5.2 |
| 3 | 8.1 | 0.82 | 7.1 | 5.9 | 6.7 |
| 4 | 7.7 | 0.79 | 7.1 | 5.5 | 7.1 |
| 5 | 8.1 | 0.82 | 6.8 | 5.3 | 6.3 |
| 6 | 7.5 | 0.65 | 7.3 | 5.3 | 6.6 |
| 7 | 8 | 0.76 | 6.9 | 5.2 | 6.5 |
| 8 | 8.2 | 0.79 | 7 | 5.2 | 6.4 |
| 9 | 7.4 | 0.91 | | | |
| 10 | 7.7 | 0.85 | 6.5 | <5.1 | |
| 11 | 7.1 | 0.65 | 6.5 | <5.0 | 6.3 |
| 12 | 7.3 | 0.67 | 6.5 | 5.3 | 6.9 |
| 13 | 8.1 | 0.86 | 7.1 | <5.0 | 7.3 |
| 14 | 7.7 | 0.88 | 6.3 | <5.0 | 6.6 |
| 15 | 8.1 | 0.88 | 6.3 | <5.0 | 5.9 |
| 16 | 7.7 | 0.85 | 6.5 | <5.0 | |
| 17 | 8.4 | 0.93 | 6.6 | 4.9 | 5.9 |
| 18 | 7.9 | 0.95 | 6.4 | <5.0 | 5.5 |
| 19 | 8 | 0.87 | 6.7 | 5 | 5.9 |
| 20 | 8.1 | 0.78 | 6.8 | 5.3 | 6.4 |
| 21 | 7.5 | 0.83 | 7.1 | <5.0 | 5.9 |
| 22 | 7.7 | 1.01 | 6.7 | 5.6 | 6.3 |
| 23 | 9.2 | 0.79 | 6.9 | 6.2 | 6.5 |
| 24 | 9 | 0.85 | 7.3 | 6.2 | 6.8 |
| 25 | 9 | 0.9 | 7.2 | 5.6 | 7 |
| 26 | 8.6 | 0.74 | 7.1 | 5.7 | 7.5 |
| 27 | 8.7 | 0.89 | 6.9 | 5.5 | 6.6 |
| 28 | 8.6 | 0.86 | 7.4 | 5.2 | 7.1 |
| 29 | 8.3 | 0.91 | 6.9 | 5.5 | 7 |
| 30 | 8.4 | 0.92 | 6.6 | 5.6 | 7.3 |
| 31 | 8.2 | 0.94 | 6.8 | 5.5 | 7 |
| 32 | 8.5 | 1.03 | 6.6 | 5.7 | 6.7 |
| 33 | 8.8 | 0.9 | 7.1 | 5.6 | |
| 34 | 8.8 | 0.9 | 7.2 | 6 | |
| 35 | 8.8 | 0.87 | 6.4 | <5.0 | 6.6 |
| 36 | 8.9 | 0.91 | 6.4 | 5.5 | 6.2 |
| 37 | 8.7 | 0.89 | 6.8 | 6 | 7 |
| 38 | 8.9 | 0.98 | 6.8 | <5.3 | 6.9 |
| 39 | 8.8 | 0.98 | 6.3 | 5.9 | 7 |
| 40 | 6.6 | 0.96 | 7.5 | <5.1 | 6.4 |
| 41 | 8.5 | 0.95 | 6.6 | <5.1 | 6.7 |
| 42 | 8.7 | 0.9 | 6.7 | 5.5 | 6.5 |
| 43 | 7.5 | 0.97 | <5.0 | <5.0 | <5.0 |
| 44 | 8.7 | 0.89 | 7.3 | 5.9 | 7.1 |
| 45 | 8.3 | 0.89 | 6.6 | 5.9 | 7.1 |
| 46 | 8.9 | 0.89 | 7.1 | 5.8 | 7.8 |
| 47 | 8.8 | 0.89 | 7.1 | 6.2 | 7.3 |
| 48 | 8.7 | 0.91 | 6.9 | 6.1 | 7.1 |
| 49 | 8.6 | 1.01 | 6.5 | 6 | 7.2 |
| 50 | 8.9 | 0.89 | 6.7 | 5.9 | 7.5 |
| 51 | 8.8 | 0.94 | 6.5 | 5.7 | 7.4 |
| 52 | 8 | 0.95 | 6.7 | 5.8 | 7.2 |
| 53 | 8.2 | 0.99 | 6.7 | 5.6 | 7.4 |
| 54 | 8.3 | 0.96 | 7.1 | 5.7 | 7.5 |
| 55 | 7.5 | 0.92 | 6.6 | 6 | 7.3 |
| 56 | 8.1 | 0.91 | 7.5 | 5.7 | 7.5 |
| 57 | 8.1 | 0.91 | 7 | 5.8 | 7.8 |
| 58 | 8.1 | 0.91 | 7 | 5.7 | 7.4 |
| 59 | 8.4 | 0.87 | 6.7 | 5.7 | 7 |
| 60 | 8.4 | 0.92 | 7 | 5.6 | 7.3 |
| 61 | 8.5 | 0.84 | 7.7 | 6.3 | 7 |
| 62 | 8.4 | 0.86 | 7.4 | 6.6 | 7.2 |
| 63 | 8.7 | 0.79 | 7 | 6.6 | 7.4 |
| 64 | 9.2 | 0.88 | 7.2 | 5.9 | 7.2 |
| 65 | 8.8 | 0.93 | 6.4 | 5.6 | 6.9 |
| 66 | 8.6 | 0.99 | 6.4 | 5.8 | 7.4 |
| 67 | 8.5 | 0.87 | 6.7 | 6 | 7 |
| 68 | 8.4 | 0.93 | 6.8 | 6.4 | 7 |
| 69 | 8.8 | 0.88 | 6.7 | 6.1 | 6.8 |
| 70 | 8.4 | 0.97 | 5.8 | 5.4 | 6 |
| 71 | 8 | 0.85 | 6.2 | 5.3 | 5.4 |
| 72 | 8.3 | 0.92 | 6.3 | 5.2 | 5.4 |
| 73 | 8.4 | 0.94 | 6.2 | 5.3 | 5.3 |
| 74 | 8.7 | 0.97 | 6.1 | 5.6 | 5.7 |
| 75 | 8.6 | 0.96 | 6.2 | 5.9 | 5.7 |
| 76 | 8.8 | 1.1 | 6 | 5.7 | 5.6 |
| 77 | 8.7 | 0.97 | 6.1 | 5.5 | 5.7 |
| 78 | 7.7 | 1.01 | 6.1 | 5.1 | 5.2 |
| 79 | 7.3 | 0.92 | 6.2 | <5.0 | <5.0 |
| 80 | 7.6 | 1.04 | 6.3 | <5.0 | 5.6 |

### Onset Assay

Dunkin-Hartley guinea-pigs (between 200 g and 300 g on delivery) were supplied by a designated breeding establishment. The guinea-pigs were killed by cervical dislocation and the trachea removed. The adherent connective tissue was removed and each trachea cut into four rings. The tissue rings were then attached to an isometric transducer. The tissues were washed and a force of 1 g was applied to each ring. In all experiments a paired curve design was used. A priming dose of 1 μM methacholine was applied to the tissues. The tissues were then washed (three times, one minute between washes), the resting tension of 1 g was reapplied and the tissues were allowed to rest for 1 hour to equilibrate. Tissues were then contracted with 1 μM methacholine and once a steady response was obtained a cumulative concentration response curve to isoprenaline (10⁻⁹ M−10⁻⁵ M) was constructed. The tissues were then washed (three times, one minute between washes) and left to rest for an hour. At the end of the resting period the tissues were contracted with 1 μM methacholine and a p[A]₅₀ concentration of test compound added. Once the tissue had reached maximum relaxation, a 30×p[A]₅₀ concentration of test compound was added. Once the tissue response had reached a plateau, 30 μM sotalol was added to the bath to confirm that the relaxation was β₂ mediated

Data were collected using the ADInstruments chart5 for windows software, which measured the maximum tension generated at each concentration of agonist.

For each concentration of the isoprenaline cumulative concentration curve, the response was calculated as % relaxation of the methacholine-induced contraction. A curve was plotted of log₁₀[agonist] (M) versus percentage inhibition of the methacholine-induced contraction. These data were then fitted to a non-linear regression curve fit. For each experiment, E/[A] curve data were fitted using a 4-parameter logistic function of the form:

$$E = β + \frac{( β - α ) \cdot {[ A ]}^{m}}{{[ A ]}^{m} + {[ A ]}_{50}^{m}}$$

E and [A] are the pharmacological effect (% relaxation) and concentration of the agonist respectively; α, β, [A]₅₀ and m are the asymptote, baseline, location and slope parameters, respectively. The p[A]₅₀ and IA of each isoprenaline curve was determined from this fit, to determine if the tissue was viable for generating an onset time for the test compounds.

For each p[A]₅₀ concentration of the test compound, the response was calculated as % relaxation of the methacholine-induced contraction. The results were plotted % relaxation against time and the time taken to reach a 90% relaxation value was calculated and recorded.

The addition of a 30×p[A]₅₀ concentration enabled determination of the maximum compound effect within the individual tissue. Hence, the % of the maximum compound effect at the p[A]₅₀ concentration was calculated and recorded.

### Pharmacokinetics in the Rat

A dose solution of the test compound was prepared using a suitable dose vehicle. The concentration of the compound in the dose solution was assayed by diluting an aliquot to a nominal concentration of 50 μg·ml⁻¹ and calibrating against duplicate injections of a standard solution and a QC standard at this concentration. Compounds were administered intravenously as a bolus into a caudal vein to groups of three 250-350 g rats (approximately 1 ml·kg⁻¹). For the oral dose, a separate group of 2 or 3 animals were dosed by oral gavage (3 ml·kg⁻¹). Delivered doses were estimated by weight loss. Food was not usually withdrawn from animals prior to dosing, although this effect was investigated if necessary.

Blood samples (0.25 ml) were taken into 1 ml syringes from the caudal vein, transferred to EDTA tubes and plasma was prepared by centrifugation (5 min at 13000 rpm) soon after sample collection, before storage at −20° C. Typical sampling times were 2, 4, 8, 15, 30, 60, 120, 180, 240, 300 (min) or until the terminal t1/2 was accurately described.

The concentration of the analyte(s) were determined in plasma by quantitative mass spectrometry. Standard and quality control stock solutions were prepared at a concentration 1 mg/ml in methanol. A range of standard and QC stocks produced by serial dilution were added to control rat plasma (50 μl). The range of concentrations covered the range of levels of analyte present in the rat samples. Standards, QCs and samples underwent liquid extraction using 50 μl of organic solvent and 100 μl of organic solvent containing an internal standard, chosen to closely resemble the analyte. The samples were then mixed by repeated inversion, stored at −20° C. for at least 1 h, and centrifuged at 3500 rpm in a centrifuge for 20 minutes. Aliquots (120 μl) of each sample were transferred for analysis using LC-MSMS. Standard and quality control samples covering the range of concentrations found in the test samples were within 25% of the nominal concentration.

Pharmacokinetic data analysis was achieved using WinNonlin. A standard non-compartmental analysis was used to estimate the parameters such as Tmax, Cmax, Lambda_z, t1/2_Lambda_z, AUCall, AUCINF(observed), Cl(observed), Vss(observed).

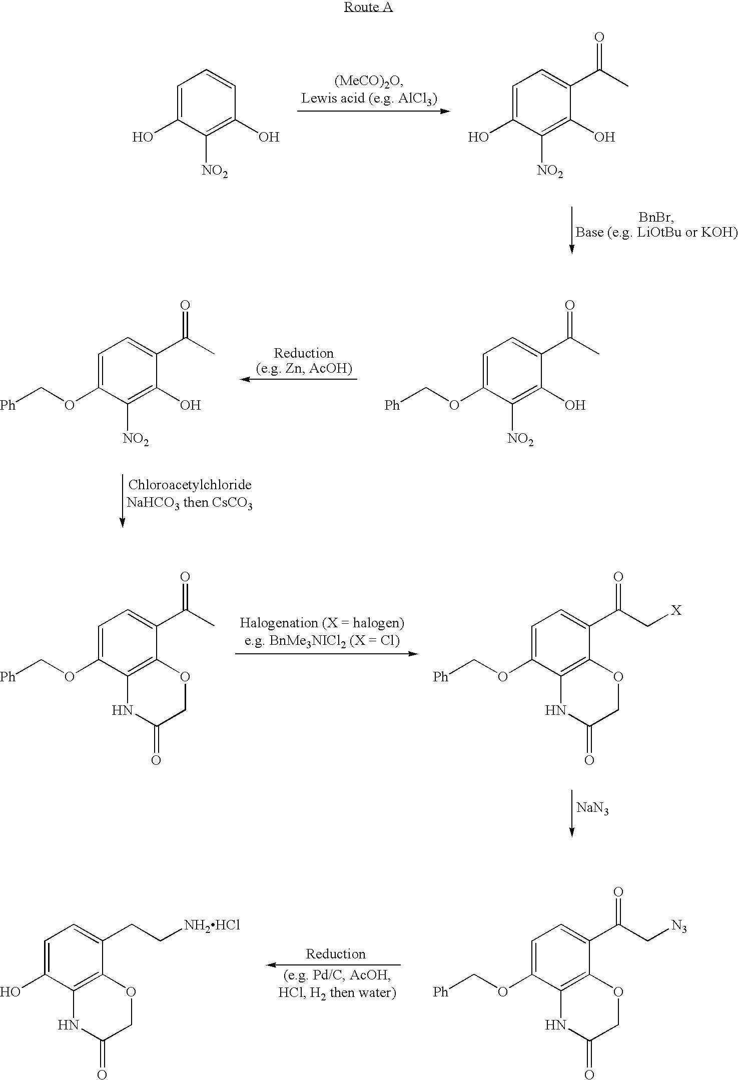

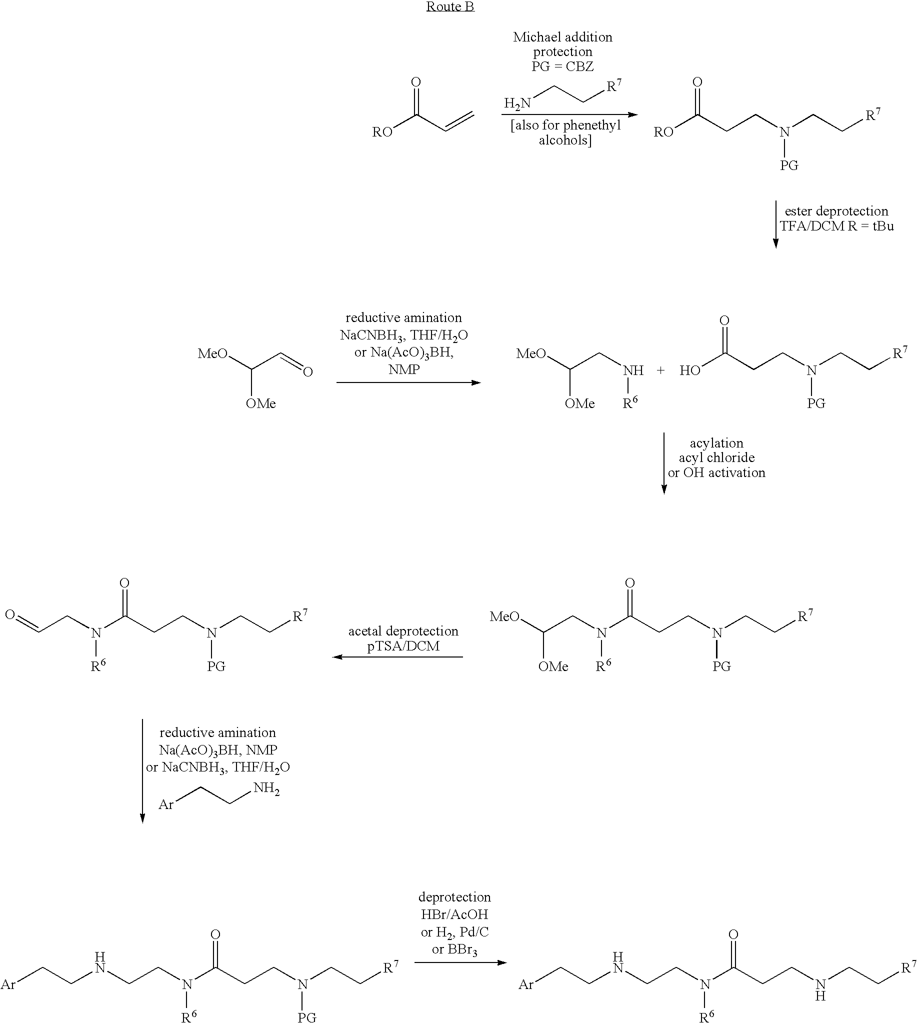

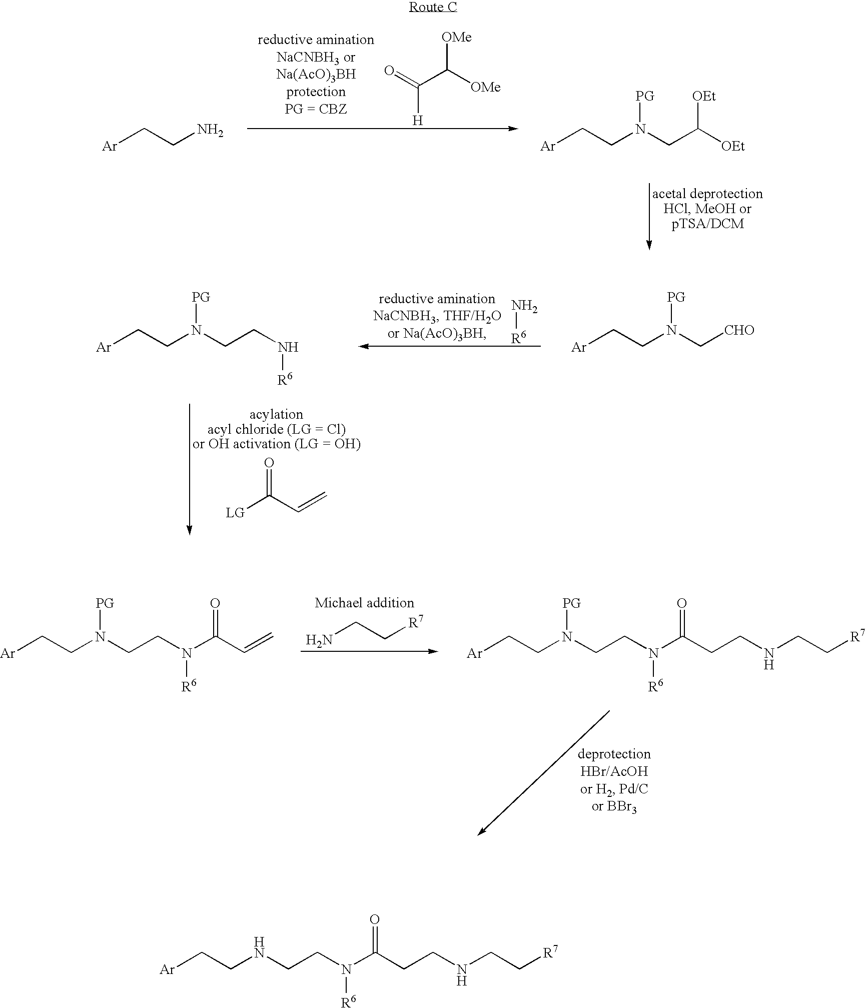

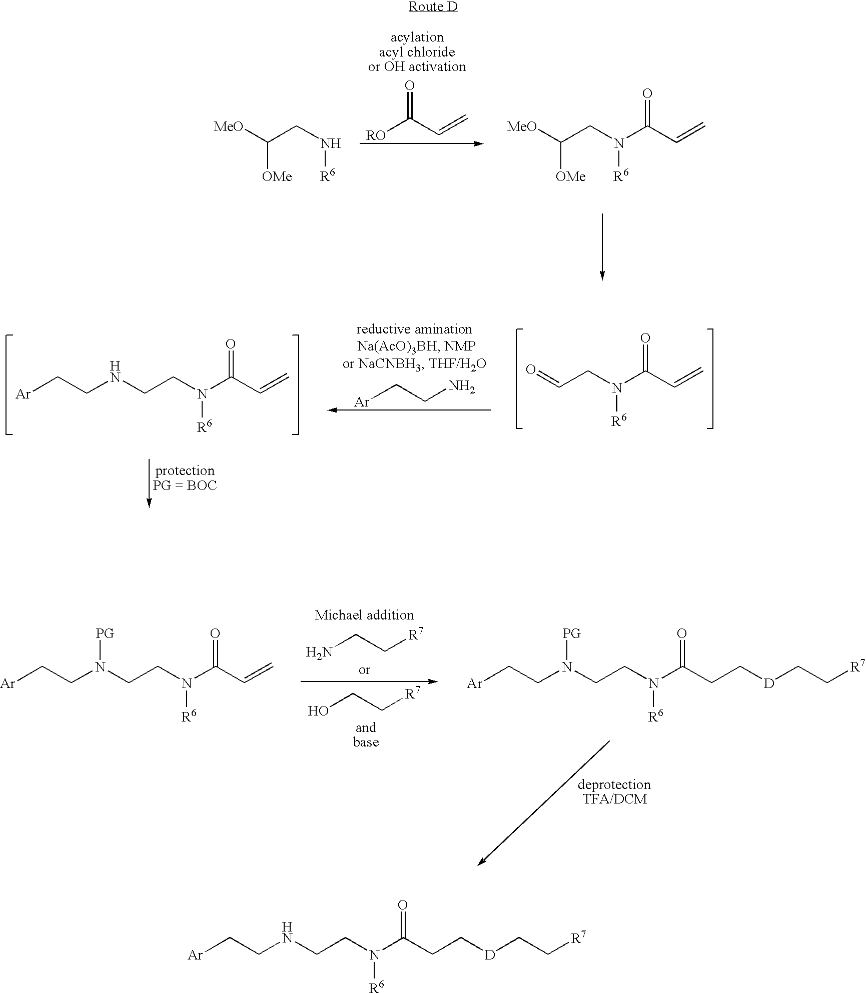

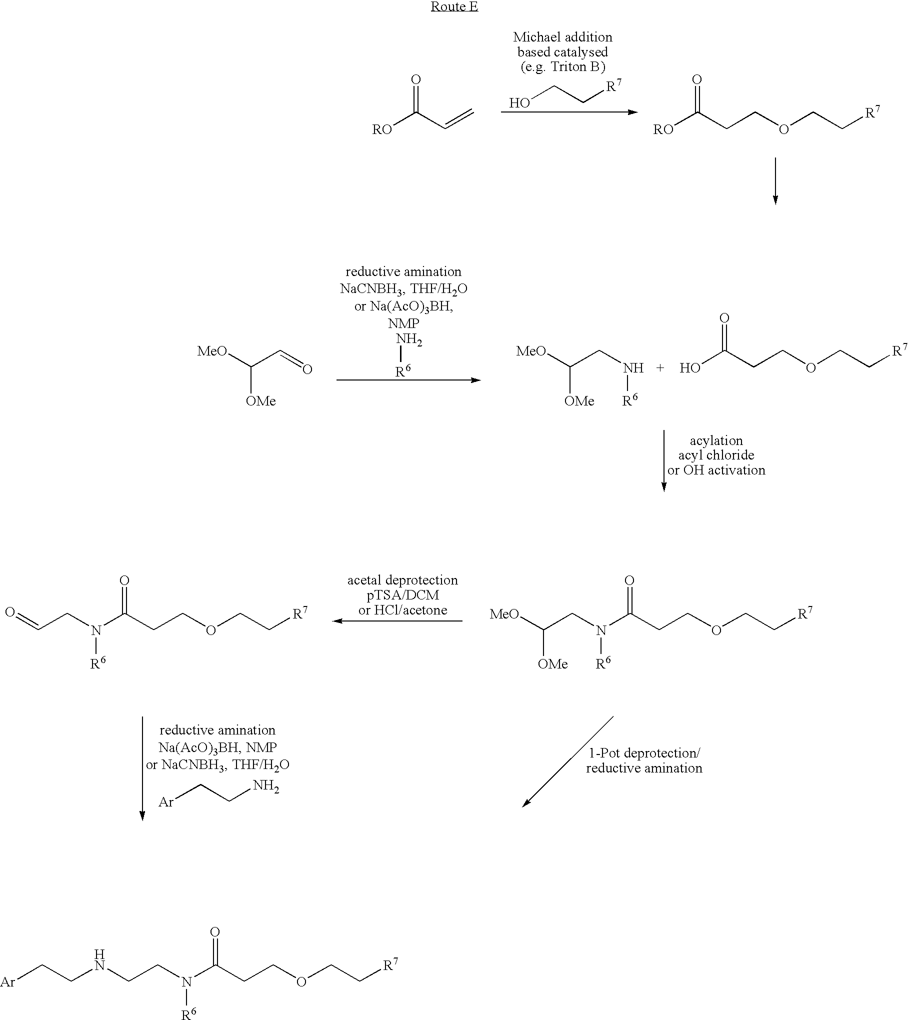

1. A compound or a pharmaceutically acceptable salt thereof, wherein:
the compound corresponds to formula (I): 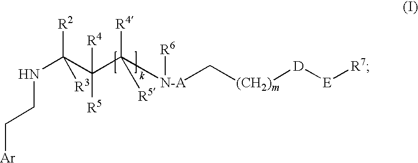
Ar is: 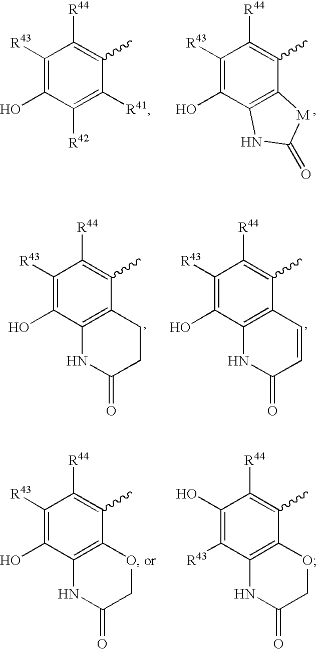
each of R², R³, R⁴, R⁵, R^{4′} and R^{5′} is, independently, hydrogen or C₁₋₆ alkyl;
A is CH₂, C(O), or S(O)₂;
D is oxygen, sulphur, or NR⁸;
E is a bond, CR⁶³R⁶⁴, CR⁶³R⁶⁴CR⁶⁵R⁶⁶, CR⁶³R⁶⁴CR⁶⁵R⁶⁶CR⁶⁷R⁶⁸, or CR⁶³R⁶⁴CR⁶⁵R⁶⁶CR⁶⁷R⁶⁸CR⁶⁹R⁷⁰;
when D is sulphur or NR⁸, R⁶³ and R⁶⁴ are, independently, hydrogen or C₁₋₄ alkyl;
when D is oxygen, R⁶³ and R⁶⁴ are, independently, fluoro, hydrogen, or C₁₋₄ alkyl;
R⁶⁵, R⁶⁶, R⁶⁷, R⁶⁸, R⁶⁹, and R⁷⁰ are, independently, hydrogen, fluoro, or C₁₋₄ alkyl;
k is zero or 1;
m is zero, 1, 2, or 3;
R⁶ is:
—(X)ₚ—Y—(Z)_{q}—R¹⁰, or
an α- or β-branched C₃₋₁₂ alkyl, wherein:
the α- or β-branched C₃₋₁₂ alkyl is optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁵⁸R⁵⁹, OC(O)(C₁₋₆ alkyl), C₃₋₁₂ cycloalkyl, or R⁶⁰;
X and Z are, independently, a C₁₋₆ alkylene group, wherein:
the C₁₋₆ alkylene group is optionally substituted with halogen, trifluoromethyl, NH₂, (di)-C₁₋₆ alkylamino, (di)-C₁₋₆ alkylaminocarbonyl, C₁₋₆ alkylcarbonylamino, —SO₂NH₂, or (di)-C₁₋₆ alkylaminosulphonyl;
p and q are, independently, zero or 1;
when p is 1, Y is a bond, oxygen, sulphur, CH₂, C(O), or NR⁹;
when p is zero, Y is a bond, oxygen, CH₂, C(O), or NR⁹;
M is C(O), NR⁵¹, or CR⁵²R⁵³ except:
M is not CH₂ or NR⁵¹ when R⁶ is C₁₋₆ alkyl;
R⁸ is hydrogen or C₁₋₆ alkyl;
R⁹ is hydrogen or C₁₋₆ alkyl;
R¹⁰ is hydrogen or a saturated or unsaturated 3- to 12-membered ring system, wherein:
the saturated or unsaturated 3- to 12-membered ring system optionally comprises a ring heteroatom selected from nitrogen, oxygen, and sulphur, and
the saturated or unsaturated 3- to 12-membered ring system is optionally substituted by halogen, trifluoromethyl, cyano, carboxyl, hydroxy, —S(O)ᵣR¹⁵, —NR¹⁶S(O)ₛR¹⁷—C(O)NR¹⁸R¹⁹, —NHC(O)R²⁰, C₁₋₆ alkyl, C₁₋₆ alkoxy, C₁₋₆ alkylcarbonyl, C₁₋₆ alkoxycarbonyl, or a saturated or unsaturated 4- to 7-membered monocyclic ring system, wherein:
the saturated or unsaturated 4- to 7-membered monocyclic ring system optionally comprises at least one ring heteroatom selected from nitrogen, oxygen, and sulphur, and
the saturated or unsaturated 4- to 7-membered monocyclic ring system is optionally substituted by halogen, trifluoromethyl, hydroxy, —NR²¹S(O)ₜR²², —NHC(O)R²³, or C₁₋₆ alkoxy;
R⁷ is a 5- to 14-membered aromatic or heteroaromatic ring system, wherein the 5- to 14-membered aromatic or heteroaromatic ring system is optionally substituted by:
halogen,
trifluoromethyl,
hydroxy,
carboxyl,
C₁₋₆ alkyl, wherein:
the C₁₋₆ alkyl is optionally substituted by halogen or —NR²⁴R²⁵,
C₁₋₆ alkoxy, wherein:
the C₁₋₆ alkoxy is optionally substituted by halogen or —NR²⁶R²⁷,
C₃₋₆ cycloalkyl,
C₂₋₄ alkenyl,
C₂₋₄ alkynyl,
cyano,
C₁₋₆ alkoxycarbonyl,
—NR²⁸R²⁹,
C₁₋₆ alkylcarbonylamino,
OC(O)(C₁₋₆ alkyl),
C₁₋₆ alkylthio,
C₁₋₆ alkylS(O),
C₁₋₆ alkylS(O)₂,
C₁₋₆ alkylsulphonylamino,
phenylsulphonylamino,
—C(O)NHR³⁰,
—SO₂NHR³³,
C₀₋₆ alkyl-R³⁴, or
a phenyl or 5- to 6-membered heteroaromatic ring, wherein:
each phenyl and 5- to 6-membered heteroaromatic ring is optionally substituted by halogen, trifluoromethyl, hydroxy, C₁₋₆ alkyl, C₁₋₆ alkoxy, or —NR³⁵R³⁶;
R²⁴, R²⁵, R²⁶, R²⁷, R²⁸, R²⁹, R⁵⁸, and R⁵⁹ are, independently, hydrogen or C₁₋₆ alkyl;
R³⁰ is hydrogen, C₁₋₆ alkyl, phenyl-C₀₋₆ alkyl, or C₂₋₆ alkylene-NR³¹R³²;
as to R³¹ and R³²,
R³¹ and R³² are, independently, hydrogen or C₁-C₆ alkyl, or
R³¹ and R³², together with the nitrogen atom to which they are attached, form a 4- to 6-membered saturated heterocyclic ring optionally comprising a further ring heteroatom selected from nitrogen and oxygen;
R³³ is hydrogen, C₁₋₆ alkyl, phenyl-C₀₋₆ alkyl, or C₂₋₆ alkylene-NR³⁷R³⁸;
R³⁴ is a saturated, 5- or 6-membered nitrogen-containing ring;
R³⁵ and R³⁶ are, independently, hydrogen or C₁₋₆ alkyl;
as to R³⁷ and R³⁸,
R³⁷ and R³⁸ are, independently, hydrogen or C₁₋₆ alkyl, or
R³⁷ and R³⁸, together with the nitrogen atom to which they are attached, form a 4- to 6-membered saturated heterocyclic ring optionally comprising a further ring heteroatom selected from nitrogen and oxygen;
R⁶⁰ is a 4- to 7-membered heterocyclyl, wherein:
the heterocyclyl comprises a ring nitrogen, oxygen, or sulphur, wherein:
the nitrogen is optionally substituted by C(O)(C₁₋₆ alkyl);
the heterocyclyl ring is optionally substituted by C₁₋₆ alkyl, and ring carbon atoms of the heterocyclyl which are not adjacent to a ring heteroatom are optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁶¹R⁶², OC(O)(C₁₋₆ alkyl, or C₃₋₁₂ cycloalkyl;
R⁴¹, R⁴³ and R⁴⁴ are, independently, hydrogen, halogen, trifluoromethyl, cyano, carboxyl, hydroxy, —S(O)_{V}R⁴⁵, —NR⁴⁶S(O)_{W}R⁴⁷, —C(O)NR⁴⁸R⁴⁹, —NR⁵⁵C(O)R⁵⁰, C₁₋₆ alkyl, C₁₋₆ alkoxy, C₁₋₆ alkylcarbonyl, or C₁₋₆ alkoxycarbonyl;
R⁴² is hydrogen, halogen, trifluoromethyl, cyano, carboxyl, hydroxy, —S(O)_{V}R⁴⁵, —NR⁴⁶S(O)_{W}R⁴⁷, —C(O)NR⁴⁸R⁴⁹, —NR⁵⁵C(O)R⁵⁰, C₁₋₆ alkyl, C₁₋₆ alkoxy, C₁₋₆ alkylcarbonyl, C₁₋₆ alkoxycarbonyl, CH₂OH, NHC(O)H, NHS(O)₂R⁵⁴, or NHS(O)₂NR⁵⁶R⁵⁷;
R¹⁵, R¹⁷, R²², R⁴⁵, R⁴⁷, and R⁵⁴ are, independently C₁₋₆ alkyl; and
R¹⁶, R¹⁸, R¹⁹, R²⁰, R²¹, R²³, R⁴⁶, R⁴⁸, R⁴⁹, R⁵⁰, R⁵¹, R⁵², R⁵³, R⁵⁵, R⁵⁶, R⁵⁷, R⁶¹ and R⁶² are, independently, C₁₋₆ alkyl or can also be hydrogen;
r and v are, independently, zero, 1, or 2; and
s, t, and w are all 2.

2. A compound of formula (I) or a pharmaceutically acceptable salt thereof as claimed in claim 1, wherein Ar is:

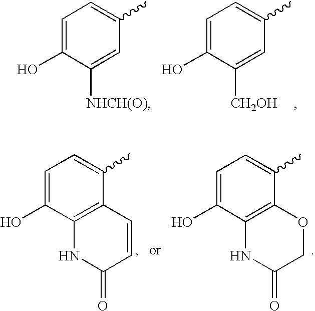

3. A compound of formula (I) or a pharmaceutically acceptable salt thereof as claimed in claim 1, wherein A is C(O).

4. A compound of formula (I) or a pharmaceutically acceptable salt thereof as claimed in claim 1, wherein k is zero.

5. A compound of formula (I) or a pharmaceutically acceptable salt thereof as claimed in claim 1, wherein m is 1.

6. A compound of formula (I) or a pharmaceutically acceptable salt thereof as claimed in claim 1, wherein D is oxygen, NH, or N(C₁₋₄ alkyl).

7. A compound of formula (I) or a pharmaceutically acceptable salt thereof as claimed in claim 1, wherein E is CH₂CH₂.

8. A compound of formula (I) or a pharmaceutically acceptable salt thereof as claimed in claim 1, wherein R², R³, R⁴, and R⁵, and, if present, R^{4′} and R^{5′}, are all hydrogen.

9. A compound of formula (I) or a pharmaceutically acceptable salt thereof as claimed in claim 1, wherein R⁶ is an α- or β-branched C₃₋₁₂ alkyl, wherein:
the C₃₋₁₂ alkyl is optionally substituted by one or more substituents selected from halogen, C₁₋₆ alkoxy, C₁₋₆ alkylthio, C₁₋₆ alkylS(O), C₁₋₆ alkylS(O)₂, C₁₋₆ haloalkoxy, hydroxy, and OC(O)(C₁₋₆ alkyl).

10. A compound of formula (I) or a pharmaceutically acceptable salt thereof as claimed in claim 1, wherein:
R⁶ is —(X)ₚ—Y—(Z)_{q}—R¹⁰;
p and q are both zero;
Y is a bond;
R¹⁰ is a saturated or unsaturated 3- to 12-membered ring system, wherein:
the saturated or unsaturated 3- to 12-membered ring system optionally comprises a ring heteroatom selected from nitrogen, oxygen, and sulphur, and
the saturated or unsaturated 3- to 12-membered ring system is optionally substituted by halogen, trifluoromethyl, cyano, carboxyl, hydroxy, —S(O)ᵣR¹⁵, —NR¹⁶S(O)ₛR¹⁷, —C(O)NR¹⁸R¹⁹, —NHC(O)R²⁰, C₁₋₆ alkyl, C₁₋₆ alkoxy, C₁₋₆ alkylcarbonyl, C₁₋₆ alkoxycarbonyl, or a saturated or unsaturated 4- to 7-membered monocyclic ring system, wherein:
the saturated or unsaturated 4- to 7-membered monocyclic ring system optionally comprises at least one ring heteroatom selected from nitrogen, oxygen, and sulphur, and
the saturated or unsaturated 4- to 7-membered monocyclic ring system is optionally substituted by halogen, trifluoromethyl, hydroxy, —NR²¹S(O)ₜR²², —NHC(O)R²³, or C₁₋₆ alkoxy; and
r is 2.

11. A compound of formula (I) or a pharmaceutically acceptable salt thereof as claimed in claim 1, wherein:
R⁷ is a 6- to 10-membered aromatic or heteroaromatic ring system, wherein:
the 6- to 10-membered aromatic or heteroaromatic ring system is optionally substituted by one or more substituents independently selected from:
halogen,
trifluoromethyl,
hydroxyl,
carboxyl,
C₁₋₄ alkyl, wherein:
the C₁₋₄ alkyl is optionally substituted by —NR²⁴R²⁵,
C₁₋₄ alkoxy, wherein:
the C₁₋₄ alkoxy is optionally substituted by —NR²⁶R²⁷,
C₁₋₄ alkoxycarbonyl,
—NR²⁸R²⁹,
C₁₋₄ alkylcarbonylamino,
C₁₋₄ alkylsulphonylamino,
—C(O)NHR³⁰, and
—SO₂NHR³³; and
R³⁰ and R³³ are, independently, hydrogen or C₁₋₆ alkyl.

12. A compound or a pharmaceutically acceptable salt thereof, wherein the compound is:
N-Butyl-N-(2-(2-(8-hydroxy-2-oxo-1,2-dihydroquinolin-5-yl)ethylamino)ethyl)-3-phenethoxypropanamide;
N-Cyclohexyl-N-(2-(2-(8-hydroxy-2-oxo-1,2-dihydroquinolin-5-yl)ethylamino)ethyl)-3-phenethoxypropanamide;
3-(3-Chlorophenethylamino)-N-cycloheptyl-N-(2-(2-(8-hydroxy-2-oxo-1,2-dihydroquinolin-5-yl)ethylamino)ethyl)propanamide;
3-(4-Chlorophenethylamino)-N-cycloheptyl-N-(2-(2-(8-hydroxy-2-oxo-1,2-dihydroquinolin-5-yl)ethylamino)ethyl)propanamide;
N-Cycloheptyl-3-(3-fluorophenethylamino)-N-(2-(2-(8-hydroxy-2-oxo-1,2-dihydroquinolin-5-yl)ethylamino)ethyl)propanamide;
N-Cycloheptyl-3-(4-fluorophenethylamino)-N-(2-(2-(8-hydroxy-2-oxo-1,2-dihydroquinolin-5-yl)ethylamino)ethyl)propanamide;
N-Cycloheptyl-N-(2-(2-(8-hydroxy-2-oxo-1,2-dihydroquinolin-5-yl)ethylamino)ethyl)-3-(phenethylamino)propanamide;
N-Cycloheptyl-N-(2-(2-(8-hydroxy-2-oxo-1,2-dihydroquinolin-5-yl)ethylamino)ethyl)-3-(3-methylphenethylamino)propanamide;
(S)—N-Cycloheptyl-N-(2-(2-(8-hydroxy-2-oxo-1,2-dihydroquinolin-5-yl)ethylamino)ethyl)-3-(2-phenylpropylamino)propanamide;
N-Cycloheptyl-3-(3,5-difluorophenethylamino)-N-(2-(2-(8-hydroxy-2-oxo-1,2-dihydroquinolin-5-yl)ethylamino)ethyl)propanamide;
N-Cycloheptyl-3-(3-ethoxyphenethylamino)-N-(2-(2-(8-hydroxy-2-oxo-1,2-dihydroquinolin-5-yl)ethylamino)ethyl)propanamide;
3-(2-(Biphenyl-3-yl)ethylamino)-N-cycloheptyl-N-(2-(2-(8-hydroxy-2-oxo-1,2-dihydroquinolin-5-yl)ethylamino)ethyl)propanamide;
3-(3-Chlorophenethylamino)-N-cyclohexyl-N-(2-(2-(8-hydroxy-2-oxo-1,2-dihydroquinolin-5-yl)ethylamino)ethyl)propanamide;
3-(4-Chlorophenethylamino)-N-cyclohexyl-N-(2-(2-(8-hydroxy-2-oxo-1,2-dihydroquinolin-5-yl)ethylamino)ethyl)propanamide;
N-Cyclohexyl-3-(3,5-difluorophenethylamino)-N-(2-(2-(8-hydroxy-2-oxo-1,2-dihydroquinolin-5-yl)ethylamino)ethyl)propanamide;
N-Cyclohexyl-3-(3,4-difluorophenethylamino)-N-(2-(2-(8-hydroxy-2-oxo-1,2-dihydroquinolin-5-yl)ethylamino)ethyl)propanamide;
N-Cyclohexyl-3-(2,5-difluorophenethylamino)-N-(2-(2-(8-hydroxy-2-oxo-1,2-dihydroquinolin-5-yl)ethylamino)ethyl)propanamide;
N-Cyclohexyl-3-(2,3-difluorophenethylamino)-N-(2-(2-(8-hydroxy-2-oxo-1,2-dihydroquinolin-5-yl)ethylamino)ethyl)propanamide;
N-Cyclohexyl-3-(3-fluorophenethylamino)-N-(2-(2-(8-hydroxy-2-oxo-1,2-dihydroquinolin-5-yl)ethylamino)ethyl)propanamide;
N-Cyclohexyl-3-(3-fluorophenethylamino)-N-(2-(2-(7-hydroxy-2-oxoindolin-4-yl)ethylamino)ethyl)propanamide;
N-Cyclohexyl-N-(2-(3-formamido-4-hydroxyphenethylamino)ethyl)-3-(phenethylamino)propanamide;
N-Cyclohexyl-N-(2-(3,4-dihydroxyphenethylamino)ethyl)-3-(phenethylamino)propanamide;
N-Cyclohexyl-N-(2-(2-(5-hydroxy-3-oxo-3,4-dihydro-2H-benzo[b][1,4]oxazin-8-yl)ethylamino)ethyl)-3-(phenethylamino)propanamide;
N-Cycloheptyl-N-(2-(2-(5-hydroxy-3-oxo-3,4-dihydro-2H-benzo[b][1,4]oxazin-8-yl)ethylamino)ethyl)-3-(phenethylamino)propanamide;
(R)—N-(Hexan-2-yl)-N-(2-(2-(5-hydroxy-3-oxo-3,4-dihydro-2H-benzo[b][1,4]oxazin-8-yl)ethylamino)ethyl)-3-(phenethylamino)propanamide;
N-Cycloheptyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide;
3-(3-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
3-(2-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(2,3-dichlorophenethylamino)-N-(2-(2-(5-hydroxy-3-oxo-3,4-dihydro-2H-benzo[b][1,4]oxazin-8-yl)ethylamino)ethyl)propanamide;
N-Cyclohexyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide;
N-Cyclohexyl-3-(2,4-dichlorophenethylamino)-N-(2-(2-(5-hydroxy-3-oxo-3,4-dihydro-2H-benzo[b][1,4]oxazin-8-yl)ethylamino)ethyl)propanamide;
3-(4-Chlorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(2-fluoro-5-methylphenethylamino)-N-(2-(2-(5-hydroxy-3-oxo-3,4-dihydro-2H-benzo[b][1,4]oxazin-8-yl)ethylamino)ethyl)propanamide;
3-(5-Chloro-2-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(3,5-difluorophenethylamino)-N-(2-(2-(5-hydroxy-3-oxo-3,4-dihydro-2H-benzo[b][1,4]oxazin-8-yl)ethylamino)ethyl)propanamide;
N-Cyclohexyl-3-(2,3-difluorophenethylamino)-N-(2-(2-(5-hydroxy-3-oxo-3,4-dihydro-2H-benzo[b][1,4]oxazin-8-yl)ethylamino)ethyl)propanamide;
3-(3-Chloro-4-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(2,5-difluorophenethylamino)-N-(2-(2-(5-hydroxy-3-oxo-3,4-dihydro-2H-benzo[b][1,4]oxazin-8-yl)ethylamino)ethyl)propanamide;
3-(3-Chloro-2-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(4-fluoro-3-methylphenethylamino)-N-(2-(2-(5-hydroxy-3-oxo-3,4-dihydro-2H-benzo[b][1,4]oxazin-8-yl)ethylamino)ethyl)propanamide;
3-(3-Chloro-5-fluorophenethylamino)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(3,4-difluorophenethylamino)-N-(2-(2-(5-hydroxy-3-oxo-3,4-dihydro-2H-benzo[b][1,4]oxazin-8-yl)ethylamino)ethyl)propanamide;
4-(2-(3-(Cyclohexyl(2-(2-(5-hydroxy-3-oxo-3,4-dihydro-2H-benzo[b][1,4]oxazin-8-yl)ethylamino)ethyl)amino)-3-oxopropylamino)ethyl)benzoic acid;
(R)-3-(3-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(4-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2-Chlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(4-Fluoro-3-methylphenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3-Chloro-4-fluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2-(5-Chlorothiophen-2-yl)ethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3-Chloro-5-fluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3,5-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3,4-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2,3-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2,5-Difluorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(3,4-Dichlorophenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(2-Fluoro-5-methylphenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)-3-(5-Fluoro-2-methylphenethylamino)-N-(hexan-2-yl)-N-(2-(2-(5-hydroxy-3-oxo-3,4-dihydro-2H-benzo[b][1,4]oxazin-8-yl)ethylamino)ethyl)propanamide;
(R)—N-sec-Butyl-3-(3,4-dichlorophenethylamino)-N-(2-(2-(5-hydroxy-3-oxo-3,4-dihydro-2H-benzo[b][1,4]oxazin-8-yl)ethylamino)ethyl)propanamide;
(R)-3-(3,4-Dichlorophenethylamino)-N-(2-(2-(5-hydroxy-3-oxo-3,4-dihydro-2H-benzo[b][1,4]oxazin-8-yl)ethylamino)ethyl)-N-(3-methylbutan-2-yl)propanamide;
(R)—N-(Heptan-2-yl)-N-(2-(2-(5-hydroxy-3-oxo-3,4-dihydro-2H-benzo[b][1,4]oxazin-8-yl)ethylamino)ethyl)-3-(phenethylamino)propanamide;
(R)—N-(1-Cyclohexylethyl)-N-(2-(2-(5-hydroxy-3-oxo-3,4-dihydro-2H-benzo[b][1,4]oxazin-8-yl)ethylamino)ethyl)-3-(phenethylamino)propanamide;
N-Cycloheptyl-3-(4-fluoro-3-methylphenethylamino)-N-(2-(2-(5-hydroxy-3-oxo-3,4-dihydro-2H-benzo[b][1,4]oxazin-8-yl)ethylamino)ethyl)propanamide;
3-(4-Chlorophenethylamino)-N-cycloheptyl-N-(2-(2-(5-hydroxy-3-oxo-3,4-dihydro-2H-benzo[b][1,4]oxazin-8-yl)ethylamino)ethyl)propanamide;
N-Cycloheptyl-3-(2,5-difluorophenethylamino)-N-(2-(2-(5-hydroxy-3-oxo-3,4-dihydro-2H-benzo[b][1,4]oxazin-8-yl)ethylamino)ethyl)propanamide;
N-Cycloheptyl-3-(3,5-difluorophenethylamino)-N-(2-(2-(5-hydroxy-3-oxo-3,4-dihydro-2H-benzo[b][1,4]oxazin-8-yl)ethylamino)ethyl)propanamide;
3-(3-Chloro-5-fluorophenethylamino)-N-cycloheptyl-N-(2-(2-(5-hydroxy-3-oxo-3,4-dihydro-2H-benzo[b][1,4]oxazin-8-yl)ethylamino)ethyl)propanamide;
N-Cycloheptyl-3-(3,4-difluorophenethylamino)-N-(2-(2-(5-hydroxy-3-oxo-3,4-dihydro-2H-benzo[b][1,4]oxazin-8-yl)ethylamino)ethyl)propanamide;
3-(3-Chlorophenethylamino)-N-cycloheptyl-N-(2-(2-(5-hydroxy-3-oxo-3,4-dihydro-2H-benzo[b][1,4]oxazin-8-yl)ethylamino)ethyl)propanamide;
N-Cycloheptyl-3-(3-fluorophenethylamino)-N-(2-(2-(5-hydroxy-3-oxo-3,4-dihydro-2H-benzo[b][1,4]oxazin-8-yl)ethylamino)ethyl)propanamide;
N-Cyclohexyl-N-(2-(2-(5-hydroxy-3-oxo-3,4-dihydro-2H-benzo[b][1,4]oxazin-8-yl)ethylamino)ethyl)-3-phenethoxypropanamide;
N-Cyclohexyl-3-(4-fluorophenethoxy)-N-(2-(2-(8-hydroxy-2-oxo-1,2-dihydroquinolin-5-yl)ethylamino)ethyl)propanamide;
N-Cyclohexyl-3-(3-fluorophenethoxy)-N-(2-(2-(8-hydroxy-2-oxo-1,2-dihydroquinolin-5-yl)ethylamino)ethyl)propanamide;
N-Cyclohexyl-3-(2-fluorophenethoxy)-N-(2-(2-(8-hydroxy-2-oxo-1,2-dihydroquinolin-5-yl)ethylamino)ethyl)propanamide;
N-Cyclohexyl-3-(3-fluorophenethoxy)-N-(2-(2-(5-hydroxy-3-oxo-3,4-dihydro-2H-benzo[b][1,4]oxazin-8-yl)ethylamino)ethyl)propanamide;
N-Cyclohexyl-3-(4-fluorophenethoxy)-N-(2-(2-(5-hydroxy-3-oxo-3,4-dihydro-2H-benzo[b][1,4]oxazin-8-yl)ethylamino)ethyl)propanamide;
N-Cyclohexyl-3-(2-fluorophenethoxy)-N-(2-(2-(5-hydroxy-3-oxo-3,4-dihydro-2H-benzo[b][1,4]oxazin-8-yl)ethylamino)ethyl)propanamide;
3-(3-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide;
N-Cyclohexyl-3-(3,5-difluorophenethoxy)-N-(2-(2-(8-hydroxy-2-oxo-1,2-dihydroquinolin-5-yl)ethylamino)ethyl)propanamide;
(R)—N-sec-Butyl-N-(2-(2-(8-hydroxy-2-oxo-1,2-dihydroquinolin-5-yl)ethylamino)ethyl)-3-phenethoxypropanamide;
(R)—N-(2-(2-(8-Hydroxy-2-oxo-1,2-dihydroquinolin-5-yl)ethylamino)ethyl)-N-(3-methylbutan-2-yl)-3-phenethoxypropanamide;
N-Cycloheptyl-N-(2-(2-(8-hydroxy-2-oxo-1,2-dihydroquinolin-5-yl)ethylamino)ethyl)-3-phenethoxypropanamide;
N-(2-(2-(8-Hydroxy-2-oxo-1,2-dihydroquinolin-5-yl)ethylamino)ethyl)-N-isopropyl-3-phenethoxypropanamide;
(R)—N-(Hexan-2-yl)-N-(2-(2-(8-hydroxy-2-oxo-1,2-dihydroquinolin-5-yl)ethylamino)ethyl)-3-phenethoxypropanamide;
N-(2-(2-(8-Hydroxy-2-oxo-1,2-dihydroquinolin-5-yl)ethylamino)ethyl)-N-neopentyl-3-phenethoxypropanamide;
N-Cyclohexyl-3-(2,4-difluorophenethoxy)-N-(2-(2-(8-hydroxy-2-oxo-1,2-dihydroquinolin-5-yl)ethylamino)ethyl)propanamide;
N-Cyclohexyl-3-(2,3-difluorophenethoxy)-N-(2-(2-(8-hydroxy-2-oxo-1,2-dihydroquinolin-5-yl)ethylamino)ethyl)propanamide;
N-Cyclohexyl-3-(3,4-difluorophenethoxy)-N-(2-(2-(8-hydroxy-2-oxo-1,2-dihydroquinolin-5-yl)ethylamino)ethyl)propanamide;
N-Cyclohexyl-3-(2,6-difluorophenethoxy)-N-(2-(2-(8-hydroxy-2-oxo-1,2-dihydroquinolin-5-yl)ethylamino)ethyl)propanamide;
N-Cyclohexyl-3-(2,5-difluorophenethoxy)-N-(2-(2-(8-hydroxy-2-oxo-1,2-dihydroquinolin-5-yl)ethylamino)ethyl)propanamide;
N-cyclohexyl-3-(2,4-difluorophenethoxy)-N-(2-(2-(5-hydroxy-3-oxo-3,4-dihydro-2H-benzo[b][1,4]oxazin-8-yl)ethylamino)ethyl)propanamide;
N-Cyclohexyl-3-(3,4-difluorophenethoxy)-N-(2-(2-(5-hydroxy-3-oxo-3,4-dihydro-2H-benzo[b][1,4]oxazin-8-yl)ethylamino)ethyl)propanamide;
N-Cyclohexyl-3-(2,3-difluorophenethoxy)-N-(2-(2-(5-hydroxy-3-oxo-3,4-dihydro-2H-benzo[b][1,4]oxazin-8-yl)ethylamino)ethyl)propanamide;
3-(2-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide;
3-(4-Chlorophenethoxy)-N-cyclohexyl-N-(2-(2-(5-hydroxy-3-oxo-3,4-dihydro-2H-benzo[b][1,4]oxazin-8-yl)ethylamino)ethyl)propanamide;
N-Cycloheptyl-N-(2-(2-(5-hydroxy-3-oxo-3,4-dihydro-2H-benzo[b][1,4]oxazin-8-yl)ethylamino)ethyl)-3-phenethoxypropanamide;
N-Cycloheptyl-N-(2-(2-(5-hydroxy-3-oxo-3,4-dihydro-2H-benzo[b][1,4]oxazin-8-yl)ethylamino)ethyl)-3-(2-(pyridin-2-yl)ethoxy)propanamide;
N-(2-(2-(5-Hydroxy-3-oxo-3,4-dihydro-2H-benzo[b][1,4]oxazin-8-yl)ethylamino)ethyl)-N-isopropyl-3-phenethoxypropanamide;
(R)—N-(2-(2-(5-Hydroxy-3-oxo-3,4-dihydro-2H-benzo[b][1,4]oxazin-8-yl)ethylamino)ethyl)-N-(3-methylbutan-2-yl)-3-phenethoxypropanamide;
N-Cyclopentyl-N-(2-(2-(5-hydroxy-3-oxo-3,4-dihydro-2H-benzo[b][1,4]oxazin-8-yl)ethylamino)ethyl)-3-phenethoxypropanamide;
N-(2-(2-(5-Hydroxy-3-oxo-3,4-dihydro-2H-benzo[b][1,4]oxazin-8-yl)ethylamino)ethyl)-N-neopentyl-3-phenethoxypropanamide;
3-(3-Chlorophenethoxy)-N-(2-(diethylamino)ethyl)-N-(2-(2-(5-hydroxy-3-oxo-3,4-dihydro-2H-benzo[b][1,4]oxazin-8-yl)ethylamino)ethyl)propanamide; or 3-(3-chlorophenethoxy)-N-(2-(diethylamino)ethyl)-N-(2-(2-(8-hydroxy-2-oxo-1,2-dihydroquinolin-5-yl)ethylamino)ethyl)propanamide.

13. A process for the preparation of a compound of formula (I) or a pharmaceutically acceptable salt thereof as claimed in claim 1, wherein:
the process comprises:
a) reacting a compound of formula (II) with a compound of formula (III) or a suitable salt thereof in the presence of a base,
b) when R² and R³ are both hydrogen, reacting a compound of formula (IV) with a compound of formula (III) or a suitable salt thereof in the presence of a suitable reducing agent,
c) when R² and R³ are both hydrogen, contacting a compound of formula (V) with a suitable reducing agent, or
d) when R², R³, R⁴, and R⁵ are all hydrogen, k is zero, m is 1, A is C(O), E is CH₂CH₂, and D is NH, reacting a compound, R⁷CH₂CH₂NH₂, with the following compound under Michael addition conditions: 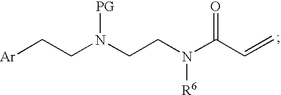
the compound of formula (II) is: 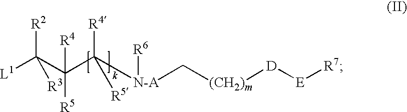
L¹ is leaving group;
the compound of formula (III) is: 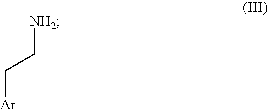
the compound of formula (IV) is: 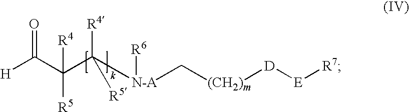
the compound of formula (V) is: 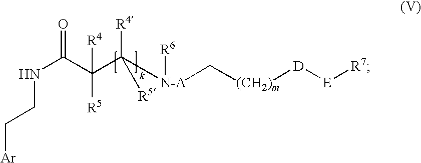
and
PG is a Protecting Group.

14. A process for the preparation of a salt of the compound of formula (XX), wherein:
the compound of formula (XX) is: 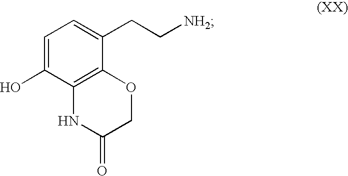
the process comprises reducing a compound of formula (XXI) with hydrogen in the presence of a suitable palladium catalyst in an aqueous medium and in the presence of a suitable strong acid; and
the compound of formula (XXI) is:

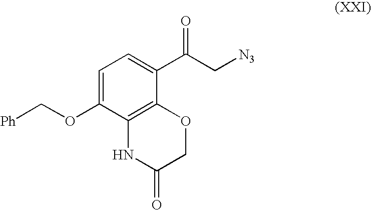

15. A compound, wherein:
the compound corresponds to formula (XXII): 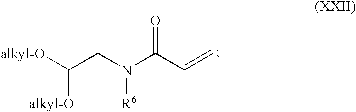
R⁶ is:
—(X)ₚ—Y—(Z), —R¹⁰, or
an α- or β-branched C₃₋₁₂ alkyl, wherein:
the α- or β-branched C₃₋₁₂ alkyl is optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁵⁸R⁵⁹, OC(O)(C₁₋₆ alkyl), C₃₋₁₂ cycloalkyl, or R⁶⁰; and
X and Z are, independently, a C₁₋₆ alkylene group, wherein:
the C₁₋₆ alkylene group is optionally substituted with halogen, trifluoromethyl, NH₂, (di)-C₁₋₆ alkylamino, (di)-C₁₋₆ alkylaminocarbonyl, C₁₋₆ alkylcarbonylamino, —SO₂NH₂, or (di)-C₁₋₆ alkylaminosulphonyl;
p and cl are, independently, zero or 1;
when p is 1, Y is a bond, oxygen, sulphur, CH₂, C(O), or NR⁹;
when p is zero, Y is a bond, oxygen, CH₂, C(O), or NR⁹;
R⁶⁰ is a 4- to 7-membered heterocyclyl, wherein:
the heterocyclyl comprises a ring nitrogen, oxygen, or sulphur, wherein:
the nitrogen is optionally substituted by C(O)(C₁₋₆ alkyl);
the heterocyclyl ring is optionally substituted by C₁₋₆ alkyl, and
ring carbon atoms of the heterocyclyl which are not adjacent to a ring heteroatom are optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁶¹R⁶², OC(O)(C₁₋₆ alkyl, or C₃₋₁₂ cycloalkyl;
R¹⁰ is hydrogen or a saturated or unsaturated 3- to 12-membered ring system, wherein:
the saturated or unsaturated 3- to 12-membered ring system optionally comprises a ring heteroatom selected from nitrogen, oxygen, and sulphur, and
the saturated or unsaturated 3- to 12-membered ring system is optionally substituted by halogen, trifluoromethyl, cyano, carboxyl, hydroxy, —S(O)ᵣR¹⁵, —NR¹⁶S(O)ₛR¹⁷, —C(O)NR¹⁸R¹⁹, —NHC(O)R²⁰, C₁₋₆ alkyl, C₁₋₆ alkoxy, C₁₋₆ alkylcarbonyl, C₁₋₆ alkoxycarbonyl, or a saturated or unsaturated 4- to 7-membered monocyclic ring system, wherein:
the saturated or unsaturated 4- to 7-membered monocyclic ring system optionally comprises at least one ring heteroatom selected from nitrogen, oxygen, and sulphur, and
the saturated or unsaturated 4- to 7-membered monocyclic ring system is optionally substituted by halogen, trifluoromethyl, hydroxy, —NR²¹S(O)ₜR²², —NHC(O)R²³, or C₁₋₆ alkoxy;
R⁹, R¹⁶, R¹⁸, R¹⁹, R²⁰, R²¹, R²³, R⁵⁸, R⁵⁹, R⁶⁰, and R⁶² are, independently hydrogen or C₁₋₆ alkyl;
R¹⁵, R¹⁷, and R²² are, independently, C₁₋₆ alkyl;
r is zero, 1, or 2;
s and t are 2; and
the “alkyl” substituents depicted in formula (XXII) are C₁₋₁₀ alkyl.

16. A compound, wherein:
the compound corresponds to formula (XXIII): 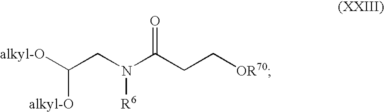
R⁶ is:
—(X)ₚ—Y—(Z)_{q}—R¹⁰, or
an α- or β-branched C₃₋₁₂ alkyl, wherein:
the α- or β-branched C₃₋₁₂ alkyl is optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁵⁸R⁵⁹, OC(O)(C₁₋₆ alkyl), C₃₋₁₂ cycloalkyl, or R⁶⁰; and
X and Z are, independently, a C₁₋₆ alkylene group, wherein:
the C₁₋₆ alkylene group is optionally substituted with halogen, trifluoromethyl, NH₂, (di)-C₁₋₆ alkylamino, (di)-C₁₋₆ alkylaminocarbonyl, C₁₋₆ alkylcarbonylamino, —SO₂NH₂, or (di)-C₁₋₆ alkylaminosulphonyl;
p and cl are, independently, zero or 1;
when p is 1, Y is a bond, oxygen, sulphur, CH₂, C(O), or NR⁹;
when p is zero, Y is a bond, oxygen, CH₂, C(O), or NR⁹;
R⁶⁰ is a 4- to 7-membered heterocyclyl, wherein:
the heterocyclyl comprises a ring nitrogen, oxygen, or sulphur, wherein:
the nitrogen is optionally substituted by C(O)(C₁₋₆ alkyl);
the heterocyclyl ring is optionally substituted by C₁₋₆ alkyl, and
ring carbon atoms of the heterocyclyl which are not adjacent to a ring heteroatom are optionally substituted by halogen, C₁₋₆ alkoxy, C₁₋₆ alkylthio, C₁₋₆ alkylS(O), C₁₋₆ alkylS(O)₂, C₁₋₆ haloalkoxy, hydroxy, NR⁶¹R⁶², OC(O)(C₁₋₆ alkyl, or C₃₋₁₂ cycloalkyl;
R¹⁰ is hydrogen or a saturated or unsaturated 3- to 12-membered ring system, wherein:
the saturated or unsaturated 3- to 12-membered ring system optionally comprises a ring heteroatom selected from nitrogen, oxygen, and sulphur, and
the saturated or unsaturated 3- to 12-membered ring system is optionally substituted by halogen, trifluoromethyl, cyano, carboxyl, hydroxy, —S(O)ᵣR¹⁵, —NR¹⁶S(O)ₛR¹⁷, —C(O)NR¹⁸R¹⁹, —NHC(O)R²⁰, C₁₋₆ alkyl, C₁₋₆ alkoxy, C₁₋₆ alkylcarbonyl, C₁₋₆ alkoxycarbonyl, or a saturated or unsaturated 4- to 7-membered monocyclic ring system, wherein:
the saturated or unsaturated 4- to 7-membered monocyclic ring system optionally comprises at least one ring heteroatom selected from nitrogen, oxygen, and sulphur, and
the saturated or unsaturated 4- to 7-membered monocyclic ring system is optionally substituted by halogen, trifluoromethyl, hydroxy, —NR²¹S(O)ₜR²², —NHC(O)R²³, or C₁₋₆ alkoxy;
R⁹, R¹⁶, R¹⁸, R¹⁹, R²⁰, R²¹, R²³, R⁵⁸, R⁵⁹, R⁶¹, and R⁶² are, independently, hydrogen or C₁₋₆ alkyl;
R¹⁵, R¹⁷, and R²² are, independently, C₁₋₆ alkyl;
r is zero, 1, or 2;
s and t are 2;
R⁷⁰ is C₁₋₆ alkyl; and
the “alkyl” substituents depicted in formula (XXIII) are C₁₋₁₀ alkyl.

17. A pharmaceutical composition, wherein the pharmaceutical composition comprises:
a compound of formula (I) or a pharmaceutically acceptable salt thereof as claimed in claim 1;
a pharmaceutically acceptable adjuvant, diluent, or carrier.

18. A process for the preparation of a pharmaceutical composition, wherein the process comprises mixing a compound of formula (I) or a pharmaceutically acceptable salt thereof as claimed in claim 1 with a pharmaceutically acceptable adjuvant, diluent, or carrier.

19-21. (canceled)

22. A method of treating, or reducing the risk of, a disease or condition in which modulation of β2 adrenoreceptor activity is beneficial, wherein the method comprises administering to a patient in need thereof a therapeutically effective amount of a compound of formula (I) or a pharmaceutically acceptable salt thereof as claimed in claim 1.

23. A method of treating, or reducing the risk of, an inflammatory disease or condition, wherein the method comprises administering to a patient in need thereof a therapeutically effective amount of a compound of formula (I) or a pharmaceutically acceptable salt thereof as claimed in claim 1.

24. A method according to claim 22, wherein the disease or condition is adult respiratory distress syndrome (ARDS), pulmonary emphysema, bronchitis, bronchiectasis, chronic obstructive pulmonary disease (COPD), asthma, or rhinitis.

25. A combination, wherein the combination comprises:
a compound of formula (I) or a pharmaceutically acceptable salt thereof as claimed in claim 1; and
one or more agents selected from:
a non-steroidal glucocorticoid receptor (GR-receptor) agonist;
a steroid;
a PDE4 inhibitor;
a muscarinic receptor antagonist;
a modulator of chemokine receptor function; and
an inhibitor of a kinase function.
